(19) United States
(12) Patent Application Publication
(10) Pub. No.: US 2026/0207560 A1
(43) Pub. Date: Jul. 23, 2026

(54) PLURALITY OF GRANULES

(71) Applicant: ARTELO BIOSCIENCES LIMITED, Alderley Edge, Cheshire (GB)

(72) Inventors: Andrew YATES, Alderley Edge (GB); Robert WARD, Deeside (GB); Paul DICKINSON, Cheadle (GB)

(73) Assignee: ARTELO BIOSCIENCES LIMITED, Alderley Edge, Cheshire (GB)

(21) Appl. No.: 18/872,607

(22) PCT Filed: Jun. 7, 2023
(86) PCT No.: PCT/GB2023/051480

§ 371 Date: Dec. 6, 2024

(30) Foreign Application Priority Data
Jun. 7, 2022 (GB) 2208352.1

(51) Int. Cl. A61K 31/4184; A61K 9/16; A61K 9/48

(52) U.S. Cl. CPC A61K 31/4184; A61K 9/1623; A61K 9/1652; A61K 9/48

(57) ABSTRACT

The present disclosure relates to a plurality of granules comprising N-(2-(tert-butyl)-1-((4,4-difluorocyclohexyl)methyl)-1H-benzimidazol-5-yl)ethanesulfonamide, or a pharmaceutically acceptable salt thereof, as an active substance. The plurality of granules are suitable for use in pharmaceutical compositions. The pharmaceutical compositions are suitable for use in the treatment of medical conditions in which treatment with agonists of CB₁/CB₂ receptors are beneficial.

### FIELD OF THE DISCLOSURE

The present disclosure relates to a plurality of granules comprising N-(2-(tert-butyl)-1-((4,4-difluorocyclohexyl)methyl)-1H-benzimidazol-5-yl)ethanesulfonamide, or a pharmaceutically acceptable salt thereof, as an active substance. The plurality of granules are suitable for use in pharmaceutical compositions. The pharmaceutical compositions are suitable for use in the treatment of medical conditions in which treatment with agonists of CB₁/CB₂ receptors are beneficial.

### BACKGROUND OF THE DISCLOSURE

N-(2-(tert-butyl)-1-((4,4-difluorocyclohexyl)methyl)-1H-benzimidazol-5 yl)ethanesulfonamide is a highly potent, peripherally restricted synthetic, dual cannabinoid agonist which targets the peripheral CB₁/CB₂ receptors (Groblewski T, Yu X H, Lessard E. Pre-clinical pharmacological properties of novel peripherally acting CB1-CB2 agonists. 20th Annual Symposium of the International Cannabinoid Research Society; 2010; Abstract #37). The structure of N-(2-(tert-butyl)-1-((4,4-difluorocyclohexyl)methyl)-1H-benzimidazol-5-yl)ethanesulfonamide (Compound I) is shown below.

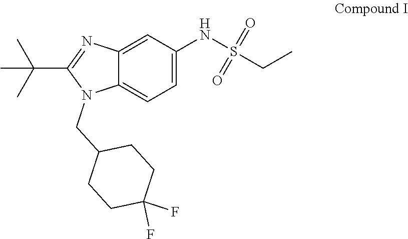 [SMILES: CCS(=O)(=O)Nc1ccc2c(c1)nc(C(C)(C)C)n2CC1CCC(F)(F)CC1]

Compound I and a synthetic route to prepare it were first disclosed in WO2006/033631 A1. Compound I has been evaluated in a number of phase 1 clinical trials as a potential treatment for pain.

Cannabinoid agonists have potential uses in a number of clinical settings, which include pain relief and cancer therapy (Cancer Prev Res (Phila). 2011 January; 4(1): 65-75).

Furthermore, multiple cannabinoid agonists have been approved in the US and other major markets for the treatment of nausea and vomiting related to cancer, and clinical studies have been conducted which evaluate the potential for cannabinoids to be used for anorexia and cachexia associated with cancer.

Compound I has been shown to stimulate appetite in a dose-dependent manner. Compound I acts peripherally on the part of the endocannabinoid system that stimulates hunger and does not cause CNS-psychoactive effects.

Due to the high potency of Compound I, very low therapeutic doses of the active substance (in the microgram range) are required. Pharmaceutical compositions requiring very low amounts of an active substance are difficult to manufacture as it can be difficult to achieve an acceptable level of content uniformity. It is imperative that pharmaceutical compositions have an acceptable content uniformity as it ensures that the patient receiving the pharmaceutical composition receives the intended dose. This is critical from a safety and an efficacy perspective. In the present case it is anticipated the lowest dose could be as low as 50 μg.

Because of the very low amounts of active substance needed, problems can arise during mixing and formulation of the active substance, for example due to segregation, content uniformity and physical stability. The small quantity of the active substance required must be evenly distributed throughout a powder blend. This is especially difficult if the active substance is poor flowing and/or cohesive.

In the present case, an immediate release pharmaceutical composition comprising Compound I is also required to provide optimal absorption.

Furthermore, Compound I is prone to degradation when present in a solution and on exposure to light, see Example 1 herein. This is problematic when considering viable pharmaceutical compositions for this active substance. The active substance needs to be stable during the manufacturing process of the pharmaceutical composition and preferably the resultant composition must also be stable over an extended period of time.

The combination of requiring a low dose of Compound I and its instability in solution present difficulties when it comes to developing a suitable pharmaceutical composition.

Thus, there is a need for an immediate release oral dosage form of Compound I with good uniformity of content, and good physical and chemical stability during manufacture of the pharmaceutical composition and preferably over prolonged period of times.

The present disclosure was devised with the foregoing in mind.

### SUMMARY OF THE DISCLOSURE

Disclosed herein are plurality of granules comprising as an active substance Compound I, or a pharmaceutically acceptable salt thereof, and pharmaceutical compositions comprising the plurality of granules.

According to a first aspect of the disclosure, there is provided a plurality of granules comprising
a. N-(2-(tert-butyl)-1-((4,4-difluorocyclohexyl)methyl)-1H-benzimidazol-5-yl)ethanesulfonamide, or a pharmaceutically acceptable salt thereof; and
b. at least one pharmaceutically acceptable excipient.

According to a second aspect of the disclosure, there is provided a pharmaceutical composition comprising the plurality of granules as herein defined.

In a further aspect, there is a process to prepare the plurality of granules as herein defined, the process comprising:
a. mixing a binder and diluent together;
b. adding a primary granulation fluid to the mixture from step a. and mixing until granules are formed; and
c. drying the mixture from step b;
   wherein N-(2-(tert-butyl)-1-((4,4-difluorocyclohexyl)methyl)-1H-benzimidazol-5-yl)ethanesulfonamide, or a pharmaceutically acceptable salt thereof, is either mixed with the binder and diluent in step a. or dissolved in the primary granulation fluid.

In a further aspect, there is provided a pharmaceutical composition as defined herein for use as a medicament.

Preferred, suitable, and optional features of any one particular aspect of the present disclosure are also preferred, suitable, and optional features of any other aspect.

### BRIEF DESCRIPTION OF THE DRAWINGS

For a better understanding of the disclosure, and to show how embodiments of the same are put into effect, reference is now made, by way of example, to the following figures, in which:

FIG. 1: Drug release from 50 μg HPMC capsules (Quali-V) (TRI002/89G50-4A Vs TRI002/89G50-4B) in 0.1M HCl medium

FIG. 2: Drug release from 50 μg HPMC capsules (Quali-V) (TRI002/89G50-4A Vs TRI002/89G50-4B) in pH 6.8 phosphate buffer medium

FIG. 3: Drug release from n=6 50 μg capsules (TRI002/89G50-4H) in 0.1M HCl medium

FIG. 4: Drug release from n=3 50 μg capsules (TRI002/89G50-4H) in pH 6.8 medium

FIG. 5: Mean Drug release from 50 μg capsules (TRI002/89G50-4H) in 0.1M HCl vs pH 6.8 media

FIG. 6: Drug release from n=6 200 μg capsules (TRI002/89G200-2H) in 0.1M HCl medium

FIG. 7: Drug release from n=3 200 μg capsules (TRI002/89G200-2H) in pH 6.8 medium

FIG. 8: Mean Drug release from 200 μg capsules (TRI002/89G200-2H) in HCl vs pH 6.8 media

### DETAILED DESCRIPTION OF THE DISCLOSURE

Unless otherwise stated, the following terms used in the specification and claims have the following meanings set out below.

The disclosed plurality of granules, compositions, processes of manufacture and methods may be understood more readily by reference to the following detailed description taken in connection with the accompanying figures, which form a part of this disclosure. It is to be understood that the disclosed plurality of granules, compositions, processes of manufacture and methods are not limited to the specific plurality of granules, compositions, processes of manufacture and methods described and/or shown herein, and that the terminology used herein is for the purpose of describing particular embodiments by way of example only and is not intended to be limiting of the claimed plurality of granules, compositions, processes of manufacture and methods

Reference to a particular numerical value includes at least that particular value, unless the context clearly dictates otherwise. When a range of values is expressed, another embodiment includes from the one particular value and/or to the other particular value. Further, reference to values stated in ranges include each and every value within that range. All ranges are inclusive and combinable.

When values are expressed as approximations, by use of the antecedent “about,” it will be understood that the particular value forms another embodiment.

The term “about” when used in reference to numerical ranges, cut-offs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value.

It is to be appreciated that certain features of the disclosed plurality of granules, compositions, processes of manufacture and methods which are, for clarity, described herein in the context of separate embodiments, may also be provided in combination in a single embodiment. Conversely, various features of the disclosed plurality of granules, compositions, processes of manufacture and methods that are, for brevity, described in the context of a single embodiment, may also be provided separately or in any sub-combination.

As used herein, the singular forms “a,” “an,” and “the” include the plural.

The active substance is N-(2-(tert-butyl)-1-((4,4-difluorocyclohexyl)methyl)-1H-benzimidazol-5-yl)ethanesulfonamide (Compound I) or a pharmaceutically acceptable salt thereof.

Throughout the specification, unless specified otherwise, references to the amount of the active substance will be understood to refer to the amount of the parent compound on a free base basis, even if the compound is present as a salt of Compound 1.

Where an item is said to comprise a stipulated ingredient, it is to include the item comprising one type of the stipulated ingredient, or a mixture of types of the stipulated ingredients. For example, said item may comprise at least one type of the stipulated ingredient, such as at least two types, at least three types, or at least four types. Suitably, the item comprises one type of the stipulated ingredient, two types of the stipulated ingredients, three types of the stipulated ingredients or four types of the stipulated ingredients.

Where an item is said to comprise a stipulated ingredient (optionally in a stipulated amount or concentration), said item may optionally include additional ingredients other than that stipulated. However, in certain embodiments, the item said to comprise a stipulated ingredient may in fact consist essentially of or consist of all the stipulated ingredient.

Herein, when an item is said to “consists essentially of” a particular component, said item suitably comprises at least 70 wt % of said component, suitably at least 80 wt % thereof, suitably at least 90 wt % thereof, suitably at least 95 wt % thereof, most suitably at least 99 wt % thereof. Suitably, when an item is said to “consist essentially of” a particular component consists of said component save for one or more trace impurities.

Room temperature is herein defined as a temperature of about 15° C. to about 25° C., such as about 20° C. to about 25° C. Suitably, room temperature is about 20° C. Suitably, room temperature is 15° C. to 25° C., such as 20° C. to 25° C. Suitably, room temperature is 20° C.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

As used herein, the phrase “therapeutically effective dose” refers to an amount of the pharmaceutical composition comprising the active substance, as described herein, effective to achieve a particular biological or therapeutic result such as, but not limited to, biological or therapeutic results disclosed, described, or exemplified herein. The therapeutically effective dose may vary according to factors such as the disease state, age, sex, and weight of the individual, and the ability of the composition to cause a desired response in a subject. Such results include, but are not limited to, the reduction, remission, and/or regression of the benign or malignant disease or prevention of the development of the benign or malignant disease, as determined by any means suitable in the art.

As used herein, “subject” includes a vertebrate; mammal, such as primates, humans, dogs, cattle, and horses; or a domestic animal.

### Plurality of Granules

According to a first aspect of the disclosure, there is provided a plurality of granules comprising:
a. N-(2-(tert-butyl)-1-((4,4-difluorocyclohexyl)methyl)-1H-benzimidazol-5-yl)ethanesulfonamide, or a pharmaceutically acceptable salt thereof; and
b. at least one pharmaceutically acceptable excipient.

Advantageously, the plurality of granules are formed using a wet granulation process—see Examples. The wet granulation process facilitates the active substance being uniformly distributed throughout the plurality of granules which is critical from a patient safety perspective as it ensures the intended amount of the active substance is present in a given portion of the plurality of granules, for example, in a pharmaceutical composition comprising a portion of the plurality of granules.

Suitably, the pharmaceutically acceptable excipient is selected from a diluent, disintegrant and binder.

Suitably, the plurality of granules comprise a diluent. Suitably, the plurality of granules comprises a binder. Suitably, the plurality of granules comprises a disintegrant.

Suitably, the plurality of granules comprise a diluent and binder.

Suitably, the plurality of granules comprise a diluent, binder and disintegrant.

Suitably, N-(2-(tert-butyl)-1-((4,4-difluorocyclohexyl)methyl)-1H-benzimidazol-5-yl)ethanesulfonamide, or a pharmaceutically acceptable salt thereof, is present in the plurality granules in an amount of less than 0.5% w/w by weight of the total weight of the plurality of granules, such as less than 0.4, 0.3, 0.2, 0.15, 0.14, 0.13, 0.12, 0.11, 0.10, 0.08, 0.06, or 0.05% w/w.

Suitably, N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of between about 0.01% w/w and about 0.5% w/w by weight of the total weight of the plurality of granules, such as between about 0.01% w/w and about 0.4% w/w, about 0.01% w/w and about 0.3% w/w, about 0.01% w/w and about 0.2% w/w, about 0.01% w/w and about 0.15% w/w, about 0.01% w/w and about 0.12% w/w, about 0.02% w/w and about 0.12% w/w, or about 0.03% w/w and about 0.10% w/w.

Suitably, N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of about 0.04, about 0.10 or about 0.12% w/w of the total weight of the plurality of granules.

Suitably, the diluent is present in the plurality of granules in an amount of at least 70% w/w by weight of the total weight of the plurality of granules, such as at least 75, 80, 85, or 90% w/w.

Suitably, the diluent is present in the plurality of granules in an amount of between about 75% w/w and about 98% w/w by weight of the total weight of the plurality of granules, such as between about 85% w/w and about 98% w/w or between about 90% w/w and about 95% w/w.

Suitably, the diluent is present in the plurality of granules in an amount of about 93% by weight of the total weight of the plurality of granules.

Suitably, the binder is present in the plurality of granules in an amount of at least 0.5% w/w by weight of the total weight of the plurality of granules, such as at least 1 or 2% w/w.

Suitably, the binder is present in the plurality of granules in an amount between about 0.5% w/w and about 5% w/w by weight of the total weight of the plurality of granules, such as between about 1% w/w and about 4% w/w or between about 2% w/w and about 3% w/w.

Suitably, the binder is present in the plurality of granules in an amount of about 2.5% w/w by weight of the total weight of the plurality of granules.

Suitably, the disintegrant is present in the plurality of granules in an amount of at least 0.5% w/w by weight of the total weight of the plurality of granules, such as at least 1, 2, or 3% w/w.

Suitably, the disintegrant is present in the plurality of granules in an amount of between about 1% w/w and about 10% w/w by weight of the total weight of the plurality of granules, such as between about 2% w/w and about 6% w/w or between about 3% w/w and about 5% w/w.

Suitably, the disintegrant is present in the plurality of granules in an amount of about 4% w/w of the total weight of the plurality of granules.

Suitably, the plurality of granules comprise:
a. less than 0.5% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 70% w/w of a diluent; and
c. at least 0.5% w/w of a binder;
   wherein weights are relative to the total weight of the plurality of granules.

Suitably, the plurality of granules comprise:
a. less than 0.5% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 70% w/w of a diluent;
c. at least 0.5% w/w of a binder; and
d. at least 0.5% w/w of a disintegrant;
   wherein weights are relative to the total weight of the plurality of granules.

Suitably, the plurality of granules comprise:
a. less than 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 70% w/w of a diluent;
c. at least 0.5% w/w of a binder; and
d. at least 1% w/w of a disintegrant;
   wherein weights are relative to the total weight of the plurality of granules.

Suitably, the plurality of granules comprise:
a. less than 0.13% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 80% w/w of a diluent;
c. at least 1% w/w of a binder; and
d. at least 1% w/w of a disintegrant;
   wherein weights are relative to the total weight of the plurality of granules.

Suitably, the plurality of granules comprise:
a. less than 0.11% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 90% w/w of a diluent;
c. at least 1% w/w of a binder; and
d. at least 1% w/w of a disintegrant;
   wherein weights are relative to the total weight of the plurality of granules.

Suitably, the plurality of granules comprise:
a. less than 0.05% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 90% w/w of a diluent;
c. at least 1% w/w of a binder; and
d. at least 1% w/w of a disintegrant;
   wherein weights are relative to the total weight of the plurality of granules

Suitably, the plurality of granules comprise:
a. between about 0.01% w/w and about 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. between about 85% w/w and about 98% w/w of a diluent;
c. between about 2% w/w and about 3% of a binder; and
d. between about 2% w/w and about 6% w/w of a disintegrant;
   wherein weights are relative to the total weight of the plurality of granules.

Suitably, the plurality of granules comprise:
a. about 0.04% w/w of N-(2-(tert-butyl)-1-((4,4-difluorocyclohexyl)methyl)-1H-benzimidazol-5-yl)ethanesulfonamide, or a pharmaceutically acceptable salt thereof;
b. about 93% w/w of a diluent;
c. about 2.5% w/w of a binder; and
d. about 4% w/w of a disintegrant;
   wherein weights are relative to the total weight of the plurality of granules.

Suitably, the plurality of granules comprise:
a. about 0.1% w/w of N-(2-(tert-butyl)-1-((4,4-difluorocyclohexyl)methyl)-1H-benzimidazol-5-yl)ethanesulfonamide, or a pharmaceutically acceptable salt thereof;
b. about 93% w/w of a diluent;
c. about 2.5% w/w of a binder; and
d. about 4% w/w of a disintegrant;
   wherein weights are relative to the total weight of the plurality of granules.

Suitably, the plurality of granules comprise:
a. about 0.12% w/w of N-(2-(tert-butyl)-1-((4,4-difluorocyclohexyl)methyl)-1H-benzimidazol-5-yl)ethanesulfonamide, or a pharmaceutically acceptable salt thereof;
b. about 93% w/w of a diluent;
c. about 2.5% w/w of a binder; and
d. about 4% w/w of a disintegrant;
   wherein weights are relative to the total weight of the plurality of granules.

Suitably, the plurality of granules comprise:
a. N-(2-(tert-butyl)-1-((4,4-difluorocyclohexyl)methyl)-1H-benzimidazol-5-yl)ethanesulfonamide, or a pharmaceutically acceptable salt thereof;
b. mannitol;
c. hydroxypropyl methyl cellulose; and
d. low-substituted hydroxy propyl cellulose.

Suitably, the plurality of granules comprise:
a. between about 0.01% w/w and about 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. between about 85% w/w and about 98% w/w of mannitol;
c. between about 2% w/w and about 3% of hydroxypropyl methyl cellulose; and
d. between about 2% w/w and about 6% w/w of low-substituted hydroxy propyl cellulose;
   wherein weights are relative to the total weight of the plurality of granules.

Suitably, the plurality of granules further comprise a lubricant.

Suitably, the lubricant is present in the plurality of granules in an amount of at least 0.1% w/w by weight of the total weight of the plurality of granules, such as at least 0.5 or 0.8% w/w.

Suitably, the lubricant is present in the plurality of granules in an amount of between about 0.1% w/w and about 2% w/w by weight of the total weight of the plurality of granules, such as between about 0.5% w/w and about 1.5% w/w.

Suitably, the lubricant is present in the plurality of granules in an amount of about 1% w/w by weight of the total weight of the plurality of granules.

Suitably, the plurality of granules comprise:
a. less than 0.5% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 70% w/w of a diluent;
c. at least 0.5% w/w of a binder;
d. at least 0.5% w/w of a disintegrant; and
e. at least 0.1% w/w of a lubricant;
   wherein weights are relative to the total weight of the plurality of granules.

Suitably, the plurality of granules comprise:
a. less than 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 70% w/w of a diluent;
c. at least 0.5% w/w of a binder;
d. at least 1% w/w of a disintegrant; and
e. at least 0.5% w/w of a lubricant;
   wherein weights are relative to the total weight of the plurality of granules.

Suitably, the plurality of granules comprise:
a. between about 0.01% w/w and about 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. between about 85% w/w and about 98% w/w of a diluent;
c. between about 2% w/w and about 3% of a binder;
d. between about 2% w/w and about 6% w/w of a disintegrant; and
e. between about 0.5% w/w and about 1.5% w/w of a lubricant;
   wherein weights are relative to the total weight of the plurality of granules.

Suitably, the plurality of granules comprise:
a. about 0.04% w/w of N-(2-(tert-butyl)-1-((4,4-difluorocyclohexyl)methyl)-1H-benzimidazol-5-yl)ethanesulfonamide, or a pharmaceutically acceptable salt thereof;
b. about 93% w/w of a diluent;
c. about 2.5% w/w of a binder;
d. about 4% w/w of a disintegrant; and
e. about 1% w/w of a lubricant;
   wherein weights are relative to the total weight of the plurality of granules.

Suitably, the plurality of granules comprise:
a. about 0.1% w/w of N-(2-(tert-butyl)-1-((4,4-difluorocyclohexyl)methyl)-1H-benzimidazol-5-yl)ethanesulfonamide, or a pharmaceutically acceptable salt thereof;
b. about 93% w/w of a diluent;
c. about 2.5% w/w of a binder;
d. about 4% w/w of a disintegrant; and
e. about 1% w/w of a lubricant;
   wherein weights are relative to the total weight of the plurality of granules.

Suitably, the plurality of granules comprise:
a. N-(2-(tert-butyl)-1-((4,4-difluorocyclohexyl)methyl)-1H-benzimidazol-5-yl)ethanesulfonamide, or a pharmaceutically acceptable salt thereof;
b. mannitol;
c. hydroxypropyl methyl cellulose;
d. low-substituted hydroxy propyl cellulose; and
e. sodium stearyl fumarate.

Suitably, the plurality of granules comprise:
a. between about 0.01% w/w and about 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. between about 85% w/w and about 98% w/w of mannitol;
c. between about 2% w/w and about 3% of hydroxypropyl methyl cellulose;
d. between about 2% w/w and about 6% w/w of low-substituted hydroxy propyl cellulose; and
f. between about 0.5% w/w and about 1.5% w/w of sodium stearyl fumarate;
   wherein weights are relative to the total weight of the plurality of granules.

Suitably, the plurality of granules have a mean size of less than 1200 μm, suitably less than 1000 μm.

Suitably, the plurality of granules have a mean size of between 800 and 1200 μm, suitably between 1000 and 1200 μm.

Suitably, the plurality of granules as defined herein have a bulk density of between about 0.1 and about 0.7 g/mL, such as between about 0.2 and about 0.6 g/mL, or between about 0.3 and about 0.5 g/mL. Suitably, the plurality of granules as defined herein have a bulk density of about 0.4 g/mL

### Pharmaceutical Compositions

According to a second aspect of the disclosure, there is provided a pharmaceutical composition for oral use comprising the plurality of granules as herein disclosed.

Preferably, the pharmaceutical composition is an immediate release pharmaceutical composition.

Suitably, the pharmaceutical composition consists essentially of the plurality of granules as herein disclosed. Suitably, the pharmaceutical composition consists of the plurality of granules as herein disclosed.

Suitably, the pharmaceutical composition is a capsule or a tablet.

Preferably, the pharmaceutical composition is a capsule. Suitably, the pharmaceutical composition comprises a capsule shell and the plurality of granules as herein defined.

Suitably, the capsule shell comprises hydroxypropyl methyl cellulose or gelatin. Suitably, the capsule shell comprises gelatin, such as hard gelatin. Suitably, the capsule shell is a size 4 size capsule shell.

Suitably, the pharmaceutical composition is a tablet. Suitably, the tablet is made by compressing the plurality of granules as defined herein.

Suitably, the pharmaceutical composition comprises the plurality of granules as defined herein wherein the plurality of granules comprise between about 0.001 and about 0.8% w/w of the active substance by weight of the total weight of the plurality of granules, such as between about 0.01 and about 0.8% w/w, about 0.02 and about 0.2% w/w, or about 0.03 and about 0.15% w/w. Suitably, the pharmaceutical composition comprises the plurality of granules as defined herein wherein the plurality of granules comprises between 0.001 and 0.8% w/w of the active substance by weight of the total weight of the plurality of granules, such as between 0.01 and 0.8% w/w, 0.02 and 0.2% w/w, or 0.03 and 0.15% w/w.

Suitably, the pharmaceutical composition comprises the plurality of granules as defined herein wherein the plurality of granules comprises about 0.1% w/w of the active substance by weight of the total weight of the plurality of granules. Suitably, the pharmaceutical composition comprises the plurality of granules as defined herein wherein the plurality of granules comprises about 0.4% w/w of the active substance by weight of the total weight of the plurality of granules. Suitably, the pharmaceutical composition comprises the plurality of granules as defined herein wherein the plurality of granules comprises 0.1% w/w of the active substance by weight of the total weight of the plurality of granules. Suitably, the pharmaceutical composition comprises the plurality of granules as defined herein wherein the plurality of granules comprises 0.4% w/w of the active substance by weight of the total weight of the plurality of granules.

Suitably, there is provided a pharmaceutical composition for oral use comprising the plurality of granules as defined herein wherein the pharmaceutical composition comprises between about 10 μg and about 1000 μg of the active substance, such as between about 10 μg and about 500 μg, about 20 μg and about 500 μg, about 30 μg and about 300 μg, or about 40 μg and about 250 μg.

Suitably, there is provided a pharmaceutical composition for oral use comprising the plurality of granules as defined herein wherein the pharmaceutical composition comprises about 50 μg or about 200 μg of the active substance.

Suitably, the pharmaceutical composition comprises the plurality of granules as defined herein wherein the plurality of granules comprise a diluent. Suitably, the diluent is present in the in an amount of at least 70% w/w by weight of the total weight of the plurality of granules, such as at least 75, 80, 85, or 90% w/w.

Suitably, the diluent is present in the plurality of granules in an amount of between about 75% w/w and about 98% w/w by weight of the total weight of the plurality of granules, such as between about 85% w/w and about 98% w/w or between about 90% w/w and about 95% w/w.

Suitably, the diluent is present in the plurality of granules in an amount of about 93% by weight of the total weight of the plurality of granules.

Suitably, the pharmaceutical composition comprises the plurality of granules as defined herein wherein the plurality of granules comprises a binder. Suitably, the binder is present in the plurality of granules in an amount of at least 0.5% w/w by weight of the total weight of the plurality of granules, such as at least 1 or 2% w/w.

Suitably, the binder is present in the plurality of granules in an amount between about 0.5% w/w and about 5% w/w by weight of the total weight of the plurality of granules, such as between about 1% w/w and about 4% w/w or between about 2% w/w and about 3% w/w.

Suitably, the binder is present in the plurality of granules in an amount of about 2.5% w/w by weight of the total weight of the plurality of granules.

Suitably, the pharmaceutical composition comprises the plurality of granules as defined herein wherein the plurality of granules comprises a disintegrant. Suitably, the disintegrant is present in the plurality of granules in an amount of at least 0.5% w/w by weight of the total weight of the plurality of granules, such as at least 1, 2, or 3% w/w.

Suitably, the disintegrant is present in the plurality of granules in an amount of between about 1% w/w and about 10% w/w by weight of the total weight of the plurality of granules, such as between about 2% w/w and about 6% w/w or between about 3% w/w and about 5% w/w.

Suitably, the disintegrant is present in the plurality of granules in an amount of about 4% w/w by weight of the total weight of the plurality of granules.

Suitably, there is provided a pharmaceutical composition for oral use comprising the plurality of granules as defined herein wherein the pharmaceutical composition further comprises a lubricant.

Suitably, the lubricant is present in the pharmaceutical composition in an amount of at least 0.1% w/w by weight of the total weight of the pharmaceutical composition, such as at least 0.5 or 0.8% w/w.

Suitably, the lubricant is present in the pharmaceutical composition in an amount of between about 0.1% w/w and about 2% w/w by weight of the total weight of the pharmaceutical composition, such as between about 0.5% w/w and about 1.5% w/w.

Suitably, the lubricant is present in the pharmaceutical composition in an amount of about 1% w/w by weight of the total weight of the pharmaceutical composition.

Suitably, there is provided a pharmaceutical composition for oral use comprising the plurality of granules as defined herein wherein the plurality of granules further comprise a lubricant.

Suitably, the lubricant is present in the plurality of granules in an amount of at least 0.1% w/w by weight of the total weight of the plurality of granules, such as at least 0.5 or 0.8% w/w.

Suitably, the lubricant is present in the plurality of granules in an amount of between about 0.1% w/w and about 2% w/w by weight of the total weight of the plurality of granules, such as between about 0.5% w/w and about 1.5% w/w.

Suitably, the lubricant is present in the plurality of granules in an amount of about 1% w/w by weight of the total weight of the plurality of granules.

Suitably, the pharmaceutical composition comprises a plurality of granules as herein defined and a plurality of placebo granules.

The plurality of placebo granules comprise a binder and diluent as defined herein for the plurality of granules. The plurality of placebo granules further comprise a disintegrant as defined herein for the plurality of granules. The plurality of placebo granules may further comprise a lubricant as defined herein for the plurality of granules.

Suitably, the plurality of placebo granules are the plurality of granules without the active substance present.

Suitably, the ratio of the plurality of granules to the plurality of placebo granules in the pharmaceutical composition is between 1:10 to 10:1, suitably between 1:5 to 10:1 or between 1:3 to 7:1.

Suitably, the pharmaceutical composition comprises a plurality of granules wherein the plurality of granules comprise:
a. less than 0.5% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 70% w/w of a diluent; and
c. at least 0.5% w/w of a binder;
   wherein weights are relative to the total weight of the plurality of granules.

Suitably, the pharmaceutical composition comprises a plurality of granules wherein the plurality of granules comprise:
a. less than 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 70% w/w of a diluent;
c. at least 0.5% w/w of a binder; and
d. at least 1% w/w of a disintegrant;
   wherein weights are relative to the total weight of the plurality of granules.

Suitably, the pharmaceutical composition comprises a plurality of granules wherein the plurality of granules comprise:
a. less than 0.13% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 80% w/w of a diluent;
c. at least 1% w/w of a binder; and
d. at least 1% w/w of a disintegrant;
   wherein weights are relative to the total weight of the plurality of granules.

Suitably, the pharmaceutical composition comprises:
a. less than 0.11% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 80% w/w of a diluent;
c. at least 1% w/w of a binder; and
d. at least 1% w/w of a disintegrant;
   wherein weights are relative to the total weight of the plurality of granules.

Suitably, the pharmaceutical composition comprises a plurality of granules wherein the plurality of granules comprise:
a. less than 0.05% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 90% w/w of a diluent;
c. at least 1% w/w of a binder; and
d. at least 1% w/w of a disintegrant;
   wherein weights are relative to the total weight of the plurality of granules.

Suitably, the pharmaceutical composition comprises a plurality of granules wherein the plurality of granules comprise:
a. less than 0.13% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 80% w/w of a diluent;
c. at least 1% w/w of a binder;
d. at least 1% w/w of a disintegrant; and
e. at least 0.5% w/w of a lubricant;
   wherein weights are relative to the total weight of the plurality of granules.

Suitably, the pharmaceutical composition comprises a plurality of granules wherein the plurality of granules comprise:
a. less than 0.11% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 80% w/w of a diluent;
c. at least 1% w/w of a binder;
d. at least 1% w/w of a disintegrant; and
e. at least 0.5% w/w of a lubricant;
   wherein weights are relative to the total weight of the plurality of granules.

Suitably, the pharmaceutical composition comprises a plurality of granules wherein the plurality of granules comprise:
a. less than 0.05% w/w of N-(2-(tert-butyl)-1-((4,4-difluorocyclohexyl)methyl)-1H-benzimidazol-5-yl)ethanesulfonamide, or a pharmaceutically acceptable salt thereof;
b. at least 90% w/w of a diluent;
c. at least 1% w/w of a binder;
d. at least 1% w/w of a disintegrant; and
e. at least 0.5% w/w of a lubricant;
   wherein weights are relative to the total weight of the plurality of granules.

Suitably, the pharmaceutical composition comprises a plurality of granules wherein the plurality of granules comprise:
a. between about 0.01% w/w and about 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. between about 85% w/w and about 98% w/w of a diluent;
c. between about 2% w/w and about 3% of a binder;
d. between about 2% w/w and about 6% w/w of a disintegrant; and
e. between about 0.5% w/w and about 1.5% w/w of a lubricant;
   wherein weights are relative to the total weight of the plurality of granules.

Suitably, the pharmaceutical composition comprises a plurality of granules wherein the plurality of granules comprise:
a. about 0.04% w/w of N-(2-(tert-butyl)-1-((4,4-difluorocyclohexyl)methyl)-1H-benzimidazol-5-yl)ethanesulfonamide, or a pharmaceutically acceptable salt thereof;
b. about 93% w/w of a diluent;
c. about 2.5% w/w of a binder;
d. about 4% w/w of a disintegrant; and
e. about 1% w/w of a lubricant;
   wherein weights are relative to the total weight of the plurality of granules.

Suitably, the pharmaceutical composition comprises a plurality of granules wherein the plurality of granules comprise:
a. about 0.1% w/w of N-(2-(tert-butyl)-1-((4,4-difluorocyclohexyl)methyl)-1H-benzimidazol-5-yl)ethanesulfonamide, or a pharmaceutically acceptable salt thereof;
b. about 92% w/w of a diluent;
c. about 2.5% w/w of a binder;
d. about 4% w/w of a disintegrant; and
e. about 1% w/w of a lubricant;
   wherein weights are relative to the total weight of the plurality of granules.

Suitably, the pharmaceutical composition comprises a plurality of granules wherein the plurality of granules comprise:
a. N-(2-(tert-butyl)-1-((4,4-difluorocyclohexyl)methyl)-1H-benzimidazol-5-yl)ethanesulfonamide, or a pharmaceutically acceptable salt thereof;
b. mannitol;
c. hydroxypropyl methyl cellulose; and
d. low-substituted hydroxy propyl cellulose.

Suitably, the pharmaceutical composition comprises a plurality of granules wherein the plurality of granules comprise:
a. N-(2-(tert-butyl)-1-((4,4-difluorocyclohexyl)methyl)-1H-benzimidazol-5-yl)ethanesulfonamide, or a pharmaceutically acceptable salt thereof;
b. mannitol;
c. hydroxypropyl methyl cellulose;
d. low-substituted hydroxy propyl cellulose; and
e. sodium stearyl fumarate.

Suitably, the pharmaceutical composition comprises a plurality of granules wherein the plurality of granules comprise:
a. between about 0.01% w/w and about 0.15% w/w of N-(2-(tert-butyl)-1-((4,4-difluorocyclohexyl)methyl)-1H-benzimidazol-5-yl)ethanesulfonamide, or a pharmaceutically acceptable salt thereof;
b. between about 85% w/w and about 98% w/w of mannitol;
c. between about 2% w/w and about 3% of hydroxypropyl methyl cellulose;
d. between about 2% w/w and about 6% w/w of low-substituted hydroxy propyl cellulose; and
e. between about 0.5% w/w and about 1.5% w/w of sodium stearyl fumarate;
   wherein weights are relative to the total weight of the plurality of granules.

### Diluent

Diluents fill out the size of a granule/composition, making it practical to produce and convenient for the consumer to use.

Suitable diluents include, but are not limited to, calcium carbonate, calcium phosphate, dibasic calcium phosphate, tribasic calcium sulfate, calcium carboxymethylcellulose, dextrin derivatives, dextrin, dextrose, fructose, lactitol, lactose (e.g. anhydrous lactose, spray-dried lactose, α-lactose, β-lactose, Tablettose®, various grades of Pharmatose®, Microtose® or Fast-Floc®), lactose monohydratemannitol, methylcellulose polymers such as, e.g., Methocel A®, Methocel A4C®, Methocel A 15C®, Methocel A4M®), hydroxyethylcellulose, hydroxypropylcellulose, L-hydroxypropylycellulose (low substituted), hydroxypropyl methylcellulose (HPMC) (e.g. Methocel E®, F and K, Metolose SH® of Shin-Etsu, grades of Methocel F® and Metolose 65 SH®, the 4,000, 15,000 and 100,000 cps grades of Methocel K®; and the 4,000, 15,000, 39,000 and 100,000 grades of Metolose 90 SH®), sodium carboxymethylcellulose, carboxymethylene, carboxymethylhydroxyethylcellulose and other cellulose derivatives, starches or modified starches (including potato starch, wheat starch, corn starch, rice starch, pregelatinized maize starch), magnesium carbonate, magnesium oxide, maltitol, maltodextrins, maltose, mannitol, kaolin, sorbitol, starch, sucrose, sugar, xylitol, erythritol, and isomalt, and mixtures thereof and the like.

Preferably, the diluent is mannitol.

In one embodiment, the diluent is not micro crystalline cellulose.

### Binder

A binder is used to impart cohesive qualities and thus facilitates granules being formed during the granulation process and remaining intact afterwards.

Examples of binders include, but not limited to, hydroxy methyl cellulose, hydroxypropyl cellulose, starch (including corn starch and pregelatinized starch), gelatin, sugars (including sucrose, glucose, dextrose, lactose, and sorbitol), waxes, polyethylene glycol, natural and synthetic gums (e.g. acacia, tragacanth sodium alginate, celluloses, and Veegum), and synthetic polymers such as polymethacrylates and polyvinylpyrrolidone (povidone), ethylcellulose, hydroxyethyl cellulose, polyethylene oxide, carboxymethylcellulose, mannitol, methylcellulose, isomalt, polyvinyl alcohol, and mixtures thereof and the like.

Preferably, the binder is hydroxypropyl methyl cellulose or povidone. More preferably, the binder is hydroxypropyl methyl cellulose.

Suitably, the hydroxypropyl methyl cellulose is 6 cps grade hydroxypropyl methyl cellulose.

### Disintegrant

A disintegrant is a substance which helps the composition break up once ingested.

Disintegrants are, but not limited to, cross linked polyvinylpyrolidone (crospovidone, polyplyplasdone XL®, kollidon CL®); starches such as maize starch and dried sodium starch glycolate; gums such as maize starch and dried sodium starch glycolate; gums such as alginic acid, sodium alginate, guar gum; croscarmellose sodium; microfine cellulose, low-substituted hydroxypropylcellulose, carboxymethylcellulose, sodium bicarbonate, and soy polysaccharides, and mixtures thereof and the like.

Preferably, the disintegrant is selected from low-substituted hydroxypropyl cellulose, sodium starch glycolate and croscarmellose sodium. More preferably, the disintegrant is low-substituted hydroxypropyl cellulose.

Low substituted hydroxypropyl cellulose is as defined in the US monograph. For example, when dried at 105° C. for 1 hour, it contains not less than 5.0 percent and not more than 16.0 percent of hydroxypropoxy groups (—OCH₂CHOHCH₃).

### Lubricant

Lubricants prevent composition ingredients from clumping together and from sticking to the tablet punches or capsule filling machine and improve flowability of the composition mixture.

Lubricants are, but not limited to sodium oleate, sodium stearate, sodium benzoate, sodium stearate, sodium chloride, stearic acid, sodium stearyl fumarate, calcium stearate, magnesium lauryl sulfate, sodium stearyl fumarate, sucrose esters or fatty acid, zinc, polyethylene glycol, talc, and glyceryl behenate, and mixtures thereof and the like.

Preferably, the lubricant is sodium stearyl fumarate.

In one embodiment, the lubricant is not magnesium stearate.

### Additional Excipients in the Pharmaceutical Composition

Optionally, additional excipients may be included in the plurality of granules or the pharmaceutical composition according to the present disclosure.

Suitably, the plurality of granules and/or the pharmaceutical composition further comprises a colouring, sweetening flavouring, and/or a preservatives agent such as an antioxidant.

Suitably, the antioxidant comprises butylated hydroxyanisole, butylated hydroxytoluene, ascorbic acid, or alpha tocopherol.

### Content Uniformity of the Active Substance in the Pharmaceutical Composition

Advantageously, the pharmaceutical composition has a low acceptance value for content uniformity. This ensures that the required amount of the active substance is present within each pharmaceutical composition unit and thereby ensures, that upon dosing of the pharmaceutical composition to a patient, the intended dose is administered. This is critical from a patient safety and efficacy perspective.

Suitably, the pharmaceutical composition as herein defined, when subjected to a content uniformity test as described in the European Pharmacopeia, the acceptance value is less than 15.

Suitably, the content uniformity test comprises a chromatographic assay of the pharmaceutical composition. Suitably, the chromatographic assay comprises HPLC. HPLC in this context includes UPLC.

Advantageously, the active substance in the pharmaceutical composition is uniformly distributed and meets the regulatory requirements. Thereby ensuring the intended quantity of the active substance is present in each pharmaceutical composition dosage unit. This is critical from a patient safety and efficacy perspective.

Suitably, the European Pharmacopeia content uniformity test is 2.9.40.

Suitably, the content uniformity test comprises determining the amount of active substance in the pharmaceutical composition. Suitably, the amount of active substance in the pharmaceutical composition is determined using a chromatographic method. Suitably, the chromatographic method is a HPLC method. Suitably, the HPLC method comprises a UV detector. Suitably, the UV detector measures the assay of the active substance at about 217 nm.

Suitably, the HPLC method is a reverse phase HPLC method. Suitably, the reverse phase HPLC method comprises two eluents wherein the ratio of the two eluents changes during the course of the HPLC analysis. Suitably, the first eluent comprises water and the second eluent comprises acetonitrile. Suitably, the first eluent comprises water and phosphoric acid and the second eluent comprises acetonitrile. Suitably, the first eluent comprises water and 0.1% phosphoric acid and the second eluent comprises acetonitrile.

Suitably, the reverse phase HPLC method comprises a HPLC column. Suitably, the HPLC column comprises a C18 stationary phase. Suitably, the HPLC column is at about 40° C. Suitably, the eluents flow through the HPLC column at about 0.5 mL/min.

Suitably, the chromatographic method is a reverse phase HPLC method wherein the reverse phase HPLC method comprises:
a. a UV detector;
b. a HPLC column; and
c. two eluents wherein the ratio of the two eluents changes during the course of the HPLC analysis.

Suitably, the pharmaceutical composition is added to a diluent and the active substance is dissolved in the diluent. Suitably, the diluent is 90:10 v/v 0.1M HCl:acetonitrile.

Suitably, the acceptance value is determined using the following formula:

$$AV = \vert M - X \vert + ks$$

wherein:
X=Mean of individual contents (x₁, x₂ . . . x₃) expressed as a percentage of the label claim;
k=acceptability constant. If n=10 then k=2.4, if n=30 then 2.0 where n=sample size;
s=Sample standard deviation;
M=Reference Value wherein:
for case 1 when T≤101.5:
   If 98.5%≤X≤101.5% then M=X (therefore AV=ks)
   If X<98.5% then M=98.5% (therefore AV=98.5−X+ks)
   If X>101.5%, then M=101.5% (therefore AV=X−101.5+ks)
for case 2 when T>101.5:
   If 98.5%≤X≤T then M=X (therefore AV=ks)
   If X<98.5% then M=98.5% (therefore AV=98.5−X+ks)
   If X>T, then M=T % (therefore AV=X−T+ks); and
T=target test sample amount at time of manufacture.
   Release Rate of the Active Substance from the Pharmaceutical Composition

Suitably, the pharmaceutical composition is an immediate release pharmaceutical composition.

Suitably, at least about 75% of the total amount of the active substance contained in the pharmaceutical composition is released within about the first 45 minutes when the pharmaceutical composition is subjected to an in vitro dissolution test employing 0.1 M HCl or pH 6.8 phosphate buffer as the dissolution medium and the dissolution profile is determined at about 37° C. using a rotation speed of about 50 rpm. Suitably, at least 75% of the total amount of the active substance contained in the pharmaceutical composition is released within the first 45 minutes when the pharmaceutical composition is subjected to an in vitro dissolution test employing 0.1 M HCl or pH 6.8 phosphate buffer as the dissolution medium and the dissolution profile is determined at 37° C. using a rotation speed of 50 rpm.

Advantageously, such a release profile provides optimal absorption of the active substance when administered to a patient.

Suitably, at least about 75% of the total amount of the active substance contained in the pharmaceutical composition is released within about the first 30 minutes when the pharmaceutical composition is subjected to an in vitro dissolution test employing 0.1 M HCl or pH 6.8 phosphate buffer as the dissolution medium and the dissolution profile is determined at about 37° C. using a rotation speed of about 50 rpm. Suitably, at least 75% of the total amount of the active substance contained in the pharmaceutical composition is released within the first 30 minutes when the pharmaceutical composition is subjected to an in vitro dissolution test employing 0.1 M HCl or pH 6.8 phosphate buffer as the dissolution medium and the dissolution profile is determined at 37° C. using a rotation speed of 50 rpm.

Suitably, at least about 85% of the total amount of the active substance contained in the pharmaceutical composition is released within about the first 15 minutes when the pharmaceutical composition is subjected to an in vitro dissolution test employing 0.1 M HCl or pH 6.8 phosphate buffer as the dissolution medium and the dissolution profile is determined at about 37° C. using a rotation speed of about 50 rpm. Suitably, at least 85% of the total amount of the active substance contained in the pharmaceutical composition is released within the first 15 minutes when the pharmaceutical composition is subjected to an in vitro dissolution test employing 0.1 M HCl or pH 6.8 phosphate buffer as the dissolution medium and the dissolution profile is determined at 37° C. using a rotation speed of 50 rpm.

Suitably, the in vitro dissolution test comprises a vessel filled with about 500 mL of dissolution medium wherein the dissolution medium is 0.1 M HCl or pH 6.8 phosphate buffer.

Suitably, the in vitro dissolution test is determined as described in the United States Pharmacopoeia.

Suitably, the in vitro dissolution test comprises a vessel filled with about 500 mL of dissolution medium wherein the dissolution medium is 0.1 M HCl or pH 6.8 phosphate buffer and the dissolution profile is determined at about 37° C. using a rotation speed of about 50 rpm for about the first 45 minutes of the test and then about 200 rpm after about 45 minutes.

Suitably, the in vitro dissolution test further comprises sinkers for the pharmaceutical composition. Suitably, the sinkers are O-ring sinkers.

Suitably, the in vitro dissolution test comprises a detector. Suitably the detector comprises a UV detector.

Suitably, the total amount of the active substance released from the pharmaceutical composition when the pharmaceutical composition is subjected to an in vitro dissolution profile is determined by a chromatographic method or UV spectroscopy. Suitably, the chromatographic method is a HPLC method. Suitably the HPLC method comprises a UV detector. Suitably, the UV detector measures the assay of the active substance at about 217 nm.

Suitably, the HPLC method is a reverse phase HPLC method. Suitably, the reverse phase HPLC method comprises two eluents wherein the ratio of the two eluents changes during the course of the HPLC analysis. Suitably, the first eluent comprises water and the second eluent comprises acetonitrile. Suitably, the first eluent comprises water and phosphoric acid and the second eluent comprises acetonitrile. Suitably, the first eluent comprises water and 0.1% phosphoric acid and the second eluent comprises acetonitrile.

Suitably, the reverse phase HPLC method comprises a HPLC column. Suitably, the HPLC column comprises a C18 stationary phase. Suitably, the HPLC column temperature is at about 40° C. Suitably, the eluents flow through the HPLC column at about 0.5 mL/min.

Suitably, the chromatographic method is a reverse phase HPLC method wherein the reverse phase HPLC method comprises:
a. a UV detector;
b. a HPLC column; and
c. two eluents wherein the ratio of the two eluents changes during the course of the HPLC analysis.

### Stability of the Plurality of Granules and Pharmaceutical Composition

Suitably, the active substance is stable in the plurality of granules and/or the pharmaceutical composition.

Preferably, the active substance is stable within the plurality of granules and/or the pharmaceutical composition during the lifetime of the plurality of granules and/or pharmaceutical composition, for example during the manufacture of the plurality of granules and/or pharmaceutical composition and preferably at least until the pharmaceutical composition is administered to a patient. Stability is an important safety aspect of a pharmaceutical product (input material, i.e., plurality of granules, and the pharmaceutical composition). Having stability throughout the manufacturing process to form the product (granules and composition) means the intended dose of the active substance is present in the manufactured product and degradation products of the active substance being minimised. Furthermore, a stable product results in the assay of the active substance being consistent over time and degradation products at a minimal level. These factors allow for consistent dosing of the active substance to a patient, and therefore result in the desired dose being administered to the patient. Furthermore, the end user, for example the patient, is not administered degradation products of the active substance. Stability of the active substance within the pharmaceutical composition is important from a patient safety and efficacy perspective.

Suitably, the active substance is stable during the process to prepare the plurality of granules and/or the pharmaceutical composition.

Suitably, the assay of the active substance in the plurality of granules after manufacturing is about 90 to about 110% of nominal, such as about 95 to about 105% or about 98 to about 102%, as determined by a chromatographic method. Suitably, the assay of the active substance in the pharmaceutical composition after manufacturing is about 90 to about 110% of nominal, such as about 95 to about 105% or about 98 to about 102%, as determined by a chromatographic method.

Suitably, the total amount of degradation products of the active substance in the plurality of granules after manufacturing is less than or equal to about 4 area %, such as less than or equal to 3, 2 or 1 area %, as determined by a chromatographic method. Suitably, the total amount of degradation products of the active substance in the pharmaceutical composition after manufacturing is less than or equal to about 4 area %, such as less than or equal to 3, 2 or 1 area %, as determined by a chromatographic method.

Suitably, the active substance is stable in the plurality of granules and/or the pharmaceutical composition for at least about 2 weeks, about 1 month, about 2 months, about 3 months, about 6 months, about 12 months, or about 24 months. Suitably, the active substance is stable in the plurality of granules and/or the pharmaceutical composition for at least 2 weeks, 1, 2, 3, 6, 12, or 24 months.

Suitably, the active substance is chemically stable in the plurality of granules and/or the pharmaceutical composition for at least about 2 weeks, about 1 month, about 2 months, about 3 months, about 6 months, about 12 months, or about 24 months. Suitably, the active substance is chemically stable in the plurality of granules and/or the pharmaceutical composition for at least 2 weeks, 1, 2, 3, 6, 12, or 24 months.

Suitably, the active substance is stable in the plurality of granules for at least about 2 weeks, about 1 month, about 2 months, about 3 months, about 6 months, about 12 months, or about 24 months when the plurality of granules are stored at 25° C./60% RH and/or 40° C./75% RH. Suitably, the active substance is stable in the plurality of granules for at least 2 weeks, 1, 2, 3, 6, 12, or 24 months when the plurality of granules are stored at 25° C./60% RH and/or 40° C./75% RH.

Suitably, the active substance is stable in the pharmaceutical composition for at least about 2 weeks, about 1 month, about 2 months, about 3 months, about 6 months, about 12 months, or about 24 months when the pharmaceutical composition is stored at 25° C./60% RH and/or 40° C./75% RH. Suitably, the active substance is stable in the pharmaceutical composition for at least 2 weeks, 1, 2, 3, 6, 12, or 24 months when the pharmaceutical composition is stored at 25° C./60% RH and/or 40° C./75% RH.

Suitably, the total amount of degradation products of the active substance is less than or equal to about 4 area %, such as less than or equal to 3, 2 or 1 area %, as determined by a chromatographic method when the plurality of granules are stored at 25° C./60% RH and/or 40° C./75% RH for at least about 2 weeks, about 1 month, about 2 months, about 3 months, about 6 months, about 12 months, or about 24 months. Suitably, the total amount of degradation products of the active substance is less than or equal to 4 area %, such as less than or equal to 3, 2 or 1 area %, as determined by a chromatographic method when the plurality of granules are stored at 25° C./60% RH and/or 40° C./75% RH for 2 weeks, 1, 2, 3, 6, 12, or 24 months.

Suitably, the total amount of degradation products of the active substance is less than or equal to about 4 area %, such as less than or equal to 3, 2 or 1 area %, as determined by a chromatographic method when the pharmaceutical composition is stored at 25° C./60% RH and/or 40° C./75% RH for at least about 2 weeks, about 1 month, about 2 months, about 3 months, about 6 months, about 12 months, or about 24 months. Suitably, the total amount of degradation products of the active substance is less than or equal to 4 area %, such as less than or equal to 3, 2 or 1 area %, as determined by a chromatographic method when the pharmaceutical composition is stored at 25° C./60% RH and/or 40° C./75% RH for 2 weeks, 1, 2, 3, 6, 12, or 24 months.

A degradation product of the active substance is an impurity which forms upon storage of the active substance (or during manufacture of the pharmaceutical composition). The degradation product may be formed from exposure of the active substance to water, oxygen, peroxides, or UV light.

Suitably, the assay of the active substance is about 90 to about 110% of nominal, such as about 95 to about 105% or about 98 to about 102%, as determined by a chromatographic method when the plurality of granules are stored at 25° C./60% RH and/or 40° C./75% RH for at least about 2 weeks, about 1 month, about 2 months, about 3 months, about 6 months, about 12 months, or about 24 months. Suitably, the assay of the active substance is 90 to 110% of nominal, such as about 95 to about 105% or about 98 to about 102%, as determined by a chromatographic method when the plurality of granules are stored at 25° C./60% RH and/or 40° C./75% RH for 2 weeks, 1, 2, 3, 6, 12, or 24 months.

Suitably, the assay of the active substance is about 90 to about 110% of nominal, such as about 95 to about 105% or about 98 to about 102%, as determined by a chromatographic method when the pharmaceutical composition is stored at 25° C./60% RH and/or 40° C./75% RH for at least about 2 weeks, about 1 month, about 2 months, about 3 months, about 6 months, about 12 months, or about 24 months. Suitably, the assay of the active substance is 90 to 110% of nominal, such as about 95 to about 105% or about 98 to about 102%, as determined by a chromatographic method when the pharmaceutical composition is stored at 25° C./60% RH and/or 40° C./75% RH for 2 weeks, 1, 2, 3, 6, 12, or 24 months.

Suitably, the total amount of degradation products of the active substance is less than or equal to about 4 area % as determined by a chromatographic method and the assay of the active substance is about 90 to about 110% of nominal as determined by a chromatographic method when the plurality of granules are stored at 25° C./60% RH and/or 40° C./75% RH for at least about 2 weeks, about 1 month, about 2 months, about 3 months, about 6 months, about 12 months, or about 24 months. Suitably, the total amount of degradation products of the active substance is less than or equal to 4 area % and the assay of the active substance is 90 to 110% of nominal as determined by a chromatographic method when the plurality of granules are stored at 25° C./60% RH and/or 40° C./75% RH for 2 weeks, 1, 2, 3, 6, 12, or 24 months.

Suitably, the total amount of degradation products of the active substance is less than or equal to about 4 area % as determined by a chromatographic method and the assay of the active substance is about 90 to about 110% of nominal as determined by a chromatographic method when the pharmaceutical composition is stored at 25° C./60% RH and/or 40° C./75% RH for at least about 2 weeks, about 1 month, about 2 months, about 3 months, about 6 months, about 12 months, or about 24 months. Suitably, the total amount of degradation products of the active substance is less than or equal to 4 area % and the assay of the active substance is 90 to 110% of nominal as determined by a chromatographic method when the pharmaceutical composition is stored at 25° C./60% RH and/or 40° C./75% RH for 2 weeks, 1, 2, 3, 6, 12, or 24 months.

Suitably, the active substance is stable in the plurality of granules for at least about 2 weeks, about 1 month, about 2 months, about 3 months, about 6 months, about 12 months, or about 24 months when the plurality of granules are protected from light, suitably UV radiation. Suitably, the active substance is stable in the plurality of granules for at least 2 weeks, 1, 2, 3, 6, 12, or 24 months when the plurality of granules are protected from light, suitably UV radiation.

Suitably, the active substance is stable in the pharmaceutical composition for at least about 2 weeks, about 1 month, about 2 months, about 3 months, about 6 months, about 12 months, or about 24 months when the pharmaceutical composition is protected from light, suitably UV radiation. Suitably, the active substance is stable in the pharmaceutical composition for at least 2 weeks, 1, 2, 3, 6, 12, or 24 months when the pharmaceutical composition is protected from light, suitably UV radiation.

Suitably, the chromatographic method is a HPLC method. HPLC in this context includes UPLC.

Suitably the HPLC method comprises a UV detector. Suitably, the UV detector measures the assay and/or degradation products of the active substance at about 256 nm.

Suitably, the HPLC method is a reverse phase HPLC method. Suitably, the reverse phase HPLC method comprises two eluents wherein the ratio of the two eluents changes during the course of the HPLC analysis. Suitably, the first eluent comprises water and the second eluent comprises acetonitrile. Suitably, the first eluent comprises water and ammonium acetate and the second eluent comprises acetonitrile. Suitably, the first eluent comprises water and 10 mM ammonium acetate and the second eluent comprises acetonitrile.

Suitably, the reverse phase HPLC method comprises a HPLC column. Suitably, the HPLC column comprises a C18 stationary phase. Suitably, the HPLC column temperature is at about 40° C.

Suitably, the eluents flow through the HPLC column at about 0.3 mL/min.

Suitably, the chromatographic method is a reverse phase HPLC method wherein the reverse phase HPLC method comprises:
a. a UV detector;
b. a HPLC column; and
c. two eluents wherein the ratio of the two eluents changes during the course of the HPLC analysis.

### Process of Manufacture

Suitably, the plurality of granules are prepared using a wet granulation method. Using a wet granulation method to prepare the plurality of granules helps to ensure the active substance is uniformly distributed throughout the granules, which in turn ensures the correct dose of the active substance is present in the pharmaceutical composition.

In a further aspect, there is provided a process to a plurality of granules, wherein the process comprises:
a. mixing a binder and diluent together;
b. adding a primary granulation fluid to the mixture from step a. and mixing until granules are formed; and
c. drying the mixture from step b;
   wherein N-(2-(tert-butyl)-1-((4,4-difluorocyclohexyl)methyl)-1H-benzimidazol-5-yl)ethanesulfonamide, or a pharmaceutically acceptable salt thereof, is either mixed with the binder and diluent in step a. or dissolved in the primary granulation fluid.

Suitably, N-(2-(tert-butyl)-1-((4,4-difluorocyclohexyl)methyl)-1H-benzimidazol-5-yl)ethanesulfonamide, or a pharmaceutically acceptable salt thereof, is mixed with the binder and diluent in step a.

Suitably, N-(2-(tert-butyl)-1-((4,4-difluorocyclohexyl)methyl)-1H-benzimidazol-5-yl)ethanesulfonamide, or a pharmaceutically acceptable salt thereof, is dissolved in the primary granulation fluid.

Suitably, step a. further comprises mixing a disintegrant together with the binder and diluent

Suitably, N-(2-(tert-butyl)-1-((4,4-difluorocyclohexyl)methyl)-1H-benzimidazol-5-yl)ethanesulfonamide, or a pharmaceutically acceptable salt thereof, has a solubility of at least 0.6 mg/mL in the primary granulation fluid at room temperature, such as at least 7 mg/mL.

Suitably, the primary granulation fluid is selected from ethanol, 0.1 M HCl, pH 3 citric acid buffer and 5% aqueous SLS. Advantageously, the active substance is stable in these primary granulation fluids and has a solubility of >0.6 mg/mL.

Preferably, the primary granulation fluid is 0.1 M HCl.

Suitably, the primary granulation fluid is added at a rate of about 2 to about 10 mL/min, preferably about 4 to about 7 mL/min, more preferably about 5 to about 6 mL/min in step b.

Suitably, the mixture is mixed at high speed in step b.

Suitably, the granules are dried at an elevated temperature in step c. Suitably, the elevated temperature is greater than 30° C., such as greater than 40° C., greater than 50° C.

Suitably, the process further comprises adding a secondary granulation fluid to step b. Suitably, the secondary granulation fluid selected from ethanol, 0.1 M HCl, pH 3 citric acid buffer and 5% aqueous SLS. Preferably, the secondary granulation fluid is 0.1 M HCl.

Suitably, the secondary fluid is added over a period of at least 1 minute, such as at least 2 minutes, at least 5 minutes or at least 8 minutes.

### Therapeutic Uses and Applications

The present disclosure provides a pharmaceutical composition as defined herein for use as a medicament.

The present disclosure provides a pharmaceutical composition as defined herein for use in therapy.

The present disclosure provides use of a pharmaceutical composition as defined herein for use in the manufacture of a medicament.

The present disclosure provides a pharmaceutical composition as defined herein for use in a disease state or disorder in which dysfunction of CB₁ and/or CB₂ receptors are present or implicated.

The present disclosure provides a method for treating a disease state or disorder in which dysfunction of CB₁ and/or CB₂ receptors are present or implicated, said method comprising administering to said patient a therapeutically effective amount of a pharmaceutical composition as defined herein.

The present disclosure provides a use of a pharmaceutical composition as defined herein for use in the manufacture of a medicament for use in a disease state or disorder in which dysfunction of CB₁ and/or CB₂ receptors are present or implicated.

The present disclosure provides a pharmaceutical composition for use in the treatment of an anorexia associated condition; a cachexia associated condition; or anorexia nervosa.

The present disclosure provides a method of treating an anorexia associated condition, a cachexia associated condition or anorexia nervosa, said method comprising administering to said patient a therapeutically effective amount of a pharmaceutical composition as defined herein.

The present disclosure provides use of a pharmaceutical composition as defined herein for use in the manufacture of a medicament for the treatment of an anorexia associated condition; a cachexia associated conditions; or anorexia nervosa.

Suitably, the anorexia associated condition is anorexia associated with cancer, anorexia associated with HIV (Human Immunodeficiency Virus), anorexia associated with Chronic Kidney Disease, anorexia associated with dementia, or anorexia associated with chronic congestive heart failure.

Suitably, the anorexia associated condition is anorexia associated with cancer.

Suitably, the cachexia associated condition is cachexia associated with cancer, cachexia associated with HIV, cachexia associated with Chronic Kidney Disease, cachexia associated with dementia, or cachexia associated with chronic congestive heart failure.

The present disclosure provides a pharmaceutical composition as defined herein for use in the treatment of a pain condition.

The present disclosure provides a method of treating a pain condition in a patient in need of such treatment, said method comprising administering to said patient a therapeutically effective amount of a pharmaceutical composition as defined herein.

The present disclosure provides use of a pharmaceutical composition as defined herein for use in the manufacture of a medicament for the treatment of a pain condition.

Suitably, the pain condition is a pain condition in cancer, acute pain, chronic pain, neuropathic pain, back pain, cancer pain, visceral pain, pain caused by rheumatoid arthritis, migraine. Suitably, the pain condition in cancer is a pain condition in cancer associated with the reduction of opiate use, associated with the reduction in nausea and/or vomiting in cancer patients.

The present disclosure provides a pharmaceutical composition as defined herein for use in the treatment of anxiety disorders, cancer, multiple sclerosis, Parkinson's disease, Huntington's chorea, Alzheimer's disease, AIDS (Acquired Immune Deficiency Syndrome), amyotrophic lateral sclerosis, gastrointestinal disorders, cardiovascular disorders, or insomnia.

The present disclosure provides a method of treating anxiety disorders, cancer, multiple sclerosis, Parkinson's disease, Huntington's chorea, Alzheimer's disease, AIDS, amyotrophic lateral sclerosis, gastrointestinal disorders, cardiovascular disorders, or insomnia, said method comprising administering to said patient a therapeutically effective amount of a pharmaceutical composition as defined herein.

The present disclosure provides use of a pharmaceutical composition as defined herein for use in the manufacture of a medicament for the treatment of anxiety disorders, cancer, multiple sclerosis, Parkinson's disease, Huntington's chorea, Alzheimer's disease, AIDS, amyotrophic lateral sclerosis, gastrointestinal disorders, cardiovascular disorders, or insomnia.

The present disclosure provides a pharmaceutical composition as defined herein for use as an immunomodulator. Suitably, the pharmaceutical composition as defined herein is for use in the treatment of autoimmune diseases such as arthritis, collagen diseases, or allergies. Suitably, the pharmaceutical composition as defined herein is for use in skin graft therapy, organ transplant therapy and other surgical needs. Suitably, the pharmaceutical composition as defined herein is for use as an anti-tumor agent or an anti-viral agent.

The present disclosure provides a method of treating autoimmune diseases such as arthritis, collagen diseases, or allergies, said method comprising administering to said patient a therapeutically effective amount of a pharmaceutical composition. The present disclosure provides a method of treatment in skin graft therapy, organ transplant therapy and other surgical needs, said method comprising administering to said patient a therapeutically effective amount of a pharmaceutical composition as defined herein.

The present disclosure provides use of a pharmaceutical composition as defined herein for use in the manufacture of a medicament for use as an immunomodulator. The present disclosure provides use of a pharmaceutical composition as defined herein for use in the manufacture of a medicament for the treatment of autoimmune diseases such as arthritis, collagen diseases, or allergies. The present disclosure provides use of a pharmaceutical composition as defined herein for use in the manufacture of a medicament for use in skin graft therapy, organ transplant therapy and other surgical needs. The present disclosure provides use of a pharmaceutical composition as defined herein for use in the manufacture of a medicament for use as an anti-tumour agent or an anti-viral agent.

The present disclosure provides a pharmaceutical composition as defined herein for use in the treatment of diarrhoea; depression; anxiety and stress-related disorders; urinary incontinence; premature ejaculation; various mental illnesses; cough; lung oedema; various gastro-intestinal disorders; Parkinson's disease and other motor disorders; traumatic brain injury; stroke; cardioprotection following miocardial infarction; spinal injury and drug addiction including the treatment of alcohol, nicotine, opioid and other drug abuse; and for disorders of the sympathetic nervous system, such as hypertension.

The present disclosure provides a method of treating diarrhoea; depression; anxiety and stress-related disorders; urinary incontinence; premature ejaculation; various mental illnesses; cough; lung oedema; various gastro-intestinal disorders; Parkinson's disease and other motor disorders; traumatic brain injury; stroke; cardioprotection following miocardial infarction; spinal injury and drug addiction including the treatment of alcohol, nicotine, opioid and other drug abuse; and for disorders of the sympathetic nervous system, such as hypertension, said method comprising administering to said patient a therapeutically effective amount of a pharmaceutical composition as defined herein.

The present disclosure provides use of a pharmaceutical composition as defined herein for use in the manufacture of a medicament for use in the treatment of diarrhoea; depression; anxiety and stress-related disorders; urinary incontinence; premature ejaculation; various mental illnesses; cough; lung oedema; various gastro-intestinal disorders; Parkinson's disease and other motor disorders; traumatic brain injury; stroke; cardioprotection following miocardial infarction; spinal injury and drug addiction including the treatment of alcohol, nicotine, opioid and other drug abuse; and for disorders of the sympathetic nervous system, such as hypertension.

Suitably, the anxiety and stress disorders are selected from post-traumatic stress disorders, panic disorder, generalized anxiety disorder, social phobia, and obsessive compulsive disorder.

Suitably, the gastro-intestinal disorder is selected from constipation and functional gastrointestinal disorders such as Irritable Bowel Syndrome and Functional Dyspepsia.

The present disclosure provides a pharmaceutical composition as defined herein for use as an analgesic agent, for example for use during general anaesthesia and monitored anaesthesia care.

The present disclosure provides a method of providing an analgesic effect, for example for use during general anaesthesia and monitored anaesthesia care, said method comprising administering to said patient a therapeutically effective amount of a pharmaceutical composition as defined herein.

The present disclosure provides use of a pharmaceutical composition as defined herein for use in the manufacture of a medicament for use as an analgesic agent, for example for use during general anaesthesia and monitored anaesthesia care.

Combinations of agents with different properties are often used to achieve a balance of effects needed to maintain the anaesthetic state (e.g. amnesia, analgesia, muscle relaxation and sedation). Included in this combination are inhaled anaesthetics, hypnotics, anxiolytics, neuromuscular blockers and opioids.

The present disclosure therefore provides a method of activating the CB₁ and/or CB₂ receptor in vitro or in vivo, said method comprising contacting a cell with an effective amount of a pharmaceutical composition as defined herein.

The present disclosure also provides a method of treating a disease or disorder in which dysfunction of CB₁ and/or CB₂ receptors are implicated in a patient in need of such treatment, said method comprising administering to said patient a therapeutically effective amount of a pharmaceutical composition as defined herein.

A therapeutically effective amount of a pharmaceutical composition comprising the active substance of the present disclosure for use in therapy is an amount to achieve a particular biological or therapeutic result such as, but not limited to, biological or therapeutic results disclosed, described, or exemplified herein. For example, to treat or prevent a disease or condition referred to herein, slow its progression and/or reduce the symptoms associated with the condition and/or disease.

The amount of the active substance that is combined with one or more excipients to produce the pharmaceutical composition will necessarily vary depending upon the individual treated and the particular route of administration. An immediate release composition intended for oral administration to humans will generally contain, for example, from about 10 μg to about 1000 μg of the active substance (for example about 10 μg to about 650 μg) compounded with an appropriate and convenient amount of excipients which may vary from about 0.01 to about 99.99 percent by weight of the total pharmaceutical composition.

The size of the dose for therapeutic or prophylactic purposes of the active substance will naturally vary according to the nature and severity of the conditions, the age and sex of the animal or patient and the route of administration, according to well known principles of medicine.

In one embodiment, between about 25 and about 700 μg/dose/day of Compound I is administered to the patient, such as between about 50 and about 650 μg/dose/day. Suitably, about 50, 150, 250, 400 or 650 μg/dose/day of Compound I is administered to the patient,

### Routes of Administration

The pharmaceutical compositions as defined herein may be administered to a subject by any convenient oral route of administration (e.g. by ingestion).

### Combination Therapies

The pharmaceutical compositions of the present disclosure are useful for the treatment of conditions in which therapy with an agonist of CB1 and/or CB2 is beneficial. Examples of such conditions are outlined above in the therapeutic use section of the present disclosure.

The pharmaceutical compositions of the present disclosure may be used in combination with one or more additional therapeutic agents for the treatment of the condition concerned.

An additional therapeutic agent may be included in the pharmaceutical composition comprising Compound I, or a pharmaceutically acceptable salt thereof, as defined herein, or, alternatively, it may be administered separately, either at the same time as the pharmaceutical composition as defined herein or at an earlier or later time.

Therefore, in a further aspect of the disclosure, there is provided a combination product comprising a pharmaceutical composition as defined herein and a pharmaceutical composition comprising an additional therapeutic agent useful in the treatment or prevention of any one of the therapeutic conditions referred to herein, wherein the pharmaceutical composition as defined herein and the pharmaceutical composition comprising the additional therapeutic agent are administered simultaneously, sequentially or separately.

According to this aspect of the disclosure there is provided a combination for use in the treatment of disease or condition in which therapy with a CB1 and/or CB2 agonist is beneficial (e.g. cancer anorexia), comprising a pharmaceutical composition of the present disclosure as defined hereinbefore, and one or more additional therapeutic agents.

The present disclosure also provides a pharmaceutical composition as defined herein and one or more additional therapeutic agents for use in the treatment of anorexia, especially cancer anorexia, wherein the pharmaceutical composition as defined herein and the additional therapeutic agent are administered simultaneously, sequentially or separately.

In certain embodiments, the pharmaceutical composition as defined herein may be used for the treatment of cancer anorexia in patients undergoing cancer therapy. The cancer therapy may in the form of radiotherapy, surgical therapy, chemotherapy, immunotherapy or a combination thereof.

Herein, where the term “combination” is used it is to be understood that this refers to simultaneous, separate, or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

As indicated above, the combination therapy of the present disclosure may be achieved by way of the simultaneous, sequential, or separate dosing of the individual components of the treatment. Such combination products employ the pharmaceutical compositions as defined herein within the dosage range described hereinbefore and one or more further pharmaceutically-active agents, wherein each additional agent is used within its approved dosage range.

### Particular Aspects and Embodiments of the Present Disclosure

The following numbered paragraphs further define particular aspects and embodiments of the present disclosure.
(1) A plurality of granules comprising:
   a. N-(2-(tert-butyl)-1-((4,4-difluorocyclohexyl)methyl)-1H-benzimidazol-5-yl)ethanesulfonamide, or a pharmaceutically acceptable salt thereof; and
   b. at least one pharmaceutically acceptable excipient.
(2) The plurality of granules according to clause 1, wherein the pharmaceutically acceptable excipient is selected from a diluent, disintegrant and binder.
(3) The plurality of granules according to any preceding clause, wherein the plurality of granules comprise a diluent and a binder.
(4) The plurality of granules according to any preceding clause, wherein N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of less than 0.5% w/w by weight of the total weight of the plurality of granules, such as less than 0.4, 0.3, 0.2, 0.15, 0.14, 0.13, 0.12, 0.11, 0.10, 0.08, 0.06, or 0.05% w/w.
(5) The plurality of granules according to any preceding clause, wherein N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of between about 0.01% w/w and about 0.5% w/w by weight of the total weight of the plurality of granules, such as between about 0.01% w/w and about 0.4% w/w, about 0.01% w/w and about 0.3% w/w, about 0.01% w/w and about 0.2% w/w, about 0.01% w/w and about 0.15% w/w, about 0.01% w/w and about 0.12% w/w, about 0.02% w/w and about 0.12% w/w, or about 0.03% w/w and about 0.10% w/w.
(6) The plurality of granules according to any preceding clause, wherein N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of about 0.04, about 0.10 or about 0.12% w/w of the total weight of the plurality of granules.
(7) The plurality of granules according to clauses (2) to (6), wherein the diluent is present in the plurality of granules in an amount of at least 70% w/w by weight of the total weight of the plurality of granules, such as at least 75, 80, 85, or 90% w/w.
(8) The plurality of granules according to clauses (2) to (7), wherein the diluent is present in the plurality of granules in an amount of between about 75% w/w and about 98% w/w by weight of the total weight of the plurality of granules, such as between about 85% w/w and about 98% w/w or between about 90% w/w and about 95% w/w.
(9) The plurality of granules according to clauses (2) to (8), wherein the diluent is present in the plurality of granules in an amount of about 93% by weight of the total weight of the plurality of granules.
(10) The plurality of granules according to clauses (2) to (9), wherein the binder is present in the plurality of granules in an amount of at least 0.5% w/w by weight of the total weight of the plurality of granules, such as at least 1 or 2% w/w.
(11) The plurality of granules according to clauses (2) to (10), wherein the binder is present in the plurality of granules in an amount between about 0.5% w/w and about 5% w/w by weight of the total weight of the plurality of granules, such as between about 1% w/w and about 4% w/w or between about 2% w/w and about 3% w/w.
(12) The plurality of granules according to clauses (2) to (11), wherein the binder is present in the plurality of granules in an amount of about 2.5% w/w by weight of the total weight of the plurality of granules.
(13) The plurality of granules according to any preceding clause, wherein the plurality of granules comprise a diluent, disintegrant and binder.
(14) The plurality of granules according to clauses (2) to (13), wherein the disintegrant is present in the plurality of granules in an amount of at least 0.5% w/w by weight of the total weight of the plurality of granules, such as at least 1, 2, or 3% w/w.
(15) The plurality of granules according to clauses (2) to (14), wherein the disintegrant is present in the plurality of granules in an amount of between about 1% w/w and about 10% w/w by weight of the total weight of the plurality of granules, such as between about 2% w/w and about 6% w/w or between about 3% w/w and about 5% w/w.
(16) The plurality of granules according to clauses (2) to (14), wherein the disintegrant is present in the plurality of granules in an amount of about 4% w/w of the total weight of the plurality of granules.
(17) The plurality of granules according to clauses (2) to (16), wherein the diluent is selected from calcium carbonate, calcium phosphate, dibasic calcium phosphate, tribasic calcium sulfate, calcium carboxymethylcellulose, dextrin derivatives, dextrin, dextrose, fructose, lactitol, lactose (e.g. anhydrous lactose, spray-dried lactose, α-lactose, β-lactose, Tablettose®, various grades of Pharmatose®, Microtose® or Fast-Floc®), lactose monohydratemannitol, methylcellulose polymers such as, e.g., Methocel A®, Methocel A4C®, Methocel A 15C®, Methocel A4M®), hydroxyethylcellulose, hydroxypropylcellulose, L-hydroxypropylycellulose (low substituted), hydroxypropyl methylcellulose (HPMC) (e.g. Methocel E®, F and K, Metolose SH® of Shin-Etsu, grades of Methocel F® and Metolose 65 SH®, the 4,000, 15,000 and 100,000 cps grades of Methocel K®; and the 4,000, 15,000, 39,000 and 100,000 grades of Metolose 90 SH®), sodium carboxymethylcellulose, carboxymethylene, carboxymethylhydroxyethylcellulose and other cellulose derivatives, starches or modified starches (including potato starch, wheat starch, corn starch, rice starch, pregelatinized maize starch), magnesium carbonate, magnesium oxide, maltitol, maltodextrins, maltose, mannitol, kaolin, sorbitol, starch, sucrose, sugar, xylitol, erythritol, and isomalt, and mixtures thereof and the like.
(18) The plurality of granules according to clause (17), wherein the diluent is mannitol.
(19) The plurality of granules according to clauses (2) to (18), wherein the binder is selected from hydroxy methyl cellulose, hydroxypropyl cellulose, starch (including corn starch and pregelatinized starch), gelatin, sugars (including sucrose, glucose, dextrose, lactose, and sorbitol), waxes, polyethylene glycol, natural and synthetic gums (e.g. acacia, tragacanth sodium alginate, celluloses, and Veegum), and synthetic polymers such as polymethacrylates and polyvinylpyrrolidone (povidone), ethylcellulose, hydroxyethyl cellulose, polyethylene oxide, carboxymethylcellulose, mannitol, methylcellulose, isomalt, polyvinyl alcohol, and mixtures thereof and the like.
(20) The plurality of granules according to clause (19), wherein the binder is hydroxypropyl methyl cellulose or povidone.
(21) The plurality of granules according to clause (20), wherein the binder is hydroxypropyl methyl cellulose.
(22) The plurality of granules according to clause (21), wherein the binder is 6 cps grade hydroxypropyl methyl cellulose.
(23) The plurality of granules according to clauses (2) to (22), wherein the disintegrant is selected from cross linked polyvinylpyrolidone (crospovidone, polyplyplasdone XL®, kollidon CL®); starches such as maize starch and dried sodium starch glycolate; gums such as maize starch and dried sodium starch glycolate; gums such as alginic acid, sodium alginate, guar gum; croscarmellose sodium; microfine cellulose, low-substituted hydroxypropylcellulose, mixtures thereof and the like.
(24) The plurality of granules according to clause (23), wherein the disintegrant is selected from low-substituted hydroxypropyl cellulose, sodium starch glycolate and croscarmellose sodium.
(25) The plurality of granules according to clause (24), wherein the disintegrant is low-substituted hydroxypropyl cellulose.
(26) The plurality of granules according to any preceding clause, wherein the bulk density of the granules is between about 0.1 and about 0.7 g/mL, such as between about 0.2 and about 0.6 g/mL, or between about 0.3 and about 0.5 g/mL.
(27) The plurality of granules according to any preceding clause further comprising a lubricant.
(28) The plurality of granules according to clause (27), wherein the lubricant is selected from sodium oleate, sodium stearate, sodium benzoate, sodium stearate, sodium chloride, stearic acid, sodium stearyl fumarate, calcium stearate, magnesium lauryl sulfate, sodium stearyl fumarate, sucrose esters or fatty acid, zinc, polyethylene glycol, talc, and glyceryl behenate, and mixtures thereof and the like.
(29) The plurality of granules according to clause (28), wherein the lubricant is sodium stearyl fumarate.
(30) The plurality of granules according to any preceding clause, wherein the size of the granules is less than 1200 μm.
(31) The plurality of granules according to any preceding clause, wherein the active substance is stable in the plurality of granules.
(32) The plurality of granules according to any preceding clause, wherein the active substance is stable during the process to prepare the plurality of granules.
(33) The plurality of granules according to clause (32), wherein the assay of the active substance in the plurality of granules after manufacturing is about 90 to about 110% of nominal, such as about 95 to about 105% or about 98 to about 102%, as determined by a chromatographic method.
(34) The plurality of granules according to clauses (32) or (33), wherein the total amount of degradation products of the active substance in the plurality of granules after manufacturing is less than or equal to about 4 area %, such as less than or equal to 3, 2 or 1 area %, as determined by a chromatographic method.
(35) The plurality of granules according to clauses (31) to (34), wherein the active substance is chemically stable in the plurality of granules for at least about 2 weeks, about 1 month, about 2 months, about 3 months, about 6 months, about 12 months, or about 24 months.
(36) The plurality of granules according to clause (35), wherein the total amount of degradation products of the active substance is less than or equal to about 4 area %, such as less than or equal to 3, 2 or 1 area %, as determined by a chromatographic method when the plurality of granules are stored at 25° C./60% RH and/or 40° C./75% RH for at least about 2 weeks, about 1 month, about 2 months, about 3 months, about 6 months, about 12 months, or about 24 months.
(37) The plurality of granules according to clauses (35) or (36), wherein the assay of the active substance is about 90 to about 110% of nominal, such as about 95 to about 105% or about 98 to about 102%, as determined by a chromatographic method when the plurality of granules are stored at 25° C./60% RH and/or 40° C./75% RH for at least about 2 weeks, about 1 month, about 2 months, about 3 months, about 6 months, about 12 months, or about 24 months.
(38) A pharmaceutical composition for oral use comprising a plurality of granules according to clauses (1) to (37).
(39) The pharmaceutical composition according to clause (38), wherein the pharmaceutical composition consists essentially of the plurality of granules of clauses (1) to (37).
(40) The pharmaceutical composition according to clause (38), wherein the pharmaceutical composition consists of the plurality of granules of clauses (1) to (37).
(41) The pharmaceutical composition according to clause (38), wherein the pharmaceutical composition is a capsule or tablet.
(42) The pharmaceutical composition according to clause (41), wherein the pharmaceutical composition is a capsule.
(43) The pharmaceutical composition according to clause (42), wherein the pharmaceutical composition comprises a capsule shell.
(44) The pharmaceutical composition according to clause (43), wherein the capsule shell comprises hydroxypropyl methyl cellulose or gelatin.
(45) The pharmaceutical composition according to clause (44), wherein the capsule shell comprises gelatin, such as hard gelatin.
(46) The pharmaceutical composition according to clauses (43) to (45) wherein the capsule shell is a size 4 size capsule shell.
(47) The pharmaceutical composition according to clauses (38) to (46), wherein the plurality of granules comprise between about 0.001 and about 0.8% w/w of the active substance by weight of the total weight of the plurality of granules, such as between about 0.01 and about 0.8% w/w, about 0.02 and about 0.2% w/w, or about 0.03 and about 0.15% w/w.
(48) The pharmaceutical composition according to clauses (38) to (47), wherein the plurality of granules comprising about 0.1% w/w of the active substance by weight of the total weight of the plurality of granules.
(49) The pharmaceutical composition according to clauses (38) to (47), wherein the plurality of granules comprising about 0.4% w/w of the active substance by weight of the total weight of the plurality of granules.
(50) The pharmaceutical composition according to clauses (38) to (49), wherein the pharmaceutical composition comprises between about 10 μg and about 1000 μg of the active substance, such as between about 10 μg and about 500 μg, about 20 μg and about 500 μg, about 30 μg and about 300 μg, or about 40 μg and about 250 μg.
(51) The pharmaceutical composition according to clauses (38) to (50), wherein the pharmaceutical composition comprises about 50 μg of the active substance.
(52) The pharmaceutical composition according to clauses (38) to (50), wherein the pharmaceutical composition comprises about 200 μg of the active substance.
(53) The pharmaceutical composition according to clauses (38) to (52), wherein the plurality of granules comprise a diluent.
(54) The pharmaceutical composition according to clause (53), wherein the diluent is present in the plurality of granules in an amount of at least 70% w/w by weight of the total weight of the plurality of granules, such as at least 75, 80, 85, or 90% w/w.
(55) The pharmaceutical composition according to clause (53), wherein the diluent is present in the plurality of granules in an amount of between about 75% w/w and about 98% w/w by weight of the total weight of the plurality of granules, such as between about 85% w/w and about 98% w/w or between about 90% w/w and about 95% w/w.
(56) The pharmaceutical composition according to clauses (52) to (55), wherein the diluent is present in the plurality of granules in an amount of about 93% by weight of the total weight of the plurality of granules.
(57) The pharmaceutical composition according to clauses (38) to (56), wherein the plurality of granules comprise a binder.
(58) The pharmaceutical composition according to clause (57), wherein the binder is present in the plurality of granules in an amount of at least 0.5% w/w by weight of the total weight of the plurality of granules, such as at least 1 or 2% w/w.
(59) The pharmaceutical composition according to clause (57), wherein the binder is present in the plurality of granules in an amount between about 0.5% w/w and about 5% w/w by weight of the total weight of the plurality of granules, such as between about 1% w/w and about 4% w/w or between about 2% w/w and about 3% w/w.
(60) The pharmaceutical composition according to clauses (57) to (59), wherein the binder is present in the plurality of granules in an amount of about 2.5% w/w by weight of the total weight of the plurality of granules.
(61) The pharmaceutical composition according to clauses (38) to (60), wherein the plurality of granules comprise a disintegrant.
(62) The pharmaceutical composition according to clause (61), wherein the disintegrant is present in the plurality of granules in an amount of at least 0.5% w/w by weight of the total weight of the plurality of granules, such as at least 1, 2, or 3% w/w.
(63) The pharmaceutical composition according to clause (61), wherein the disintegrant is present in the plurality of granules in an amount of between about 1% w/w and about 10% w/w by weight of the total weight of the plurality of granules such as between about 2% w/w and about 6% w/w or between about 3% w/w and about 5% w/w.
(64) The pharmaceutical composition according to clauses (61) to (63), wherein the disintegrant is present in the plurality of granules in an amount of about 4% w/w of the total weight of the plurality of granules.
(65) The pharmaceutical composition according to clauses (38) to (64), wherein the plurality of granules comprise a lubricant.
(66) The pharmaceutical composition according to clause (65), wherein the lubricant is present in the plurality of granules in an amount of at least 0.1% w/w by weight of the total weight of the plurality of granules, such as at least 0.5 or 0.8% w/w.
(67) The pharmaceutical composition according to clause (65), wherein the lubricant is present in the plurality of granules in an amount of between about 0.1% w/w and about 2% w/w by weight of the total weight of the plurality of granules, such as between about 0.5% w/w and about 1.5% w/w.
(68) The pharmaceutical composition according to clauses (65) to (67), wherein the lubricant is present in the plurality of granules in an amount of about 1% w/w by weight of the total weight of the plurality of granules.
(69) The pharmaceutical composition according to clauses (53) to (68), wherein the diluent is selected from calcium carbonate, calcium phosphate, dibasic calcium phosphate, tribasic calcium sulfate, calcium carboxymethylcellulose, dextrin derivatives, dextrin, dextrose, fructose, lactitol, lactose (e.g. anhydrous lactose, spray-dried lactose, α-lactose, β-lactose, Tablettose®, various grades of Pharmatose®, Microtose® or Fast-Floc®), lactose monohydratemannitol, methylcellulose polymers such as, e.g., Methocel A®, Methocel A4C®, Methocel A 15C®, Methocel A4M®), hydroxyethylcellulose, hydroxypropylcellulose, L-hydroxypropylycellulose (low substituted), hydroxypropyl methylcellulose (HPMC) (e.g. Methocel E®, F and K, Metolose SH® of Shin-Etsu, grades of Methocel F® and Metolose 65 SH®, the 4,000, 15,000 and 100,000 cps grades of Methocel K®; and the 4,000, 15,000, 39,000 and 100,000 grades of Metolose 90 SH®), sodium carboxymethylcellulose, carboxymethylene, carboxymethylhydroxyethylcellulose and other cellulose derivatives, starches or modified starches (including potato starch, wheat starch, corn starch, rice starch, pregelatinized maize starch), magnesium carbonate, magnesium oxide, maltitol, maltodextrins, maltose, mannitol, kaolin, sorbitol, starch, sucrose, sugar, xylitol, erythritol, isomalt, and mixtures thereof and the like.
(70) The pharmaceutical composition according to clause (69), wherein the diluent is mannitol.
(71) The pharmaceutical composition according to clauses (57) to (70), wherein the binder is selected from hydroxy methyl cellulose, hydroxypropyl cellulose, starch (including corn starch and pregelatinized starch), gelatin, sugars (including sucrose, glucose, dextrose, lactose, and sorbitol), waxes, polyethylene glycol, natural and synthetic gums (e.g. acacia, tragacanth sodium alginate, celluloses, and Veegum), and synthetic polymers such as polymethacrylates and polyvinylpyrrolidone (povidone), ethylcellulose, hydroxyethyl cellulose, polyethylene oxide, carboxymethylcellulose, mannitol, methylcellulose, isomalt, polyvinyl alcohol, and mixtures thereof and the like.
(72) The pharmaceutical composition according to clause (71), wherein the binder is hydroxypropyl methyl cellulose or povidone.
(73) The pharmaceutical composition according to clause (72), wherein the binder is hydroxypropyl methyl cellulose.
(74) The pharmaceutical composition according to clause (73), wherein the binder is 6 cps grade hydroxypropyl methyl cellulose.
(75) The pharmaceutical composition according to clauses (61) to (74), wherein the disintegrant is selected from cross linked polyvinylpyrolidone (crospovidone, polyplyplasdone XL®, kollidon CL®); starches such as maize starch and dried sodium starch glycolate; gums such as maize starch and dried sodium starch glycolate; gums such as alginic acid, sodium alginate, guar gum; croscarmellose sodium; microfine cellulose, low-substituted hydroxypropylcellulose, carboxymethylcellulose, sodium bicarbonate, and soy polysaccharides, and mixtures thereof and the like.
(76) The pharmaceutical composition according to clause (75), wherein the disintegrant is selected from low-substituted hydroxypropyl cellulose, sodium starch glycolate and croscarmellose sodium.
(77) The pharmaceutical composition according to clause (76), wherein the disintegrant is low-substituted hydroxypropyl cellulose.
(78) The pharmaceutical composition according to clauses (65) to (77), wherein the lubricant is selected from sodium oleate, sodium stearate, sodium benzoate, sodium stearate, sodium chloride, stearic acid, sodium stearyl fumarate, calcium stearate, magnesium lauryl sulfate, sodium stearyl fumarate, sucrose esters or fatty acid, zinc, polyethylene glycol, talc, and glyceryl behenate, and mixtures thereof and the like.
(79) The pharmaceutical composition according to clause (78), wherein the lubricant is sodium stearyl fumarate.
(80) The pharmaceutical composition according to clauses (38) to (79) wherein the pharmaceutical composition is an immediate release pharmaceutical composition.
(81) The pharmaceutical composition according to clause (80), wherein at least 75% of the total amount of the N-(2-(tert-butyl)-1-((4,4-difluorocyclohexyl)methyl)-1H-benzimidazol-5-yl)ethanesulfonamide contained in the pharmaceutical composition is released within the first 45 minutes when the pharmaceutical composition is subjected to an in vitro dissolution test employing 0.1 M HCl or pH 6.8 phosphate buffer as the dissolution medium and the dissolution profile is determined as described in the United States Pharmacopoeia at 37° C. using a rotation speed of 50 rpm.
(82) The pharmaceutical composition according to clauses (80) or (81), wherein at least 75% of the total amount of the N-(2-(tert-butyl)-1-((4,4-difluorocyclohexyl)methyl)-1H-benzimidazol-5-yl)ethanesulfonamide contained in the pharmaceutical composition is released within the first 30 minutes when the pharmaceutical composition is subjected to an in vitro dissolution test employing 0.1 M HCl or pH 6.8 phosphate buffer as the dissolution medium and the dissolution profile is determined as described in the United States Pharmacopoeia at 37° C. using a rotation speed of 50 rpm.
(83) The pharmaceutical composition according to clauses (80) to (82), wherein at least 75% of the total amount of the N-(2-(tert-butyl)-1-((4,4-difluorocyclohexyl)methyl)-1H-benzimidazol-5-yl)ethanesulfonamide contained in the pharmaceutical composition is released within the first 15 minutes when the pharmaceutical composition is subjected to an in vitro dissolution test employing 0.1 M HCl or pH 6.8 phosphate buffer as the dissolution medium and the dissolution profile is determined as described in the United States Pharmacopoeia at 37° C. using a rotation speed of 50 rpm.
(84) The pharmaceutical composition according to clauses (38) to (83), wherein when the pharmaceutical composition is subjected to a content uniformity test as described in the European Pharmacopeia the acceptance value is less than 15.
(85) The pharmaceutical composition according to clauses (38) to (84), wherein N-(2-(tert-butyl)-1-((4,4-difluorocyclohexyl)methyl)-1H-benzimidazol-5-yl)ethanesulfonamide, or a pharmaceutically acceptable salt thereof, is stable in the pharmaceutical composition.
(86) The pharmaceutical composition according to clause (85), wherein the active substance is stable during the process to prepare the pharmaceutical composition.
(87) The pharmaceutical composition according to clause (86), wherein the assay of the active substance in the pharmaceutical composition after manufacturing is about 90 to about 110% of nominal, such as about 95 to about 105% or about 98 to about 102%, as determined by a chromatographic method.
(88) The pharmaceutical composition according to clauses (86) or (87), wherein the total amount of degradation products of the active substance in the pharmaceutical composition after manufacturing is less than or equal to about 4 area %, such as less than or equal to 3, 2 or 1 area %, as determined by a chromatographic method.
(89) The pharmaceutical composition according to clauses (85) to (88), wherein the active substance is chemically stable in the pharmaceutical composition for at least about 2 weeks, about 1 month, about 2 months, about 3 months, about 6 months, about 12 months, or about 24 months.
(90) The pharmaceutical composition according to clause (89), wherein the total amount of degradation products of the active substance is less than or equal to about 4 area %, such as less than or equal to 3, 2 or 1 area %, as determined by a chromatographic method when the pharmaceutical composition is stored at 25° C./60% RH and/or 40° C./75% RH for at least about 2 weeks, about 1 month, about 2 months, about 3 months, about 6 months, about 12 months, or about 24 months.
(91) The pharmaceutical composition according to clauses (89) or (90), wherein the assay of the active substance is about 90 to about 110% of nominal, such as about 95 to about 105% or about 98 to about 102%, as determined by a chromatographic method when the pharmaceutical composition is stored at 25° C./60% RH and/or 40° C./75% RH for at least about 2 weeks, about 1 month, about 2 months, about 3 months, about 6 months, about 12 months, or about 24 months.
(92) A process to prepare a plurality of granules according to clause (1) to (37), wherein the process comprises:
   a. mixing a binder and diluent together;
   b. adding a primary granulation fluid to the mixture from step a. and mixing until granules are formed; and
   c. drying the mixture from step b;
   wherein N-(2-(tert-butyl)-1-((4,4-difluorocyclohexyl)methyl)-1H-benzimidazol-5-yl)ethanesulfonamide, or a pharmaceutically acceptable salt thereof, is either mixed with the binder and diluent in step a. or dissolved in the primary granulation fluid.
(93) The process according to clause (92), wherein N-(2-(tert-butyl)-1-((4,4-difluorocyclohexyl)methyl)-1H-benzimidazol-5-yl)ethanesulfonamide, or a pharmaceutically acceptable salt thereof, is mixed with the binder and diluent in step a.
(94) The process according to clauses (92) or (93), wherein step a. further comprises mixing a disintegrant together with the binder and diluent.
(95) The process according to clauses (92) to (94), wherein N-(2-(tert-butyl)-1-((4,4-difluorocyclohexyl)methyl)-1H-benzimidazol-5-yl)ethanesulfonamide, or a pharmaceutically acceptable salt thereof, has a solubility of at least 0.6 mg/mL in the primary granulation fluid at room temperature.
(96) The process according to clauses (92) to (95), wherein the primary granulation fluid is selected from ethanol, 0.1 M HCl, pH 3 citric acid buffer and 5% aqueous SLS.
(97) The process according to clause (96), wherein the primary granulation fluid is 0.1 M HCl.
(98) The process according to clauses (92) to (97), wherein in step b. the primary granulation fluid is added at a rate of about 2 to about 10 mL/min, preferably about 4 to about 7 mL/min, more preferably about 5 to about 6 mL/min.
(99) The process according to clauses (92) to (98), wherein in step b. the mixture is mixed at high speed.
(100) The process according to clauses (92) to (99), wherein in step c. the granules are dried at an elevated temperature.
(101) The process according to clauses (92) to (100), wherein the elevated temperature is greater than 30° C., such as greater than 40° C., greater than 50° C.
(102) The process according to clauses (92) to (101), wherein the process further comprises adding a secondary granulation fluid to step b.
(103) The process according to clause (102), wherein the secondary granulation fluid selected from ethanol, 0.1 M HCl, pH 3 citric acid buffer and 5% aqueous SLS.
(104) The process according to clause (103), wherein the secondary granulation fluid is 0.1 M HCl.
(105) The process according to clauses (102) to (104), wherein the secondary fluid is added over a period of at least 1 minute, such as at least 2 minutes, at least 5 minutes or at least 8 minutes.
(106) The process according to clauses (92) to (105), wherein N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of less than 0.5% w/w by weight of the total weight of the plurality of granules, such as less than 0.4, 0.3, 0.2, 0.15, 0.14, 0.13, 0.12, 0.11, 0.10, 0.08, 0.06, or 0.05% w/w.
(107) The process according to clauses (92) to (106), wherein N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of between about 0.01% w/w and about 0.5% w/w by weight of the total weight of the plurality of granules, such as between about 0.01% w/w and about 0.4% w/w, about 0.01% w/w and about 0.3% w/w, about 0.01% w/w and about 0.2% w/w, about 0.01% w/w and about 0.15% w/w, about 0.01% w/w and about 0.12% w/w, about 0.02% w/w and about 0.12% w/w, or about 0.03% w/w and about 0.10% w/w.
(108) The process according to clauses (92) to (107), wherein N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of about 0.04, 0.10 or 0.12% w/w of the total weight of the plurality of granules.
(109) The process according to clauses (92) to (108), wherein the diluent is present in the plurality of granules in an amount of at least 70% w/w by weight of the total weight of the plurality of granules, such as at least 75, 80, 85, or 90% w/w.
(110) The process according to clauses (92) to (109), wherein the diluent is present in the plurality of granules in an amount of between about 75% w/w and about 98% w/w by weight of the total weight of the plurality of granules, such as between about 85% w/w and about 98% w/w or between about 90% w/w and about 95% w/w.
(111) The process according to clauses (92) to (110), wherein the diluent is present in the plurality of granules in an amount of about 93% by weight of the total weight of the plurality of granules.
(112) The process according to clauses (92) to (111), wherein the binder is present in the plurality of granules in an amount of at least 0.5% w/w by weight of the total weight of the plurality of granules, such as at least 1 or 2% w/w.
(113) The process according to clauses (92) to (112), wherein the binder is present in the plurality of granules in an amount between about 0.5% w/w and about 5% w/w by weight of the total weight of the plurality of granules, such as between about 1% w/w and about 4% w/w or between about 2% w/w and about 3% w/w.
(114) The process according to clauses (92) to (113), wherein the binder is present in the plurality of granules in an amount of about 2.5% w/w by weight of the total weight of the plurality of granules.
(115) The process according to clauses (94) to (114), wherein the disintegrant is present in the plurality of granules in an amount of at least 0.5% w/w by weight of the total weight of the plurality of granules, such as at least 1, 2, or 3% w/w.
(116) The process according to clauses (94) to (115), wherein the disintegrant is present in the plurality of granules in an amount of between about 1% w/w and about 10% w/w by weight of the total weight of the plurality of granules, such as between about 2% w/w and about 6% w/w or between about 3% w/w and about 5% w/w.
(117) The process according to clauses (94) to (116), wherein the disintegrant is present in the plurality of granules in an amount of about 4% w/w of the total weight of the plurality of granules.
(118) The process according to clauses (92) to (117), wherein the diluent is selected from calcium carbonate, calcium phosphate, dibasic calcium phosphate, tribasic calcium sulfate, calcium carboxymethylcellulose, dextrin derivatives, dextrin, dextrose, fructose, lactitol, lactose (e.g. anhydrous lactose, spray-dried lactose, α-lactose, β-lactose, Tablettose®, various grades of Pharmatose®, Microtose® or Fast-Floc®), lactose monohydratemannitol, methylcellulose polymers such as, e.g., Methocel A®, Methocel A4C®, Methocel A 15C®, Methocel A4M®), hydroxyethylcellulose, hydroxypropylcellulose, L-hydroxypropylycellulose (low substituted), hydroxypropyl methylcellulose (HPMC) (e.g. Methocel E®, F and K, Metolose SH® of Shin-Etsu, grades of Methocel F® and Metolose 65 SH®, the 4,000, 15,000 and 100,000 cps grades of Methocel K®; and the 4,000, 15,000, 39,000 and 100,000 grades of Metolose 90 SH®), sodium carboxymethylcellulose, carboxymethylene, carboxymethylhydroxyethylcellulose and other cellulose derivatives, starches or modified starches (including potato starch, wheat starch, corn starch, rice starch, pregelatinized maize starch), magnesium carbonate, magnesium oxide, maltitol, maltodextrins, maltose, mannitol, kaolin, sorbitol, starch, sucrose, sugar, xylitol, erythritol, and isomalt, and mixtures thereof and the like.
(119) The process according to clause (118), wherein the diluent is mannitol.
(120) The process according to clauses (92) to (119), wherein the binder is selected from hydroxy methyl cellulose, hydroxypropyl cellulose, starch (including corn starch and pregelatinized starch), gelatin, sugars (including sucrose, glucose, dextrose, lactose, and sorbitol), waxes, polyethylene glycol, natural and synthetic gums (e.g. acacia, tragacanth sodium alginate, celluloses, and Veegum), and synthetic polymers such as polymethacrylates and polyvinylpyrrolidone (povidone), ethylcellulose, hydroxyethyl cellulose, polyethylene oxide, carboxymethylcellulose, mannitol, methylcellulose, isomalt, polyvinyl alcohol, and mixtures thereof and the like.
(121) The process according to clause (120), wherein the binder is hydroxypropyl methyl cellulose or povidone.
(122) The process according to clause (121), wherein the binder is hydroxypropyl methyl cellulose.
(123) The process according to clause (122), wherein the binder is 6 cps grade hydroxypropyl methyl cellulose.
(124) The process according to clauses (94) to (123), wherein the disintegrant is selected from cross linked polyvinylpyrolidone (crospovidone, polyplyplasdone XL®, kollidon CL®); starches such as maize starch and dried sodium starch glycolate; gums such as maize starch and dried sodium starch glycolate; gums such as alginic acid, sodium alginate, guar gum; croscarmellose sodium; microfine cellulose, low-substituted hydroxypropylcellulose, carboxymethylcellulose, sodium bicarbonate, and soy polysaccharides, and mixtures thereof and the like.
(125) The process according to clause (124), wherein the disintegrant is selected from low-substituted hydroxypropyl cellulose, sodium starch glycolate and croscarmellose sodium.
(126) The process according to clause (125), wherein the disintegrant is low-substituted hydroxypropyl cellulose.
(127) The process according to clauses (92) to (126), wherein the bulk density of the plurality of the granules prepared is between about 0.1 and about 0.7 g/mL, such as between about 0.2 and about 0.6 g/mL, or between about 0.3 and about 0.5 g/mL.
(128) The process according to clauses (92) to (127) further comprising adding a lubricant to step b.
(129) The process according to clause (128), wherein the lubricant is selected from sodium oleate, sodium stearate, sodium benzoate, sodium stearate, sodium chloride, stearic acid, sodium stearyl fumarate, calcium stearate, magnesium lauryl sulfate, sodium stearyl fumarate, sucrose esters or fatty acid, zinc, polyethylene glycol, talc, and glyceryl behenate, and mixtures thereof and the like.
(130) The process according to clause (130), wherein the lubricant is sodium stearyl fumarate.
(131) The pharmaceutical composition according to clauses (38) to (91) for use as a medicament.
(132) The pharmaceutical composition according to clauses (38) to (91) for use in a disease state or disorder in which dysfunction of CB₁ and/or CB₂ receptors are present or implicated.
(133) The pharmaceutical composition according to clauses (38) to (91) for use in the treatment of an anorexia associated condition; a cachexia associated condition; or anorexia nervosa

### EXAMPLES

The following abbreviations have been used in the Examples:
API active pharmaceutical ingredient, which in the present case is N-(2-(tert-butyl)-1-((4,4-difluorocyclohexyl)methyl)-1H-benzimidazol-5-yl)ethanesulfonamide (Compound I)
AV acceptance value
HPMC hydroxypropyl methylcellulose
NMT not more than

### Example 1—Forced Degradation Study of N-(2-(tert-butyl)-1-((4,4-difluorocyclohexyl)methyl)-1H-benzimidazol-5-yl)ethanesulfonamide

A forced degradation of N-(2-(tert-butyl)-1-((4,4-difluorocyclohexyl)methyl)-1H-benzimidazol-5-yl)ethanesulfonamide study (active substance) was performed under a number of stressed conditions for up to 13 days. The active substance was shown to be stable to degradation as a solution at room temperature, at 100° C. as a solid, under 1M acidic conditions at room temperature and at 60° C., under 0.5M basic conditions at room temperature and at 60° C., as a solution heated to 60° C., and under photostability conditions as a solid. The active substance showed slight degradation under peroxide conditions, under 1M acidic conditions and 0.5M basic conditions when these tests were extended to 80° C., and as a solution when heated to 80° C. Major degradation of the active substance as a solution under photostability conditions occurred.

### Materials

| Active substance | N-(2-(tert-butyl)-1-((4,4-difluorocyclohexyl)methyl)-1H- | |
|---|---|---|
| | benzimidazol-5-yl)ethanesulfonamide | |
| | ex- Onyx Scientific Ltd | Batch no: CB988E |
| LCMS Water | ex- Onyx Scientific Ltd | N/A |
| Acetonitrile | ex- Sigma-Aldrich | Lot no: - STBJ1963 |
| Ammonium acetate | ex- Fluka | Lot no: - H2010 |
| 6.0M Hydrochloric Acid | ex- Fluka | Lot no: - H1370 |
| 1M Sodium Hydroxide | ex- Fisher | Lot no: - 1871868 |
| 30% Hydrogen peroxide | ex- Sigma-Aldrich | Lot no: - MKBS2987V |

Analysis
HPLC Conditions

| | |
|---|---|
| Instrument: | Agilent 1100/1200 |
| Column: | YMC Triart Phenyl, 4.6 × 150 mm. 3 μm particle size |
| | (ex-YMC P.N.TPH12S03-1546PTH) |
| Mobile Phase: | A - 10 mM Ammonium acetate pH 6.0 |
| | B - Acetonitrile |
| Flow Rate: | 1.0 ml · min − 1 |
| Injection Volume: | 5 μl |
| Detection: | UV @ 220 nm |
| Column Temp: | 30° C. |
| Post Run: | 4 minutes |

| Gradient: | Time (mins) | % A | % B |
|---|---|---|---|
| | 0 | 80 | 20 |
| | 20 | 35 | 65 |
| | 26 | 5 | 95 |
| | 30.5 | 5 | 95 |
| | 31 | 80 | 20 |

| | |
|---|---|
| Diluent: | Acetonitrile:deionised water (1:1) |
| Standard Solution: | Make up a solution of 0.2 mg/ml. This is to be made up fresh |
| | prior to use at T = 3 and T = 7 days. |

### Stock Solution Preparation

Accurately weigh 80 mg of the active substance into a 100 mL volumetric flask and dissolve in 50 mL of diluent. Dilute the resulting solution to volume with diluent and mix well.

### Procedure

Table 1 below details the different study tests performed. All solution samples are prepared and stored in sealed vials and heated samples are cooled to ambient prior to sampling to prevent evaporation. Analysis took place on days 3 and 7 after stressing initiation for solutions described for Tests 1-6 and once exposure was complete for samples described in Tests 7-10. Due to the low levels of degradation observed at 7 days, Tests 3, 4 and 5 were heated to 80° C. for a further 4 days and analysed, and Test 6 was analysed after a further 6 days.

The photostability samples were exposed to a total of 1.92 million lux hours and an integrated near-UV energy of 374.6 Whm⁻².

TABLE 1
forced degradation study tests

| | | Sample preparation for analysis |
|---|---|---|
| Test | Description | after study |
| Test 1 - | Accurately pipette 5 ml of stock | Pipette 1 ml of Control Solution into |
| Control | solution and 5 ml of diluent into a | a 2 ml volumetric flask, make up to |
| | suitable container and leave at | volume with diluent and mix well. |
| | ambient temperature. | |
| Test 2 - Solid | Store 150 mg of sample at 100° C. | Dissolve in diluent to a |
| state | | concentration of 0.2 mg · ml⁻¹_{.} |
| Test 3 - Acid | Accurately pipette 5 ml of Stock | Pipette 1 ml of Acid Sample Solution |
| (1M) | Solution and 5 ml of 2M HCl solution | into a 2 ml volumetric flask, make up |
| | into a suitable container and leave | to volume with diluent and mix well. |
| | at ambient temperature. | |
| Test 3- Acid | Accurately pipette 5 ml of diluent | Pipette 1 ml of Acid Blank Solution |
| Blank | and 5 ml of 2M HCl solution into a | into a 2 ml volumetric flask, make up |
| (1M) | suitable container and leave at | to volume with diluent and mix well. |
| | ambient temperature. | |
| Test 3 - Acid | Accurately pipette 5 ml of Stock | Pipette 1 ml of Acid Sample Solution |
| (1M heated) | Solution and 5 ml of 2M HCl solution | into a 2 ml volumetric flask, make up |
| | into a suitable container and heat at | to volume with diluent and mix well. |
| | 60° C. | |
| Test 3 -Acid | Accurately pipette 5 ml of diluent | Pipette 1 ml of Acid Blank Solution |
| Blank | and 5 ml of 2M HCl solution into a | into a 2 ml volumetric flask, make up |
| (1M heated) | suitable container and heat at 60° C. | to volume with diluent and mix well. |
| Test 4 - Base | Accurately pipette 5 ml of Stock | Pipette 1 ml of Base Sample |
| (0.5M) | Solution and 5 ml of 1M NaOH | Solution into a 2 ml volumetric flask, |
| | solution into a suitable container | make up to volume with diluent and |
| | and leave at ambient temperature. | mix well. |
| Test 4 - Base | Accurately pipette 5 ml of diluent | Blank - Pipette 1 ml of Base Blank |
| Blank (0.5M) | and 5 ml of 1M NaOH solution into a | Solution into a 2 ml volumetric flask, |
| | suitable container and leave at | make up to volume with diluent and |
| | ambient temperature. | mix well. |
| Test 4-Base | Accurately pipette 5 ml of Stock | Pipette 1 ml of Base Sample |
| (0.5M heated) | Solution and 5 ml of 1M NaOH | Solution into a 2 ml volumetric flask, |
| | solution into a suitable container | make up to volume with diluent and |
| | and heat at 60° C. | mix well. |
| Test 4 - Base | Accurately pipette 5 ml of diluent | Blank - Pipette 1 ml of Base Blank |
| Blank | and 5 ml of 1M NaOH solution into a | Solution into a 2 ml volumetric flask, |
| (0.5M heated) | suitable container and heat at 60° C. | make up to volume with diluent and |
| | | mix well. |
| Test 5 - Heat | Accurately pipette 5 ml of Stock | Pipette 1.0 ml of heated sample |
| (60° C.) | Solution and 5 ml of diluent into a | solution into a 2 ml volumetric flask, |
| | suitable container and heat at 60° C. | make up to volume with diluent and |
| | | mix well. |
| Test 6 - | Accurately pipette 5 ml of Stock | Pipette 1.0 ml of Peroxide Sample |
| Peroxide | Solution and 5 ml of 3% Hydrogen | solution into a 2 ml volumetric flask, |
| (1.5%) | peroxide solution into a suitable | make up to volume with diluent and |
| | container and leave at ambient | mix well. |
| | temperature. | |
| Test 6 - | Accurately pipette 5 ml of diluent | Pipette 1 ml of Peroxide Blank |
| Peroxide | and 5 ml of 3% Hydrogen peroxide | Solution into a 2 ml volumetric flask, |
| Blank (1.5%) | solution into a suitable container | make up to volume with diluent and |
| | and leave at ambient temperature. | mix well. |
| Test 7 - | Accurately pipette 5 ml of Stock | Once exposure is complete, pipette |
| Photostability | Solution and 5 ml of diluent into a | 1.0 ml of sample solution into a 2 ml |
| (Solution | suitable container, wrapped in | volumetric flask, make up to volume |
| Control) | aluminium foil. Expose to an overall | with diluent and mix well. |
| | illumination of no less than 1.2 | |
| | million lux hours and integrated | |
| | near-UV energy of no less than 200 | |
| | watt hours per square metre. | |
| Test 8 - | Accurately pipette 5 ml of Stock | Once exposure is complete, pipette |
| Photostability | Solution and 5 ml of diluent into a | 1.0 ml of sample solution into a 2 ml |
| (Solution | suitable container. Expose to an | volumetric flask, make up to volume |
| Treated) | overall illumination of no less than | with diluent and mix well. |
| | 1.2 million lux hours and integrated | |
| | near-UV energy of no less than 200 | |
| | watt hours per square metre. | |
| Test 9 - | Place a thin layer of the solid | Once exposure is complete, the |
| Photostability | sample in a glass dish and wrap in | sample is to be dissolved in diluent |
| (Solid Control) | aluminium foil. Expose to an overall | to a concentration of 0.2 mg · ml⁻¹. |
| | illumination of no less than 1.2 | |
| | million lux hours and an integrated | |
| | near-UV energy of no less than 200 | |
| | watt hours per square metre. | |
| Test 10 - | Place a thin layer of the solid | Once exposure is complete, the |
| Photostability | sample in a glass dish. Expose to an | sample is to be dissolved in diluent |
| (Solid Treated) | overall illumination of no less than | to a concentration of 0.2 mg · ml⁻¹. |
| | 1.2 million lux hours and an | |
| | integrated near-UV energy of no | |
| | less than 200 watt hours per square | |
| | metre. | |

### Results

### Observations

TABLE 2
Forced degradation - observations

| Test | Condition | Sample observations |
|---|---|---|
| 1 | Control | Clear colourless solution. No change over study period. |
| 2 | Solid state | Off-white solid turning to very faint orange solid over study |
| | 100° C. | period. |
| 3 | Acid | Clear colourless solution. No change over study period. |
| 3 | Acid 60° C. | Clear colourless solution. No change over study period. |
| 4 | Base | Clear colourless solution. No change over study period. |
| 4 | Base 40° C. | Clear colourless solution. No change over study period. |
| 5 | Heat 60° C. | Clear colourless solution. No change over study period |
| 6 | Peroxide | Clear colourless solution. No change over study period |
| 7 | Photostability | Clear colourless solution. No change over study period. |
| | (Solution | |
| | Control) | |
| 8 | Photostability | Clear colourless solution turning to a clear yellow solution |
| | (Solution | over study period. |
| | Treated) | |
| 9 | Photostability | Off white solid. No change over study period. |
| | (Solid | |
| | Control) | |
| 10 | Photostability | Off-white solid turning to very faint orange solid over study |
| | (Solid | period. |
| | Treated) | |

### Analysis

TABLE 3
forced degradation results for Tests 1-6

| | Assay of active substance (area %) | | | | |
|---|---|---|---|---|---|
| | Timepoint | | | | |
| | 0 | 3 | 7 | 11 | 13 |
| Test | days | days | days | days | days |
| 1 - solution control | 99.40 | 99.42 | 99.30 | 99.39 | |
| 2 - solid state at 100° C. | | 99.43 | 99.40 | | |
| 3 - acid (1M) ambient | | 99.40 | 99.41 | | |
| temperature | | | | | |
| 3 - acid (1M) at 60° C. until | | 99.37 | 99.35 | 98.87 | |
| day 7, 80° C. until day 11 | | | | | |
| 4 - base (0.5M) ambient | | 99.40 | 99.37 | | |
| temperature | | | | | |
| 4 - base (0.5M) at 60° C. | | 99.34 | 99.32 | 99.16 | |
| until day 7, 80° C. until day | | | | | |
| 11 | | | | | |
| 5 - solution at 60° C. until | | 99.13 | 99.34 | 98.00 | |
| day 7, 80° C. until day 11 | | | | | |
| 6 - peroxide (1.5%) | | 98.50 | 97.75 | | 96.58 |

TABLE 4
forced degradation results for Tests 7-10

| | Assay of active |
|---|---|
| Test | substance (area %) |
| 7 - photocontrol solution | 99.40 |
| 8 - photo treated solution | 74.53 |
| 9 - photocontrol solid | 99.40 |
| 10 - phototreated solid | 99.31 |

### Example 2—Preformulation Studies—Compatibility of Active Ingredient with Capsule Shells and Wet Granulation Excipients

A preformulation study was performed in order to select excipients for the development of an immediate release capsule containing a low dose of granulated N-(2-(tert-butyl)-1-((4,4-difluorocyclohexyl)methyl)-1H-benzimidazol-5-yl)ethanesulfonamide. The chemical stability of granulated N-(2-(tert-butyl)-1-((4,4-difluorocyclohexyl)methyl)-1H-benzimidazol-5-yl)ethanesulfonamide was evaluated for up to 28 days at 40° C. and 50° C. Both types of capsule shells evaluated (HPMC and PEG-softened hard gelatin shells) were compatible with the active ingredient. Of the six granulation fluids studied, four showed sufficient solubility to allow the active ingredient to be solution doped on a granule and filed into a capsule for the potential high dose of 200 μg. None of these granulation systems showed any signs of degradation for potential overnight drying under ambient conditions. However, only two of these granulation fluids (0.1 M HCl and ethanol) gave enough solubility to fill size 2 to 4 capsules.

The 28 day stability of three prototype wet granulation formulations showed no evidence of active ingredient degradation. The remaining formulation gave unacceptably high peak areas of one compound-related substance after 28 days at 50° C. From this, two excipients can be ruled out from further consideration because of their chemical incompatibility with the active ingredient (magnesium stearate and microcrystalline cellulose). However, there remain several candidate excipients for each functional type that appear to be active ingredient-compatible (binders, disintegrants, diluents and lubricants).

### Materials

TABLE 5
Excipients used in pre-formulation study

| | | Target quantity of | |
|---|---|---|---|
| Excipient | Function | excipient per vial | |
| Mannitol (Pearlitol SD100) | Diluent for wet | 280-320 | mg |
| Microcrystalline cellulose | granulation/(dry | | |
| (Avicel PH101) | excipient) | | |
| Pruv grade of sodium | Lubricant for | 25-30 | mg |
| stearyl fumarate | encapsulation (dry | | |
| Magnesium Stearate | excipient) | | |
| (Ligamed) | | | |
| HPMC (Methocel E6 | Binder for wet | | |
| Premium i.e. 6 cps grade) | granulation | | |
| Kollidon K25 (Povidone | | | |
| K25, PVP) | | | |
| Hydroxypropyl cellulose | Disintegrant | | |
| LS (L-HPC-B1) | | | |
| Sodium starch glycolate | | | |
| (Explotab) | | | |
| Croscarmellose sodium | | | |
| (AcDiSol) | | | |
| HMPC capsule | Capsule shell | 1 | Capsule |
| (White Capsugel VCaps | (dry excipient) | | |
| Plus size 4) | | | |
| Hard Gelatin capsule | | | |
| (Green/grey Qualicaps | | | |
| Quali-G PEG size 4) | | | |
| Ethanol absolute | Volatile liquid solvent | 100 | μL |

| | |
|---|---|
| 50:50 EtOH:water (ethanol | for wet granulation |
| absolute:deionised water) | |
| 0.1M HCl | |
| pH 3 citric acid buffer | |
| (0.1M citric acid + 0.2M | |
| Na₂HPO₄) | |
| 5% SLS (sodium lauryl | |
| sulphate) aq. | |
| 5% Tween 80 (polysorbate | |
| 80) aq. | |
| (N-(2-(tert-butyl)-1-((4,4- | Active Ingredient |
| difluorocyclohexyl)methyl)- | |
| 1H-benzimidazol-5- | |
| yl)ethanesulfonamide) | |
| (ex IPCA; Supplier Lot ID | |
| JCM1019B) | |

### Analytical Methodology

UPLC Conditions

| Column: | Waters Acquity BEH Shield | | |
|---|---|---|---|
| | 100 × 2.1 mm, 1.7 μm | | |
| Column Temp. | 40° C. | | |
| Sample manager wash (UPLC) | Acetonitrile | | |
| Sample manager purge (UPLC) | HPLC Water: ACN 50:50% v/v | | |
| Mobile Phase A (for volatile solvents) | 0.18 mM ammonia solution | | |
| Mobile Phase A (for capsule shells) | 25% ammonia solution | | |
| Mobile Phase A (for prototype granules) | 10 mM ammonium acetate pH 7.0 | | |
| Mobile Phase B | Acetonitrile | | |
| Flow Rate | 0.3 mL/min | | |
| Injection volume | 10 μL (for 0.01 mg/mL nominal concentration) | | |
| | 5 μL (for 0.02 mg/mL nominal concentration) | | |
| Wavelength | 215 nm | | |
| | Time | MP* A | MP* B |
| | (min) | (%) | (%) |
| Gradient program | 0 | 95 | 5 |
| | 10 | 5 | 95 |
| | 10.1 | 95 | 5 |
| | 15 | 95 | 5 |
| | 15.1 | | |
| | 20 | | |

| | |
|---|---|
| Diluent | All analytical solutions were prepared to a |
| | nominal concentration of 0.001 mg/mL in 50:50 |
| | acetonitrile:water |

*MP = mobile Phase

### Stability Studies

Note the incubation for the stability studies were all performed in a similar way:
All were put into 4 mL amber glass vials.
The Initial samples were not analysed as soon as sampled but instead were frozen at −20° C. until the point of analysing the incubated samples (e.g after 28 days at 25, 40 or 50° C.) to avoid degradation of the active ingredient.

### Part 1—Solubility and Compatibility of Active Ingredient in Volatile Solvents

The active ingredient was dissolved in different solvents and the stability of the solution was assessed after being held overnight at ambient temperature. The following solvents were studied: ethanol, 50:50 EtOH:Water, 0.1 M HCl, pH 3 citric acid buffer, 5% SLS aq. and 5% Tween 80 aq.

### Methodology

For each solvent above:
15.5-16.5 mg active ingredient was dispensed into vials
Solvent aliquots were added into the vial as per Table 6: Aliquot additions required of the volatile solvents Table 6 and mixing performed using a magnetic flea at 250 RPM for between 30 and 180 minutes to be certain no more active compound would dissolve.
Further solvent aliquots were added (to a maximum of 30 mL total solvent), only if the active ingredient had failed to completely dissolve after the previous aliquot.
The stability of the active ingredient was assessed after incubation at 20-25 C.° for 20-28 hours in the solvent: the HPLC data (assay and related substances) was compared to the sample taken at initial (and frozen at −20° C. to prevent degradation).

TABLE 6
Aliquot additions of the volatile solvents

| Amount of active ingredient per | 15.5-16.5 | | |
|---|---|---|---|
| bottle (mg) | | | |
| Target solubility (mg/mL) | 7 | 3 | 0.6 |
| Solvent Aliquot # | 1 | 2 | 3 |
| Aliquot of solvent to add (mL) | 2.3 | 3.0 | 21.3 |
| Total volume of Solvent in | 2.3 | 5.3 | 26.6 |
| bottle (mL) | | | |

### Results

This study involved assessing the solubility and the chemical stability of the active ingredient in different volatile solvents for use in wet granulation. The solubility was measured visually after mixing (by the disappearance of undissolved solids). Increasing quantities of each solvent were added as detailed above. The results are summarised in Table 7:
The solubility of the active ingredient was found to be highest in both ethanol and 0.1 M HCl: 7 mg/ml, estimated to be sufficient to achieve the highest potential dose (200 μg) in the smallest capsule size (Size 4).
The next highest solubilities were achieved in aqueous 5% SIS and pH 3 Citric buffer. These solvents showed a lower solubility: >0.6 mg/mL, estimated to be sufficient to achieve the highest potential dose (200 μg) in the largest capsule size (Size 00).
The active ingredient didn't dissolve in 50:50 EtOH:water or 5% Tween 80 even at the maximum dilution (<0.6 mg/mL) i.e. insufficient for dosing 200 μg in any capsule size. Therefore, both 50:50 EtOH:water and 5% Tween 80 were eliminated from further studies.

TABLE 7
Observations of active ingredient solubilization in volatile
solvents as a function of active ingredient concentration

| | +Solvent Aliquot 1 | +Solvent Aliquot 2 | +Solvent Aliquot 3 | |
|---|---|---|---|---|
| | Active ingredient | Active ingredient | Active ingredient | |
| | concentration: | concentration: | concentration: | |
| | 7 mg/mL = | 3 mg/mL = | 0.6 mg/mL = | |
| | Capsule Size 2 | Capsule Size 4 | Capsule Size 00 | |
| Solvent | Time to dissolve (note DND = did not dissolve) | | | |
| Ethanol absolute | 2 min, 44 secs | N/A | N/A | |
| 50:50 EtOH:water | DND | DND | DND | |
| 0.1M HCl | 4 min, 8 secs | N/A | N/A | |
| pH 3 citric acid buffer | DND | DND | 180 | min |
| 5% SLS aq. | DND | DND | 70 | min |

| | | | |
|---|---|---|---|
| 5% Tween 80 aq. | DND | DND | DND |

Chemical stability was determined by assay (results in Table 8) and quantification of potential active ingredient related substances (shown in

Table 9). These were identified by overlays of the chromatograms of the active ingredient solutions, compared at initial and after a 20-28 hour hold at ambient temperature (and also with the control samples without the active ingredient). The objective was to establish whether any active ingredient-related substances might be increasing as a result of the ambient hold. Overall, there is no evidence for any active ingredient degradation:
The total impurities didn't exceed 1.17% of the total peak area in any case. Most of these peaks also appear in the active ingredient standard (as noted in the asterisk in Table 9), so are likely contaminants or pre-existing impurities in the as-delivered active ingredient.
No peaks grew between the initial sample (frozen upon sampling) and the sample held at 20-28 hours at ambient temperature.

Every impurity peak is listed in Table 9. Peaks present in placebo/diluent but bigger in samples are highlighted in bold. Those that are associated with the active ingredient standard are marked by *. However it is not possible to say whether all of the other peaks are truly active ingredient related because multiple mobile phase related peaks are seen with inconsistent elution times. In particular those between RRT 0.38 and 0.86 are not consistent between replicates.

TABLE 8
Assay results for the active ingredient dissolved in volatile
solvents initially vs. after 24 hours at ambient temperature

| Formulation | | | Assay (as % |
|---|---|---|---|
| description | Stability | Vial replicate | of nominal) |
| Ethanol | Initial | 1 | 90.6 |
| Absolute | | 2 | 107.1 |
| | | Mean | 98.8 |
| | 24 hours | 1 | 100.2 |
| | | 2 | 97.7 |
| | | Mean | 99.0 |
| 0.1M HCl | Initial | 1 | 100.6 |
| | | 2 | 127.9 |
| | | Mean | 114.4 |
| | 24 hours | 1 | 107.7 |
| | | 2 | 105.4 |
| | | Mean | 106.7 |
| pH 3 citric | Initial | 1 | 108.8 |
| acid | | 2 | 110.4 |
| buffer | | Mean | 109.6 |
| | 24 hours | 1 | 109.6 |
| | | 2 | 82.1 |
| | | Mean | 95.8 |
| 5% SLS aq. | Initial | 1 | 105.0 |
| | | 2 | 101.7 |
| | | Mean | 103.3 |
| | 24 hours | 1 | 99.4 |
| | | 2 | 102.3 |
| | | Mean | 100.8 |

TABLE 9
Peak Area results (as % of Total) for impurities in volatile solvent samples

| | | Ethanol absolute | | | | 0.1 M HCl | | | | pH 3 citric acid buffer | | | | 5% SLS aq. | | | |
|---|---|---|---|---|---|---|---|---|---|---|---|---|---|---|---|---|---|
| | | Initial | | 24 hour | | Initial | | 24 hour | | Initial | | 24 hour | | Initial | | 24 hour | |
| Samples | | 1 | 2 | 1 | 2 | 1 | 2 | 1 | 2 | 1 | 2 | 1 | 2 | 1 | 2 | 1 | 2 |
| RRT | 0.38 | ND | 0.07 | ND | ND | 0.10 | 0.08 | ND | 0.07 | ND | 0.27 | 0.26 | 0.14 | ND | ND | 0.10 | 0.07 |
| +0.02 | 0.41 | ND | ND | ND | ND | <0.05 | <0.05 | ND | ND | ND | 0.26 | 0.28 | 0.13 | ND | ND | <0.05 | <0.05 |
| | 0.61 | ND | 0.08 | ND | ND | 0.13 | 0.08 | <0.05 | <0.05 | ND | 0.05 | 0.07 | 0.10 | ND | ND | 0.07 | 0.06 |
| | 0.62 | ND | 0.09 | ND | ND | 0.15 | 0.09 | <0.05 | 0.05 | ND | 0.06 | 0.07 | 0.11 | ND | ND | 0.08 | 0.08 |
| | 0.83 | ND | 0.18 | ND | ND | 0.27 | <0.05 | ND | 0.11 | ND | 0.21 | 0.30 | 0.16 | ND | ND | 0.30 | 0.16 |
| | 0.84* | 0.37 | 0.33 | 0.33 | 0.35 | 0.31 | 0.26 | 0.27 | 0.34 | 028 | 0.30 | 0.30 | 0.37 | 0.32 | 0.32 | 0.32 | 0.30 |
| | 0.85 | ND | ND | ND | ND | <0.05 | 0.08 | ND | 0.07 | ND | ND | ND | ND | ND | ND | ND | NBD |
| | 0.86^{$} | <0.05 | 0.16 | <0.05 | <0.05 | 0.20 | 0.05 | <0.05 | 0.05 | <0.05 | <0.05 | <0.05 | 0.15 | <0.05 | <0.05 | 0.05 | 0.07 |
| | 0.92 | <0.05 | <0.05 | <0.05 | <0.05 | 0.18 | 0.14 | 0.09 | 0.09 | 0.10 | 0.09 | 0.06 | 0.06 | 0.05 | <0.05 | 0.05 | 0.05 |
| | 0.98* | 0.20 | 0.22 | 0.22 | 0.20 | 0.22 | 0.22 | 0.24 | 0.22 | 0.23 | 0.21 | 0.22 | 0.21 | 0.24 | 0.24 | 0.24 | 0.24 |
| | 0.98* | 0.12 | 0.12 | 0.13 | 0.14 | 0.12 | 0.12 | 0.12 | 0.13 | 0.11 | 0.13 | 0.13 | 0.14 | 0.12 | 0.12 | 0.13 | 0.12 |
| | 1.04* | 0.05 | <0.05 | <0.05 | <0.05 | <0.05 | <0.05 | 0.05 | <0.05 | 0.09 | 0.09 | 0.09 | 0.09 | 0.05 | <0.05 | 0.06 | 0.06 |
| | 1.23* | 0.08 | 0.07 | 0.07 | 0.07 | 0.22 | 0.23 | 0.16 | 0.15 | 0.16 | 0.12 | 0.10 | 0.10 | 0.06 | 0.07 | 0.06 | 0.07 |
| | 1.24* | <0.05 | <0.05 | <0.05 | <0.05 | <0.05 | <0.05 | <0.05 | 0.05 | <0.05 | <0.05 | <0.05 | 0.06 | <0.05 | <0.05 | <0.05 | <0.05 |
| | 1.35* | 0.08 | 0.09 | 0.08 | 0.08 | 0.05 | 0.06 | 0.07 | 0.06 | ND | ND | ND | ND | 0.07 | 0.08 | 0.07 | 0.06 |
| | 1.36* | 0.06 | 0.06 | 0.06 | 0.06 | 0.05 | 0.06 | <0.05 | 0.05 | ND | ND | ND | ND | 0.05 | 0.05 | <0.05 | <0.05 |

| | | | | | | | | | | | | | | | | |
|---|---|---|---|---|---|---|---|---|---|---|---|---|---|---|---|---|
| Total | 0.96 | 1.47 | 0.89 | 0.90 | 2.00 | 1.47 | 1.00 | 1.44 | 0.97 | 1.79 | 1.88 | 1.82 | 0.96 | 0.88 | 1.53 | 1.34 |
| Total | 0.96 | 0.89 | 0.89 | 0.90 | 1.15 | 1.17 | 1.00 | 1.16 | 0.97 | 0.94 | 0.90 | 1.03 | 0.96 | 0.88 | 0.93 | 0.90 |
| excluding | | | | | | | | | | | | | | | | |
| artifact | | | | | | | | | | | | | | | | |

*These peaks are also observed in the active ingredient only analytical dilution (standard) at a comparable area %.
^{$}RRT 0.86 peak PDA spectra is completely unrelated to the active ingredient

Part 2—Compatibility of Active Ingredient with HPMC and Gelatin Capsule Shells

Binary mixtures of the active ingredient with the capsules shells were made as follows:
An active ingredient doping solution (active ingredient dissolved in ethanol) was made (3 mg/ml). A 100 μL aliquot of this doping was used to ‘spike’ the 10 vials of each capsule shell type as described in Table 5. The nominal quantity of active ingredient per vial was therefore 50 μg.
No doping solution was added to the three control vials of the excipient.
The active vials were left open overnight to evaporate all the ethanol, before closing the vials.
Both the active and placebo control vials were placed on stability for up to 28 days at 25° C. and 50° C.

### Results

Table 10 shows the assay results of the active ingredient in HPMC and gelatin capsules.

TABLE 10
Assay results of active ingredient in HPMC and gelatin capsules

| Incubation | | | HPMC | Gelatin |
|---|---|---|---|---|
| temperature | days | vial | Assay (%) | Assay (%) |
| n/a | initial | 1 | 96.5 | 99.0 |
| | | 2 | 99.0 | 100.2 |
| | | mean | 97.8 | 99.6 |
| 25° C. | 14 | 3 | N/A* | 97.4 |
| | | 4 | 98.9 | 98.9 |
| | | mean | 95.8 | 98.1 |
| | 28 | 5 | 99.3 | 98.8 |
| | | 6 | 99.0 | 99.4 |
| | | mean | 99.1 | 99.1 |
| 50° C. | 14 | 7 | 98.9 | 99.3 |
| | | 8 | 98.7 | 99.4 |
| | | mean | 98.8 | 99.3 |
| | 28 | 9 | 98.8 | 99.4 |
| | | 10 | 98.9 | N/A* |
| | | mean | 98.9 | 89.3 |

*Samples were spilt during preparation and so results are discounted.

Overall, the % assay for both sample sets appear consistent across all conditions and timepoints, indicating there is no degradation of the active ingredient for either HPMC or gelatin capsules.

The peak areas of potential active ingredient related substances are shown in Table 11. There is no evidence of active ingredient degradation in both capsule types since the total impurities remained below 1% with no increase with incubation time or temperature for any impurity.

TABLE 11
Summary of related substances in HPMC and gelatin capsules

| | | | | RRT | | | | | | | |
|---|---|---|---|---|---|---|---|---|---|---|---|
| Sample | Condition | Days | Vial | 0.92 | 0.97 | 0.98 | 1.04 | 1.10 | 1.35 | 1.36 | total |
| HPMC | n/a | initial | 1 | 0.08 | ND | 0.3 | 0.07 | 0.09 | 0.13 | 0.13 | 0.81 |
| | | | 2 | 0.07 | ND | 0.31 | 0.07 | 0.10 | 0.13 | 0.13 | 0.82 |
| | 25° C. | 14 | 3 | 0.08 | ND | 0.31 | 0.08 | 0.09 | 0.13 | 0.13 | 0.82 |
| | | | 4 | 0.07 | ND | 0.32 | 0.08 | 0.09 | 0.12 | 0.12 | 0.80 |
| | | 28 | 5 | 0.08 | ND | 0.3 | 0.07 | 0.09 | 0.12 | 0.12 | 0.79 |
| | | | 6 | 0.08 | ND | 0.31 | 0.07 | 0.08 | 0.13 | 0.13 | 0.79 |
| | 50° C. | 14 | 7 | 0.07 | ND | 0.30 | 0.07 | 0.09 | 0.13 | 0.12 | 0.79 |
| | | | 8 | <0.05 | 0.67* | 0.29 | 0.07 | 0.09 | 0.12 | 0.12 | 1.37 |
| | | 28 | 9 | 0.07 | ND | 0.31 | 0.07 | 0.09 | 0.12 | 0.12 | 0.79 |
| | | | 10 | 0.07 | ND | 0.32 | 0.09 | 0.10 | 0.12 | 0.13 | 0.83 |
| Hard | n/a | initial | 1 | 0.06 | ND | 0.26 | 0.05 | 0.10 | 0.08 | 0.08 | 0.62 |
| Gelatin | | | 2 | 0.05 | ND | 0.24 | 0.05 | 0.10 | 0.07 | 0.08 | 0.58 |
| | 25° C. | 14 | 3 | 0.05 | ND | 0.22 | 0.05 | 0.10 | 0.07 | 0.07 | 0.56 |
| | | | 4 | <0.05 | ND | 0.18 | <0.05 | 0.09 | 0.07 | 0.08 | 0.42 |
| | | 28 | 5 | 0.05 | ND | 0.18 | <0.05 | 0.08 | 0.07 | 0.08 | 0.47 |
| | | | 6 | <0.05 | ND | 0.14 | <0.05 | 0.09 | 0.06 | 0.07 | 0.36 |
| | 50° C. | 14 | 7 | <0.05 | ND | 0.14 | <0.05 | 0.09 | 0.06 | 0.07 | 0.37 |
| | | | 8 | <0.05 | ND | 0.12 | <0.05 | 0.09 | 0.06 | 0.07 | 0.34 |
| | | 28 | 9 | <0.05 | ND | 0.11 | <0.05 | 0.09 | 0.06 | 0.07 | 0.33 |
| | | | 10 | <0.05 | ND | 0.09 | <0.05 | 0.11 | 0.06 | 0.07 | 0.33 |

*Appears to be a contamination peak since this high area at RRT 0.97 is not seen in the other replicate at this condition or at the later timepoint at the same (50 ° C.) storage condition.

### Part 3—Stability of Active Ingredient in Prototypes Produced by Wet Granulation

This part focuses on studying the stability of different blends granulated using different aqueous active ingredient solutions. Each granule was made as follows:
Solutions of the active ingredient (at 0.67 mg/mL) were prepared by dissolving 6.7 mg in 10 mL ethanol.
The granules were made by gradually adding 1.25 mL of the active ingredient solution into 5 g of a bed of the dry excipients (except the Mg stearate or Pruv lubricants) in a mortar, while using a pestle to stir.
Equivalent placebo granules were made where the same amount of solvent was added without the active ingredient dissolved.
The granules were left overnight to dry on the mortar on a warm hotplate. A sample of water filled into an identical mortar on the hotplate at the same setting was measured to reach a temperature of 37.6° C.
After drying, the required quantity of magnesium stearate was added across the granule in the mortar and the pestle used to achieve a homogeneous mix, without grinding.
The blends were then dispensed into vials (10 active vials and 3 placebo vials) using a 5-dp balance and placed on stability. The mass of granule in each vial was used to calculate the active ingredient assay values determined by HPLC analysis.

Table 12 details the composition of the four granules manufactured.

TABLE 12
Compositions of the Prototype Granulation Batches

| Active | pH 3 Citric | 5% aq. SLS | 0.1M HCl | 0.1M HCl |
|---|---|---|---|---|
| ingredient | Acid Buffer | | | |
| dissolved in | | | | |
| PGF (Primary | | | | |
| Granulation | | | | |
| Fluid) | | | | |
| Mass of sample | 270--330 mg | | | |
| per stability test | | | | |
| vial | | | | |
| Ingredient | Qty per ~5 g Batch | | | |
| Active | (~0.83 mg) | (~0.83 mg) | (~0.83 mg) | (~0.83 mg) |
| Ingredient | via 1.25 ml of | via 1.25 ml of | via 1.25 ml of | via 1.25 ml of |
| | PGF | PGF | PGF | PGF |
| Mannitol | 4.45 g | 4.45 g | 4.45 g | |
| Avicel PH101 | | | | 4.5 g |
| Explotab | 0.2 g | | | |
| (sodium starch | | | | |
| glycolate) | | | | |
| AcDiSol | | 0.2 g | | |
| (croscarmellose | | | | |
| sodium) | | | | |
| HPC-LS | | | 0.2 g | 0.2 g |
| Kollidon 25 | | 0.25 g | | |
| (Povidone, K25) | | | | |
| HPMC | 0.25 g | | 0.25 g | 0.25 g |
| Mg stearate | | | | 0.05 g |
| Pruv | 0.10 g | 0.10 g | 0.10 g | |
| Secondary | pH 3 citric acid | 5% aq. SLS | 0.1M HCl | 0.1M HCl |
| Granulation | buffer | | | |
| Fluid (if/as | | | | |
| required to | | | | |
| reach | | | | |
| granulation | | | | |
| endpoint) | | | | |

### Results

The assay values of the incubated samples of each of the wet granulated prototype formulations are summarised in Table 13. The areas of the impurity peaks for the corresponding samples are shown in Table 14, Table 15, Table 16, and Table 17 for each of the four formulations. It should be noted that the LOQ (limit of quantification) is only around 0.20% of the peak area in these runs, so values below this will be of low accuracy. From these data, it can be seen that:
Replicate agreement is generally poor across the samples i.e. there are significant differences in the active ingredient assay between the pairs of replicates at each timepoint/condition. These values have been corrected for the weight of the sample tested. Hence the variability suggests that the active ingredient uniformity is poor in these samples, perhaps the result of the low dose of the active ingredient and the small batch sizes prepared (difficult to achieve uniformity). However there is no trend of active ingredient assay decreasing with incubation time or temperature i.e. there is no evidence of gross degradation.
In the majority of cases, there is no change in the relative peak areas of the impurities with increasing incubation time or temperature i.e. no evidence of chemical degradation of the active ingredient. In fact most peaks are also seen at the same area in the active ingredient standard so are not true degradants.
   The one notable exception is the RRT 1.84 impurity whose area increased markedly with incubation time and temperature. After 28 days at the accelerated stability condition (50° C.), the peak area is higher than would be acceptable (<1% of peak area) for an unknown impurity by ICH Q3B guidelines. Hence formulation 0.1 M HCl Avicel is ruled out from further consideration.
   Formulation 0.1 M HCl Avicel also showed two other peaks (RRT 0.78 & 0.92) not seen in the API standard although these peak areas are barely detectable.
For formulation pH 3 citric acid buffer there is an anomalous increasing trend of assay with sample number in the LS sequence order i.e. from 1 to 10. Hence an apparent increase in assay with increasing incubation time and temperature. The areas of the impurities grow correspondingly (i.e. there is no trend of the impurity peak area relative to the active ingredient from Sample 1 to 10). The area of the active ingredient in the Standard solution also increased across this sequence, the peak symmetry worsened and the column pressure increased, believed to be from granule particles increasingly interfering with the chromatography. Hence the absolute peak areas are not considered reliable in this case.

TABLE 13
Assay of incubated samples of prototype granulation

| | | | Assay (% of nominal) | | | |
|---|---|---|---|---|---|---|
| | Storage | | pH3 citric | | | |
| | temperature | #Days | acid | | 0.1 M HCl | 0.1 M HCl |
| Sample # | (° C.) | incubation | Buffer | 5% aq. SLS | Mannitol | Avicel |
| 1 | Initial | | 86.5 | 104.0 | 74.0 | 106.4 |
| 2 | | | 86.2 | 78.3 | 86.2 | 109.6 |
| 3 | 25 | 14 | 92.3 | 87.3 | 91.7 | 109.4 |
| 4 | | | 98.4 | 95.7 | 81.5 | 106.7 |
| 5 | | 28 | 113.4 | 92.6 | 67.0 | 107.8 |
| 6 | | | 112.1 | 96.2 | 67.7 | 110.5 |
| 7 | 40 | 14 | 120.2 | 86.9 | 83.8 | 106.6 |
| 8 | | | 111.1 | 89.8 | 85.0 | 97.5 |
| 9 | | 28 | 127.3 | 89.3 | 81.7 | 107.0 |
| 10 | | | 128.1 | 91.1 | 102.0 | 111.6 |

| | | | | |
|---|---|---|---|---|
| Mean (across Samples #1 to 10) | 107.6 | 91.1 | 82.1 | 107.3 |
| RSD (across Samples #1 to 10, | 14.7 | 7.5 | 12.9 | 3.7 |
| %) | | | | |

TABLE 14
Peak areas of impurities of incubated samples for the pH3 citric acid buffer
prototype

| | Initial | | 14 days @ 25° C. | | 28 days @ 25° C. | | 24 days @ 50° C. | | 28 days @ 50° C. | |
|---|---|---|---|---|---|---|---|---|---|---|
| RRT | R1 | R2 | R3 | R4 | R5 | R6 | R7 | R8 | R9 | R10 |
| 0.95⁴ | 0.19 | 0.19 | 0.19 | 0.19 | 0.19 | 0.19 | 0.19 | 0.20 | 0.20 | 0.20 |
| 0.96⁴ | 0.12 | 0.12 | 0.11 | 0.11 | 0.11 | 0.11 | 0.11 | 0.12 | 0.11 | 0.11 |
| 1.06⁴ | 0.18 | 0.25 | 0.21 | 0.23 | 0.21 | 0.21 | 0.20 | 0.20 | 0.21 | 0.21 |
| 1.14⁴ | <0.05 | <0.05 | <0.05 | <0.05 | <0.05 | <0.05 | <0.05 | <0.05 | <0.05 | <0.05 |
| 1.26⁴ | 0.08 | 0.09 | 0.05 | 0.05 | 0.08 | 0.07 | 0.05 | 0.08 | 0.07 | 0.07 |
| Sum | 0.56 | 0.65 | 0.57 | 0.63 | 0.59 | 0.58 | 0.56 | 0.60 | 0.58 | 0.58 |

⁴the peak is also seen with the same area in the analytical standard

TABLE 15
Peak areas of impurities of incubated samples for the 5% aq. SLS prototype

| | Initial | | 14 days @ 25° C. | | 28 days @ 25° C. | | 24 days @ 50° C. | | 28 days @ 50° C. | |
|---|---|---|---|---|---|---|---|---|---|---|
| RRT | R1 | R2 | R3 | R4 | R5 | R6 | R7 | R8 | R9 | R10 |
| 0.95⁴ | 0.21 | 0.19 | 0.20 | 0.20 | 0.19 | 0.20 | 0.20 | 0.20 | 0.19 | 0.20 |
| 0.96⁴ | 0.12 | 0.09 | 0.10 | 0.11 | 0.10 | 0.10 | 0.10 | 0.10 | 0.10 | 0.20 |
| 1.06⁴ | 018 | 0.17 | 0.19 | 0.16 | 0.19 | 0.17 | 0.15 | 0.17 | 0.16 | 0.18 |
| 1.14⁴ | 0.06 | 0.06 | 0.06 | 0.07 | 0.06 | 0.06 | 0.85 | 0.06 | 0.06 | 0.06 |
| 1.26⁴ | 0.07 | 0.06 | 0,07 | 0.07 | 0.07 | 0,06 | 0.06 | 0.06 | 0.06 | 0.07 |
| Sum | 0.65 | 0.57 | 0.61 | 0.80 | 0.61 | 0.59 | 0.56 | 0.60 | 0.58 | 0.61 |

⁴the peak is also seen with the same area in the analytical standard

TABLE 16
Peak areas of impurities of incubated samples for the 0.1 M HCl Mannitol
prototype

| | Initial | | 14 days @ 25° C. | | 28 days @ 25° C. | | 24 days @ 50° C. | | 28 days @ 50° C. | |
|---|---|---|---|---|---|---|---|---|---|---|
| RRT | R1 | R2 | R3 | R4 | R5 | R6 | R7 | R8 | R9 | R10 |
| 0.95⁴ | 0.21 | 0.21 | 0.21 | 0.20 | 0.21 | 0.20 | 0.21 | 0.21 | 0.21 | 0.21 |
| 0.96⁴ | 0.12 | 0.12 | 0.12 | 0.12 | 0.13 | 0.11 | 0.11 | 0.12 | 0.12 | 0.11 |
| 1.06⁴ | 0.21 | 0.21 | 0.21 | 0.18 | 0.21 | 0.24 | 0.17 | 0.22 | 0.20 | 0.23 |
| 1.14⁴ | <0.05 | 0.05 | 0.05 | 0.05 | <0.05 | <0.05 | 0.05 | <0.05 | 0.05 | 0.05 |
| 1.27⁴ | 0.09 | 0.08 | 0.08 | 0.08 | 0.09 | 0.07 | 0.07 | 0.07 | 0.07 | 0.07 |
| Sum | 0.63 | 0.67 | 0.68 | 0.63 | 0.63 | 0.62 | 0.61 | 0.62 | 0.56 | 0.66 |

⁴the peak is also seen with the same area in the analytical standard

TABLE 17
Peak areas of impurities of incubated samples for the 0.1 M HCl Avicel
prototype

| | Initial | | 14 days @ 25° C. | | 28 days @ 25° C. | | 24 days @ 50° C. | | 28 days @ 50° C. | |
|---|---|---|---|---|---|---|---|---|---|---|
| RRT | R1 | R2 | R3 | R4 | R5 | R6 | R7 | R8 | R9 | R10 |
| 0.78 | <0.05 | <0.05 | <0.05 | <0.05 | <0.05 | <0.05 | <0.05 | <0.05 | 0.06 | <0.05 |
| 0.92 | <0.05 | <0.05 | <0.05 | <0.05 | <0.05 | 0.06 | <0.05 | <0.05 | <0.05 | <0.05 |
| 0.95⁴ | 0.21 | 0.21 | 0.21 | 0.21 | 0.19 | 0.17 | 0.20 | 0.18 | 0.19 | 0.19 |
| 0.96⁴ | 0.11 | 0.11 | 0.10 | 0.10 | 0.12 | 0.13 | 0.11 | 0.13 | 0.11 | 0.11 |
| 1.06⁴ | 0.26 | 0.26 | 0.21 | 0.22 | 0.29 | 0.27 | 0.23 | 0.26 | 0.20 | 0.22 |
| 1.14⁴ | <0.05 | 0.05 | <0.05 | <0.05 | <0.05 | <0.05 | <0.05 | <0.05 | <0.05 | <0.05 |
| 1.26⁴ | 0.07 | 0.07 | 0.07 | 0.06 | 0.07 | 0.06 | 0.05 | 0.05 | 0.05 | 0.06 |
| 1.84 | 0.86 | 0.20 | 0.41 | 0.26 | 0.62 | 0.42 | 1.01 | 1.11 | 1.65 | 0.94 |
| Sum | 1.01 | 0.90 | 1.18 | 0.86 | 1.30 | 1.13 | 1.50 | 1.76 | 2.28 | 1.53 |

⁴the peak is also seen with the same area in the analytical standard

### Conclusions

### Formulation Prototypes

The physical solubility of the active ingredient was measured through observations of undissolved active ingredient in six potential granulation fluids. It was found that the active ingredient was sufficiently soluble to enable wet granulated to be dosed at 200 μg per capsule for the following fluids with the smallest achievable capsule size indicated for each fluid:
0.1 M HCl and ethanol: [active ingredient]>7 mg/mL, i.e. suitable for 200 g in Capsule Size 4 or larger shells
Aq. 5% SLS and pH 3 Citric buffer: 3 mg/mL>[active ingredient]>0.6 mg/mL, i.e. suitable for 200 μg in Capsule Size 00 (but no smaller)

The stability of the active ingredient was evaluated for each of these four options during the potential drying time of 20-28 hours at ambient temperature—no active ingredient related impurities were seen and so all four fluids can be considered as options for wet granulation formulations. The active ingredient solubility in the remaining two fluids (50:50 EtOH:water or aq. 5% Tween 80) was below 0.6 mg/ml making them unable to deliver the higher 200 μg dose in any capsule size. Hence these two options were ruled out.

Of the four acceptable candidates, 0.1 M HCl was selected as the primary granulation fluid since it enables the delivery of both the 50 and 200 μg dose in the preferred capsule sizes (Size 2 to 4) while avoiding the use of non-aqueous solvents.

The 28-day stability of four prototype wet granulation formulations was evaluated. The ingredients in these formulations are listed in Table 12. Of these formulations, only the 0.1 M HCL Avicel prototype showed any evidence of active ingredient degradation increasing with time and temperature—this formulation can be discounted since one peak (RRT 1.84) exceeded ICH Q3B criteria for unknown impurities (i.e. was >1% of total area). The ingredients in this formulation which are not present in the other three formulations are Avicel and magnesium stearate. Hence these ingredients can be eliminated from future consideration in wet granulations for the active ingredient because of potential chemical incompatibilities whereas the other excipients in this formulation (0.1M HCl, HPC-LS and HPMC Methocel E5) have been shown to be chemically compatible with the active ingredient from the other three formulations. This, alone with the results for the other three formulations, suggests all the following are chemically compatible with the active ingredient in such formulations:
Aqueous granulation fluids: 0.1 M HCl, pH 3 Citric Acid Buffer and 5% SLS
Diluents: Mannitol
Disintegrants: sodium starch glycolate, HPC-LS and croscarmellose sodium
Binders: both HPMC and Povidone
Lubricants: sodium stearyl fumarate

### Capsule Shells

The 28 day stability of the active ingredient in HPMC and Gelatin capsule shells was evaluated. There was no evidence of active ingredient degradation for either HPMC or gelatin capsules. The % assay of the shells remained close to nominal over the 28 day incubation, with no impurity peaks increasing with incubation temperature or time. Therefore, both capsule showed compatibility with the active ingredient.

### Example 3—Evaluation of Binders for a Granule-In-Capsule Formulation

Two binders, PVP and HPMC, were studied at High and Low Granulation Extent. At Low Granulation Extent, the blend was granulated using Primary Granulation Fluid (PGF) and small volume of 0.1 M HCl with no dissolved active ingredient (Secondary Granulation Fluid (SGF)). At High Granulation Extent was made with same amounts of PGF, but more SGF was added to achieve further granulation extent. Although both binders show good stability and compatibility with the active ingredient as reported in Example 2, granules produced with HPMC showed superior active ingredient uniformity and fewer active ingredient losses, therefore HPMC was selected to produce the ICH stability batches.

When studied for the 50-μg dose (size 4 capsules), the dissolution rates were found to be similar at High and Low Granulation Extents. However, the High Granulation Extent showed better Content Uniformity than the Low Granulation Extent and so was selected for the ICH stability batches. The low dose, 50 μg, was filled into Size 4 capsules and the high dose, 200 μg, was made into size 00 capsules for the ICH batch, both Quali-G PEG (hard gelatin).

The appearance, assay, content uniformity, dissolution and microbiological analysis of the stability batches all met specification at T=0 with no LC-detectable degradation having occurred during manufacture.

### Materials Used

| | |
|---|---|
| Active Ingredient | Onyx (lot: JCM1019B) |
| Mannitol (Pearlitol 100 SD) | Roquette |
| L-HPC LS-B1 | Chemlink Harke |
| PVP (Kollidon K25) | BASF |
| HPMC (Methocel E5 Premium) | Colorcon |
| Pruv (Sodium Stearyl fumarate) | JRS Pharma |
| Red Size 4 Quali V (HPMC) Capsules | Qualicaps |
| Green/Grey Size 4 Quali - G PEG (hard | Qualicaps |
| gelatin) capsules | |
| White Size 2 Quali- V (HPMC) capsules | Qualicaps |
| White Size 2 Quali- G PEG (hard | Qualicaps |
| gelatin) capsules | |

### Analytical Testing

### Dissolution Testing

Dissolution testing in USP II vessels in 500 mL 0.1M HCl QC Media (or 500 mL pH 6.8 Phosphate buffer) at 37° C., at a paddle speed of 50 RPM (and 200 RPM infinity spin after 45 min sampling). 0.2 μm PVDF cannula filters and O-ring sinkers where used (4-5 mm for size 4 capsules and 6-7 mm for size 2 capsules)

### Content Uniformity

10 μL of 1.0 μg/mL, 25 μL of 0.25 μg/mL solutions of the diluent (90:10 v/v 0.1 M HCl:Acetonitrile) for high dose and low dose respectively.

### HPLC/UPLC Conditions for Dissolution and Content Uniformity

TABLE 18
UPLC/HPLC parameters used for dissolution testing

| Parameter | Value | | |
|---|---|---|---|
| Column | Halo C18 2.7 μm, 3.0 × 50 mm | | |
| Column temperature | 40° C. | | |
| Sample manager | 50:50 v/v HRLC Water, Acetonitrile | | |
| wash (UPLC) | | | |
| Sample manager | HPLC water + 0.1% Phosphoric Acid | | |
| purge (UPLC) | | | |
| Mobile Phase A | HPLC water + 0.1% Phosphoric Acid | | |
| Mobile Phase B | 100% Acetonitrile | | |
| | Time | Mobile | Mobile |
| | (min) | Phase A (%) | Phase B (%) |
| Gradient Program | 0 | 80 | 20 |
| | 0.5 | 80 | 20 |
| | 3 | 20 | 80 |
| | 3.01 | 2 | 98 |
| | 6 | 2 | 98 |
| | 6.01 | 80 | 20 |
| | 9.5 | 80 | 20 |

| Flow rate | 0.5 mL/min | |
|---|---|---|
| Run Time | 9.5 minutes | |
| Dose strength | Hugh Dose 200 μg | Low Dose 50 μg |
| Injection volume | 10 μL | 25 μL |

| | |
|---|---|
| Wavelength | 217 nm |
| 3D Data Range | 180 to 400 nm |
| Bandwidth (HPLC) | 10 |
| Sampling rate | 10 points/ sec |
| Peak Width (HPLC) | >0.05 min |

[symbol]

[symbol]

indicates data missing or illegible when filed

### Assay/Related Substances HPLC/UPLC Method Conditions

Assay (1) and related substances (2) testing were both carried using acetonitrile as the analytical diluent; (1) with a nominal concentration of 0.01 g/mL, while (2) were made to 0.025 mg/mL. The HPLC/UPLC conditions are given in Table 19.

TABLE 19
HPLC/UPLC parameters used for Assay
and related substance testing

| Parameter | Value | | |
|---|---|---|---|
| Column | Halo C18 2.7 μm, 3.0 × 50 mm 92813-402) | | |
| Column Tempurature | 40° C. | | |
| Sample manager | HPLC Water: ACN 50:50% v/v | | |
| wash (UPLC) | | | |
| Sample manager | HPLC Water: ACN 50:50% v/v | | |
| purge (UPLC) | | | |
| Mobile phase A | 10 mM Ammonium Acetate pH 7.0 | | |
| Mobile phase B | Acetonitrile | | |
| | Time | Mobile | Mobile |
| | (minutes) | Phase A (%) | Phase B (%) |
| Gradient program | 0 | 95 | 5 |
| | 2 | 55 | 45 |
| | 7.1 | 50 | 50 |
| | 9.7 | 18 | 82 |
| | 10.3 | 2 | 98 |
| | 21 | 2 | 98 |
| | 21.1 | 95 | 5 |
| | 26 | 95 | 5 |

| Flow rate | 0.3 mL/min | |
|---|---|---|
| Run time | 26 minutes | |
| Solution for injection | Solution In ACN | HPLC water |
| | | Auto-Addition |
| Injection volume | 12 μL | 38 μL |

| | |
|---|---|
| Wavelength | 256 nm |
| 3D data range | 180 to 400 nm |
| Sampling rat | 10 points/ sec |

### Part 1—Impact of Binder Type and Granulation Extent on Blend Uniformity

Four granule batches were prepared. In summary the preparation method was as follows:
i. The PGF (Primary Granulation Fluid) was prepared by dissolving the active ingredient in 500 mL of 0.1 M HCl by stirring for 10 minutes.
ii. The required quantities of binder (HPMC/PVP), mannitol and L-HPC were added to the Multipro high shear mixer and mixed at high speed for 5 min.
iii. While still mixing at high speed, the quantities of PGF and SGF (Secondary Granulation Fluid=0.1 M HCl) were added as summarised in Table 20 until the required granulation endpoints were reached.
   The SGF was used to rinse out the PGF dispense container to ensure no loss of API.
   Note that the amounts of SGF added were deliberately varied between the pair of HPMC and PVP batches to evaluate the high and low extents of granulation.
iv. The batches were dried on a warmed tray.
v. Sampling of the batches was performed for blend uniformity testing to create two sets of samples:
   “Non-sieved” where the granule batches were split down to 3 g using a sampling splitting box and then ~200 mg samples weighed into vials for analysis
   “Fines” where ~200 mg samples were taken material passing through a 125 μm mesh (using a sieve shaker at 1 mm amplitude for 5 minutes).

Table 20 describes the quantities of active ingredient used to prepare the compositions. The compositions of granules made with HPMC and PVP at Low and High Granulation Extent is summarised in Table 21.

TABLE 20
Quantities per batch

| Material | | | | | |
|---|---|---|---|---|---|
| Active | Primary | 50.0 | 50.0 | 50.0 | 50.0 |
| Ingredient | granulation | | | | |
| | fluid | | | | |
| | containing | | | | |
| | active | | | | |
| | ingredient | | | | |
| | (g) | | | | |
| | Active | 0.1801 | 0.1800 | 0.1799 | 0.1800 |
| | Ingredient | | | | |
| | (0.36% of | | | | |
| | total weight | | | | |
| | of Primary | | | | |
| | Granulation | | | | |
| | Fluid) (g) | | | | |

| | | | | |
|---|---|---|---|---|
| Mannitol (Pearlitol | 187.5 (182.8)* | 187.5 (182.8)* | 87.5 (182.8)* | 187.5 (182.8)* |
| 100 SD) (g) | | | | |
| L-HPC LS-B1 (g) | 8.21 (8.0)* | 8.20 (8.0)* | 8.20 (8.0)* | 8.21 (8.0)* |
| HPMC (Methocel E5 | 5.11 (5.0)* | 5.11 (5.0)* | | |
| Premium) (g) | | | | |
| Kollidon 25 (g) | | | 5.10 (5.0)* | 5.10 (5.0)* |
| Dry Total | 201 (196)* | 201 (196)* | 201 (196)* | 201 (196)* |
| Secondary Granulation | 15 | 5 | 15 | 5 |
| Fluid (mL) | | | | |

*The values outside the brackets are the quantities dispensed, while the values in brackets are theoretical amounts after taking a sample of 5 g for LOD test.

TABLE 21
Granule batches compositions

| | | | |
|---|---|---|---|
| High HPMC | Low HPMC | High PVP | Low PVP |

| Ingredient | % w/w composition | |
|---|---|---|
| Active ingredient | 0.09% | 0.09% |
| Pearlitol 100SD | 92.3% | 92.3% |
| (Mannitol) | | |
| L-HPC LS-B1 | 4.0% | 4.0% |
| Kollidon 25 (PVP) | — | 2.5% |
| Methocel E5 (HPMC) | 2.5% | — |
| Total (Granule | 99% | 99% |
| before lubrication) | | |
| Sodium Stearyl | 1.0% | 1.0% |
| fumarate | | |

### Results

Granules were successfully produced. Granulation process is summarised in Table 22. Visually, all granules (high and low granulation extents) appear to be uniform with low fines levels. Increasing the granulation extent visually reduced the amount of fines for both binders as expected. The LOD was consistent across all four batches.

TABLE 22
Granulation process summary for Part 1

| | | Primary | Secondary | Total volume | | |
|---|---|---|---|---|---|---|
| Extract of | | Granulation | granulation | of fluid | | |
| granulation | Binder | fluid (mg) | fluid (g)* | added (g)* | Granule Appearance | LOD % |
| High | HPMC | 50 | 15 | 65 | White granular powder | 0.49 |
| | | | | | ~98% granules^{§} | |
| | PVP | 50 | 15 | 65 | White granular powder | 0.73 |
| | | | | | ~98% granules^{§} | |
| Low | HPMC | 50 | 0 | 50 | White fine granular powder | 0.68 |
| | | | | | ~90% granules^{§} | |
| | PVP | 50 | 0 | 50 | White fine granular powder | 0.48 |
| | | | | | ~90% granules^{§} | |

*This was recorded as a volume and converted to weigh (the density of 0.1M HCl is 1g/mL)
^{§}A visual estimate of the relative proportion of granules vs fines was made where “granules” were defined as having a larger particle size than the starting material.

From this study and Table 23 the following can be seen:
All granule batches except that made with PVP at High Granulation Extent (PH) showed good blend uniformity i.e. AV is ≤15.0
   The magnitude of AV is mainly governed by differences in the RSD of active ingredient content here. The RSD was unfavorably high at the High Granulation Extent for both binders, but acceptably low at the Low Granulation Extent.
   Note for PH: The high value (20.3) for AV is caused by one capsule with an active ingredient content of 117.3% that is responsible for the very high RSD.
The mean active ingredient content was below nominal throughout, and
   Closer to nominal at the High Granulation Extent compared to the Lower Granulation Extent for both binders.
   Closer to nominal with HPMC than PVP at both granulation extents.

TABLE 23
Blend uniformity results of granule batches (Part 1)

| HPMC binder (Low | | | HPMC binder (High | | PVP binder (Low | | PVP binder (High | |
|---|---|---|---|---|---|---|---|---|
| Granulation Extent) | | | Granulation Extent) | | Granulation Extent) | | Granulation Extent) | |
| [symbol] | CL-BU1 | 92.7 | CH-BU1 | 108.0 | PL-BU1 | 94.1 | PH-BU | 100.0 |
| | CL-BU2 | 93.9 | CH-BU2 | 100.6 | PL-BU2 | 94.9 | PH-BU | 96.4 |
| | CL-BU3 | 97.1 | CH-BU3 | 94.5 | PL-BU3 | 92.6 | PH-BU | 93.5 |
| | CL-BU4 | 94.3 | CH-BU4 | 91.8 | PL-BU4 | 94.7 | PH-BU | 95.9 |
| | CL-BU5 | 98.1 | CH-BU5 | 93.6 | PL-BU5 | 99.5 | PH-BU | 95.8 |
| | CL-BU6 | 94.2 | CH-BU6 | 93.4 | PL-BU6 | 96.7 | PH-BU | 93.0 |
| | CL-BU7 | 92.0 | CH-BU7 | 96.2 | PL-BU7 | 94.2 | PH-BU | 91.0 |
| | CL-BU8 | 96.0 | CH-BU8 | 100.7 | PL-BU8 | 92.0 | PH-BU | 91.8 |
| | CL-BU9 | 92.9 | CH-BU9 | 94.6 | PL-BU9 | 93.1 | PH-BU | 117.2 |
| | CL-BU10 | 95.3 | CH-BU10 | 101.0 | PL-BU10 | 92.7 | PH-BU | 90.6 |
| | Mean | 94.7 | Mean | 97.8 | Mean | 94.4 | Mean | 96.7 |
| | Min | 92.0 | Min | 91.8 | Min | 92.0 | Min | 90.6 |
| | Max | 99.1 | Max | 108.0 | Max | 99.5 | Max | 117.3 |
| | Standard | 2.0 | Standard | 4.9 | Standard | 2.2 | Standard | 7.3 |
| | Deviation | | Deviation | | Deviation | | Deviation | |
| | RSO | 2.20% | RSO | 5.00% | RSO | 2.40% | RSO | 8.00% |
| | AV | 9.0 | AV | 12.3 | AV | 9.4 | AV | 20.3 |

[symbol]

indicates data missing or illegible when filed

As well as assessing the uniformity of the active ingredient in the granule, the % active ingredient and uniformity in fine particles (this are defined as the particles <125 μm) were assessed. From the results in Table 24, it can be seen that:
The amount of fines was generally uniform in all granules and the quantities correspond to that in the granules. This indicates that active ingredient was well distributed across all size fractions in the granule with one exception:
   At High Granulation Extent, HPMC demonstrates the highest proportion of fines: 110.5%, i.e. notably higher than the mean result (without sieving) in Table 23.

Therefore, the active ingredient is uniformly distributed in the blend, which will assure acceptable dose uniformity.

Overall, in terms of the Blend Uniformity, HPMC showed superior results than the PVP, at least at the Low Granulation Extent. HPMC was nominated to be the binder used in the later studies. While the results at this stage suggested that the Low Extent of granulation might give superior blend uniformity, blend uniformity is not the only response of interest. The Extent of Granulation with HPMC was further studied (see Part 2), in order to gain more knowledge on the performance of the capsules in terms of dissolution and to assess the content uniformity of the active ingredient in capsules at High and Low Granulation Extent.

TABLE 24
Blend uniformity of fines

| Granulation Extent | Repeat | % Assay |
|---|---|---|
| Low Granulation | CL-BUF11 | 93.8 |
| Extent HPMC | CL-BUF12 | 90.3 |
| | Mean | 92.1 |
| High Granulation | CH-BUF11 | 107.6 |
| Extent HPMC | CH-BUF12 | 113.4 |
| | Mean | 110.5 |
| Low Granulation | PL-BUF11 | 96.9 |
| Extent PVP | PL-BUF12 | 94.0 |
| | Mean | 95.4 |
| High Granulation | PH-BUF11 | 92.7 |
| Extent PVP | PH-BUF12 | 93.0 |
| | Mean | 92.9 |

### Part 2—Study of the Extent of Granulation on Dissolution and Content Uniformity of Capsules

For this study, a total of four granule batches were made, two actives and two placebos each at 350 g. One active and a matching placebo batch were granulated for each granulation extent. For the ICH Study a total of four active sublots were made and eight Sublots for the placebo.

The different between Low Extent and High Extent is the addition of further SGF to make the resultant material less fine/more granular. The active batches were granulated in a humidity-controlled environment (15-25% RH), while the placebo (diluent) batches were granulated and dried at ambient humidity. The PGF (i.e. primary granulation fluid) was made by dissolving the active ingredient in 0.1 M HCl at 0.49% w/w. Each granule sublot was made as per Table 25 and Table 27 as follows:
i. All the excipients/active ingredient (except the lubricant) were dispensed into the high shear Multipro and blended dry for approximately 5 mins.
ii. The required volume of PGF (primary granulation fluid) was added over 15 minutes, as per Table 27, at a steady rate (~5-6 mL/min) while continuing to mix at high speed
iii. Further SGF (secondary granulation fluid, i.e. 0.1M HCl) was added as per Table 27. For each of the active and placebo batches, two different levels of granulation fluid were added so as to investigate the effect of granulation extent on the properties of the finished drug product:
   Low level (10 mL)—I=Low Granulation Extent, added over 2 minutes to the High Granulation Extent Batch and over 3 minutes to the Low Granulation Extent Batch
   High Level (15 mL)—High Granulation Extent added over 8 minutes
iv. Granules were dried on as a thin layer on trays at 60° C. on a hotplate

At this point in Part 2, two blends in Table 26 were made from the Sublots in Table 27 using the following method:
i. The required mass of active granule was determined based on the active ingredient dose and the mass of the diluent was determined based on the density of the granule.
ii. The Erweka AR401 was used to blend the active and diluent granules for 15 minutes at 15 rpm followed by 5 minutes at the same speed after addition of the lubricant
iii. Profiller 1100 was set up with Size 4 capsules
iv. The mass of the active granule was dispensed and added to the profiler.
   The quantity per plate was determined by calculating the mass of active granule required to deliver the desired dose per capsules (50 μg in this case) and therefore was scaled up to for 100 capsules. Based on the density of the granule and the volume of the capsule, the required diluent to fill the capsule was calculated.
v. The granule was evenly spread into the capsule using spreader until the capsules are level-filled
vi. The blend was temped as required until all the blend was filled in the capsules
vii. The dissolution of the capsules was carried out and based on the dissolution profile the granule type was chosen

TABLE 25
Formulation composition for active and placebo
granules for development and ICH batches

% w/w

| Material | Active | Placebo |
|---|---|---|
| Active Ingredient | 0.1213^{$} | Nil |
| Mannitol (Pearlitol | 92.4 | 92.5 |
| 100 SD) | | |
| L-HPC LS-B1 | 4.00 | 4.00 |
| HPMC (Methocel | 2.50 | 2.50 |
| E5 Premium) | | |
| Sodium Stearyl fumarate | 1.000 | 1.000 |

^{$}Corrected (from the theoretically pure % w/w of active ingredient of 0.12%) based on 98.2% potency

TABLE 26
Blend and Capsule Composition

| | TRI002/89G50-4B | | | TRI002/89G50-4A | | |
|---|---|---|---|---|---|---|
| | Low Extent of Granulation | | | High Extent of Granulation | | |
| | | | Density | | | Density |
| Material | g/Batch | % w/w | (g/mL)^{$} | g/Batch | % w/w | (g/mL)^{$} |
| Active granule | 6.42 | 44.4 | 0.40 | 6.25 | 41.1 | 0.43 |
| Diluent granule | 7.89 | 54.6 | | 8.79 | 57.9 | |
| Sodium | 0.1446 | 1.000 | | 0.152 | 1.000 | |
| Stearyl | | | | | | |
| fumarate | | | | | | |
| Total | 14.45 | 100.0 | | 15.20 | 100.0 | |

Excipient/Active Ingredient Composition

| Material | % w/w | mg/capsule | % w/w | mg/capsule |
|---|---|---|---|---|
| Active | 0.0538 | 0.050 | 0.0499 | 0.051 |
| Ingredient | | | | |
| Mannitol | 92.4 | 86.7 | 92.5 | 93.7 |
| (Pearlitol 100 | | | | |
| SD) | | | | |
| L-HPC LS-B1 | 4.00 | 3.75 | 4.00 | 4.05 |
| HPMC | 2.50 | 2.34 | 2.50 | 2.53 |
| (Methocel E5 | | | | |
| Premium) | | | | |
| Sodium | 1.000 | 0.938 | 1.000 | 1.013 |
| Stearyl | | | | |
| fumarate | | | | |
| Total | 100.0 | 93.8 | 100.0 | 101.3 |

| | | |
|---|---|---|
| Total number | 1 at 2 mm depth | 1 at 2 mm depth |
| of Temps | | |
| Capsule | Size 4 Quali - V (Red Opaque) | Size 4 Quali - V (Red Opaque) |
| details | | |

^{$}Bulk density of the sublot 1 of the granule batches

TABLE 27
Summary of granulation process (Granule compositions
for each batch number are detailed in Table 32)

| | | Granulation Fluid (g)\hat{ } | | Drying time | Final |
|---|---|---|---|---|---|
| Batch Code | Sublot | Primary | Secondary | (hours:min) | Appearance |
| TRI002/8AGA | 1 | 87.5 | 14.98 | 02:00 | Granular white and |
| | 2 | 87.3 | 14.94 | | homogenous with fines |
| | 3 | 87.3 | 14.92 | | |
| | 4 | 87.3 | 14.92 | | |
| TRI002/8DGA | 1 | 87.6 | 14.81 | 16:56 | Granular white and |
| | 2 | 87.4 | 15.03 | | homogenous with fines |
| | 3 | 87.3 | 14.96 | | and few granules |
| | 4 | 87.1 | 14.98 | | larger than 2 mm |
| | 5 | 87.4 | 14.98 | 40:48 | |
| | 6 | 87.4 | 14.97 | | |
| | 7 | 87.1 | 14.92 | | |
| | 8 | 87.1 | 14.99 | | |
| TRI002/8AGB^{¥} | 1 | 87.0 | 10.00 | NR* | Granular white and |
| TRI002/8DGB | 1 | 87.4 | 9.97 | 12:57 | homogenous with fines |

*NR—Not recorded
^{¥}Values for these batches are the target values
\hat{ }The primary granulation fluid (PGF) consisted of 0.1M HCl with dissolved active ingredient at 0.49% w/w, while the secondary granulation fluid (SGF) consisted of only 0.1M HCl

### Results

### Encapsulation:

All granules (High and Low Extent of Granulation) were successfully produced. The characteristics are summarised in Table 28 and Table 29. The granules appear to be white granular powder with different proportions of fines (more fines in the Lower Extent of Granulation, as expected). Granule A had a higher density (bulk and tap) than Granule B. However all the other quantifiable characteristics are essentially the same between the two batches:
The Carr's index, Hausner Ratio and flow rate of Granule A are consistent with granule B.
The PSD of Option A is consistent with Option B.

TABLE 28
Characterisation results for Granule A and Granule B placebo

| | | | |
|---|---|---|---|
| Test | Rep | TRI002/89DGA | TRI002/89DGB |

| | | |
|---|---|---|
| Granulation Extent | High | Low |

| Appearance | | | white | white granular |
|---|---|---|---|---|
| | | | granules | powder |
| | Bulk Density | 1 | 0.43 | 0.39 |
| | (g/mL) | 2 | 0.44 | 0.41 |
| | | Mean | 0.43 | 0.40 |
| | Tap density | 1 | 0.51 | 0.44 |
| | (g/mL) | 2 | 0.50 | 0.47 |
| | | Mean | 0.50 | 0.45 |
| | Carrs Index | 1 | 15.0 | 9.7 |
| | (%) | 2 | 13.0 | 12.0 |
| | | Mean | 14.0 | 10.9 |
| | Hausner | 1 | 1.18 | 1.11 |
| | Ratio | 2 | 1.14 | 1.14 |
| | | Mean | 1.16 | 1.13 |
| | Flow | 1 | 5.3 | 4.6 |
| | through | 2 | 5.0 | 4.7 |
| | 10 mm (g/s) | Mean | 5.1 | 4.7 |
| | Flow | 1 | 13.8 | 11.6 |
| | through | 2 | 12.0 | 11.8 |
| | 25 mm (g/s) | Mean | 12.9 | 11.7 |

TABLE 29
PSD for Granule A and Granule B placebo

Granulation Extent

| | High | Low |
|---|---|---|
| Mesh size (μm) | TRI002/89DGA | TRI002/89DGB |
| 1000 | 0.7 | 0.0 |
| 710 | 8.6 | 2.9 |
| 500 | 5.4 | 3.6 |
| 355 | 4.6 | 3.3 |
| 250 | 5.8 | 4.8 |
| 125 | 31.7 | 48.1 |
| 63 | 37.3 | 34.6 |
| Pan | 6.0 | 2.7 |
| Total | 100.1 | 99.8 |

The corresponding active and diluent granule batches were blended as Table 26 and Table 27. Each blend was successfully encapsulated into size 4 capsules to with the required mass to achieve a theoretical 50 μg dose i.e. giving a set of −4A capsules from a High Extent and a −4B set with a Low Extent of Granulation.

The density of each granule was taken into account when determining the fill weights. As in Table 30, the −4A capsules demonstrated comparable RSD of capsule weights to −4B. Both −4A and −4B capsules were close to the target weight. However, the mean weight of −4A capsules were slightly closer to the target weight than −4B. While a slight underfilling was observed during encapsulation of −4A capsules, the fill weight was correct to achieve the target dose for the 50 μg ICH Stability batch.

TABLE 30
Statistics for the individual capsule weights

| | Active | |
|---|---|---|
| | TRI002/89G50-4A | TRI002/89G50-4B |
| Granulation Extent | High | Low |
| Target fill weight (mg) | 101.3 | 93.8 |
| Average fill weight (mg) | 101.0 | 92.1 |
| Average fill weight | 99.7 | 98.2 |
| (% of Target) | | |
| SD | 2.9 | 3.3 |
| % RSD | 2.9 | 3.6 |

### Dissolution

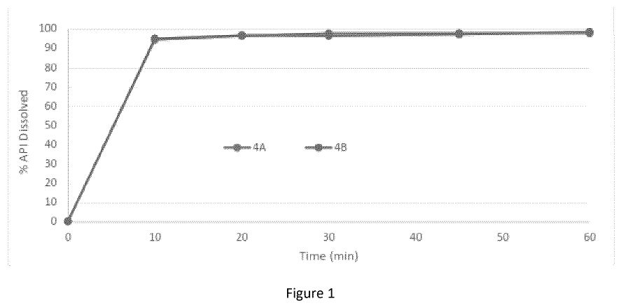

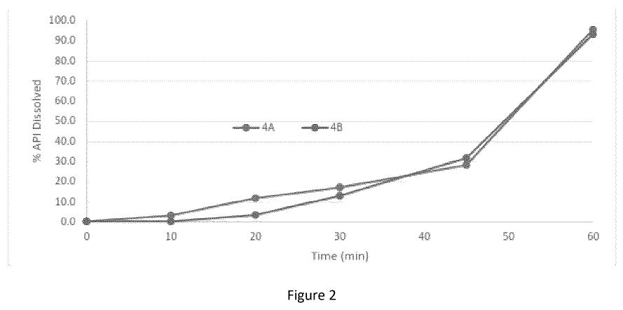

The dissolution rate in 0.1 M HCl of both granules, A and B capsules were identical (FIG. 1). Therefore, granulation extent has no impact on the dissolution rate in 0.1 M HCl media. While the dissolution rate of Option A was initially faster than Option B in pH 6.8 phosphate buffer (FIG. 2), this difference can be discounted because:
Regardless of granulation extent, the dissolution rate was extremely slow over the first 45 minutes at 45 RPM and incomplete after the final 15 minutes at 200 RPM.
The slow and incomplete dissolution rate is due to the slow dissolution of the HPMC capsules caused by the presence of the potassium ions in the media. Because of this (and a reduction in the LC interference), hard gelatin capsules were used in place of HPMC for the ICH stability batches—their disintegration is not slowed by potassium ions.

### Content Uniformity

Capsules containing granule Option A and B give consistent and acceptable content uniformity, i.e. AV<15 (Table 31). As expected (from the effects of the blending and encapsulation process), the AV values are slightly higher than the Blend Uniformity values in Part 1 (Table 23). The trends in AV are explained by two factors:
RSD of active ingredient Content: The key advantage to Option A (High Granulation Extent) was its provides superior uniformity (low RSD of 2.5 in contrast to the opposite trend in the Blend Uniformity data in Part 1 (Table 23)
Proximity of active ingredient content to the target: Both Options, A and B give a mean active ingredient content value further from target (100%) and lower than Part 1. However, the mean active ingredient % of Option B is slightly higher than A, i.e. closer to target. However that advantage for B is less of a concern:
   The lower capsule fill weight of Option A has contributed to the lower mean content value.
   The mean active ingredient content of Option A is likely to improve with scale up (less active ingredient losses),
With that advantage for B demoted, Option A is preferred over B in terms of its superior Uniformity (discussed above)

The above rationale concerning content uniformity was the deciding factor for selecting Option A (High Granulation Extent) for making the ICH Stability batches.

TABLE 31
content uniformity of Option A capsules Vs Option B capsules

| | | | Assay | | | | |
|---|---|---|---|---|---|---|---|
| | | | % re- | | Std | | |
| Batch | Repli- | Weight | covery | Mean | Dev | % | Acceptance |
| Code | cate | (mg) | (T) | (X) | (s) | RSO | value (AV) |
| TRI002/ | Rep 1 | 45.5 | 91.0 | 92.5 | 2.5 | 2.7 | 12.0 |
| 89G50- | Rep 2 | 47.7 | 95.4 | | | | |
| 4A | Rep 3 | 44.7 | 89.3 | | | | |
| (50 μg) | Rep 4 | 46.0 | 92.0 | | | | |
| | Rep 5 | 45.9 | 91.8 | | | | |
| | Rep 6 | 45.5 | 91.1 | | | | |
| | Rep 7 | 45.5 | 91.1 | | | | |
| | Rep 8 | 46.2 | 92.3 | | | | |
| | Rep 9 | 49.0 | 98.0 | | | | |
| | Rep 10 | 46.6 | 93.3 | | | | |
| TRI002/ | Rep 1 | 46.9 | 93.9 | 94.4 | 3.6 | 3.8 | 12.7 |
| 89G50- | Rep 2 | 47.8 | 95.6 | | | | |
| 4B | Rep 3 | 48.8 | 97.6 | | | | |
| (50 μg) | Rep 4 | 43.3 | 86.6 | | | | |
| | Rep 5 | 46.5 | 92.9 | | | | |
| | Rep 6 | 47.3 | 94.6 | | | | |
| | Rep 7 | 46.3 | 92.5 | | | | |
| | Rep 8 | 46.7 | 93.4 | | | | |
| | Rep 9 | 48.5 | 97.1 | | | | |
| | Rep 10 | 49.9 | 99.9 | | | | |

### Part 3: Preparation of ICH Stability Batches

Based on the outcome of the high and low granulation extent study (Table 32), a granulation extent option was selected as discussed in Part 2. The reminder of the granule sublots were made and all sublots were mixed and lubricated before encapsulated into size 2 and size 4 Quali-G PEG capsules to produce 200 μg and 50 μg dose capsules respectively (Table 33). Matching placebos were also made (Table 34).

TABLE 32
Granule composition during Part 2 and the ICH Stability Batches

| | g/batch | | | | | |
|---|---|---|---|---|---|---|
| | TRI002/8AGA | | TRI002/8DGA | | TRI002/ | TRI002/ |
| | Sublot | Sublots | Sublot | Sublots | 8AGB§ | 8DGB |
| Material | 1 | 2-4 | 1 | 2-4 | Sublot 1 | Sublot 1 |
| Active | 0.423 | 1.334 | 0.00 | 0.00 | 0.425 | 0.00 |
| Ingredient* | | | | | | |
| Mannitol | 323 | 1018 | 324 | 2380 | 323 | 324 |
| (Pearlitol | | | | | | |
| 100 SD) | | | | | | |
| L-HPC | 14.02 | 44.1 | 14.02 | 103.0 | 14.00 | 14.01 |
| LS-B1 | | | | | | |
| HPMC | 8.77 | 27.6 | 8.75 | 64.3 | 8.75 | 8.77 |
| (Methocel | | | | | | |
| E5 | | | | | | |
| Premium | | | | | | |

*Dissolved in primary granulation fluid (0.1 M HCl) - see quantities in Table 27
§Target (not actual) values quoted

TABLE 33
Blend and Capsule composition used for the active ICH Stability Batches

| | TRI002/89G50-4A | | TRI002/89G200-2H | |
|---|---|---|---|---|
| Material | g/Batch | % w/w | g/Batch | % w/w |
| Active | 82.93 | 33.3 | 332 | 85.5 |
| (TRI002- | | | | |
| 89AGA) | | | | |
| Diluent | 163.3 | 65.7 | 52.3 | 13.50 |
| (TRI002- | | | | |
| 89AGD) | | | | |
| Sodium Stearyl | 2.49 | 1.000 | 3.88 | 1.000 |
| fumarate | | | | |
| Total | 249 | 100.0 | 388 | 100.0 |

excipient/API composition

| Material | % w/w | mg/capsule | % w/w | mg/capsule |
|---|---|---|---|---|
| Active ingredient | 0.040 | 0.051 | 0.1036 | 0.202 |
| Mannitol (Pearlitol 100 SD | 92.5 | 115.6 | 92.4 | 180.2 |
| L-HPC LS-B1 | 4.00 | 5.00 | 4.00 | 7.80 |
| HPMC (Methocel E5 Premium) | 2.50 | 3.13 | 2.50 | 4.87 |
| Sodium Stearyl fumarate | 1.000 | 1.251 | 1.001 | 1.950 |
| Total | 100.00 | 125.0 | 100.0 | 195.0 |

| | | |
|---|---|---|
| Number & depth | 1 tamp @ 2 | 1 tamp @ 1 |
| of Tamps | mm depth | mm depth |
| Capsule Details | Green/Grey size 4 | White Size 2 |
| | Quali- G PEG | Quali- G PEG |

TABLE 34
Placebo blend composition during ICH Stability Batches

| | TRI002/89GC Batch Record | | |
|---|---|---|---|
| | g/Batch | | |
| | Sublot 1-for | Sublot 2 | |
| Material | TRI002/89GC-2H | (TRI002/89GC- 4H) | % w/w |
| Diluent | 279 | 486 | 99.0 |
| (TRI002-89DGA) | | | |
| Sodium Stearyl | 2.81 | 4.92 | 1.000 |
| fumarate | | | |
| Total | 282 | 491 | 100.0 |

Excipient/API Composition

| | | mg/capsule | mg/capsule |
|---|---|---|---|
| Material | % w/w | (TRI002/89GC- 4H) | (TRI002/89GC-2H) |
| Mannitol | 92.5 | 115.6 | 206 |
| (Pearlitol 100 SD | | | |
| L-HPC L5-B1 | 4.00 | 5.00 | 8.89 |
| HPMC (Methocel | 2.50 | 3.13 | 5.56 |
| E5 Premium) | | | |
| Sodium Stearyl | 1.000 | 1.25 | 2.22 |
| fumarate | | | |
| Total | 100.0 | 125 | 222 |

| | | |
|---|---|---|
| Number & depth | 1 tamp @ 2 | 1 tamp @ 1 |
| of Tamps | mm depth | mm depth |
| Capsule Details | Green/Grey size 4 | White Size 2 |
| | Quali- G PEG | Quali- G PEG |

### Results

The active batch and a matching placebo granule (Option A) were successfully produced in sublots of 350 g (four Active sublots and eight placebo Sublots). No issues were reported during granulation. The granulation process is summarised in Table 27. The LOD of the different granule is reported in Table 35. The LOD was found to be consistently between 0.3 and 0.7%.

TABLE 35
The LOD of granule sublots for the ICH
Stability Batch (Active and Placebo)

| Batch Code | Sublot | Final LOD (%) |
|---|---|---|
| TRI002/8AGA | 1 | 0.3 |
| | 2 | 0.5 |
| | 3 | 0.7 |
| | 4 | |
| TRI002/8DGA | 1 | 0.6 |
| | 2 | 0.6 |
| | 3 | 0.7 |
| | 4 | 0.5 |
| | 5 | 0.6 |
| | 6 | 0.4 |
| | 7 | 0.6 |
| | 8 | 0.6 |

The active and diluent granules were filled into size 2 capsules so as to achieve a theoretical dose of 200 μg and into size 4 capsules to achieve a theoretical dose of 50 μg dose (Table 33). Matching placebo capsules to the active were also made (Table 34). From the fill weights of individual capsules measured during capsule filling, the capsule showed good uniformity of weight and the following is demonstrated in Table 36:
The average weights of all batches are close to target.
It is noted that the minimum fill weight of the size 4 placebo capsules and the maximum fill weight of the size 2 placebo are outside the target range. However this represents very few capsules—the RSD of the fill weights is low for all batches (~2%), indicating excellent wight uniformity, and so is not a concern.

TABLE 36
Statistics for the individual capsule fill weights for the ICH
Stability Batches (IPC test for each plate of capsules filled)

| | Active | | Placebo | |
|---|---|---|---|---|
| | TRI002/89G50-4H | TRI002/89G200-2H | TRI002/89CG-4 | TRI002/89CG-2G |
| Target fill weight (mg) | 125.0 | 195.0 | 125.8 | 222.0 |
| Average weight (mg) | 125.7 | 196.0 | 122.4 | 221.0 |
| Average weight | 100.6 | 100.5 | 97.3 | 99.5 |
| as % of target | | | | |
| SD | 2.6 | 4.3 | 2.1 | 4.5 |
| % RSD | 2.1 | 2.2 | 1.7 | 2.0 |
| Minimum weight (mg) | 119.3 | 185.1 | [symbol] | 210.8 |
| Maximum weight (mg) | 130.3 | 204.1 | 127.3 | [symbol] |

### T=0 Karl Fischer Results for the ICH Stability Batches

The KF results at T=0 for the ICH Stability Batches are summarised in Table 37. Similar results were obtained for all batches.

TABLE 37
KF summary results
KF

| | Active | | Placebo | |
|---|---|---|---|---|
| Replicate | TRI002/89G50-4H | TRI002/89G200-2H | TRI002/89CG-4H | TRI002/89CG-2H |
| 1 | 0.74 | 0.64 | 0.66 | 0.67 |
| 2 | 0.72 | 0.66 | 0.66 | 0.67 |
| Mean | 0.73 | 0.65 | 0.66 | 0.67 |

### Content Uniformity of the ICH Stability Batches

The system suitability passed the criteria set in the method. The content uniformity results (Table 38) of the active ingredient in both 50 μg and 200 μg capsules meet specification (Acceptance value, AV<15). It was noted that the RSD of active ingredient Content was higher for the 50 μg dose than:
The 200 μg dose, as expected
The 50 μg dose in Part 2 (Table 31).

TABLE 38
Content Uniformity results for the ICH Stability Capsule Batches

| | | | Assay % | | Std | | Acceptance |
|---|---|---|---|---|---|---|---|
| | Repli- | Weight | recovery | Mean | Dev | % | value |
| Batch | cate | (mg) | (T) | (X) | (s) | RSO | (AV) |
| TRI002/ | 1 | 160.35 | 97.1 | 98.7 | 4.2 | 4.3 | 10.1 |
| 89G50- | 2 | 165.31 | 95.0 | | | | |
| 4H | 3 | 152.30 | 99.2 | | | | |
| (50 μg) | 4 | 155.09 | 95.7 | | | | |
| | 5 | 169.59 | 104.7 | | | | |
| | 6 | 165.72 | 107.0 | | | | |
| | 7 | 162.30 | 95.2 | | | | |
| | 8 | 160.87 | 100.7 | | | | |
| | 9 | 160.48 | 95.8 | | | | |
| | 10 | 167.41 | 96.3 | | | | |
| RI002/ | 1 | 265.16 | 100.0 | 98.0 | 3.3 | 3.4 | 2.4 |
| 89G200- | 2 | 254.73 | 97.5 | | | | |
| 2H | 3 | 243.35 | 92.0 | | | | |
| (200 μg) | 4 | 260.41 | 101.6 | | | | |
| | 5 | 263.03 | 101.6 | | | | |
| | 6 | 253.80 | 95.9 | | | | |
| | 7 | 260.01 | 101.8 | | | | |
| | 8 | 245.45 | 94.0 | | | | |
| | 9 | 249.45 | 98.0 | | | | |
| | 10 | 255.41 | 97.9 | | | | |

### T=0 Appearance of the OCH Stability Batches

All capsules appear to be as expected: intact and free from defects, as summarised in Table 39.

TABLE 39
Summary of capsule initial appearance

| Formulation | | Appearance |
|---|---|---|
| Active | TRI002/89G50-4H | Opaque green/grey size 4 capsule |
| | (50 μg) | with no physical defects |
| | TRI002/89G200-2H | Opaque size 2 white capsule with |
| | (200 μg) | no physical defects |
| Placebo | TRI002/89GC-4H | Opaque green/grey size 4 capsule |
| | (Placebo for 50 μg) | with no physical defects. |
| | TRI002/89GC-2H | Opaque size 2 white capsule |
| | (Placebo for 200 μg) | with no physical defects |

### T=0 Assay and Related Substances of the ICH Stability Batches

### Assay

The T=0 assay results are shown in Table 40. The mean assay was found to be 97.1% and 98.3% for 50 μg and 200 μg respectively, i.e. slightly below target, similar to that found in the Content Uniformity results (Table 38).

### Related Substances (RS)

The final results for related substances are shown in Table 41. Overall, no individual related substance exceeded 0.23% for both 50 μg and 200 μg, i.e. well within the target specification (<1%)

TABLE 40
Final Results for T = 0 Assay for the ICH Stability Batches

| Dose | Replicate | Assay (% label claim) |
|---|---|---|
| 50 μg | 1 | 94.4 |
| (TRI002/89G50-4H) | 2 | 99.7 |
| | Mean | 97.1 |
| | Replicate | 5.3 |
| | Agreement | |
| 200 μg | 1 | 97.7 |
| (TRI002/89G200-2H) | 2 | 98.9 |
| | Mean | 98.3 |
| | Replicate | 1.2 |
| | Agreement | |

TABLE 41
T = 0 Related substances for the ICH Stability Batches

| Dose | Replicate | RRT 0.80 | RRT 0.94 | RRT 0.92 | RRT 1.04 | RRT 1.17 | RRT 1.40 | Total |
|---|---|---|---|---|---|---|---|---|
| (TRI002/ | 1 | <0.10 | 0.22 | <0.10 | 0.10 | <0.10 | <0.10 | 0.32 |
| 89G50-4H | | | | | | | | |
| (50 μg)* | | | | | | | | |
| TRI002/ | 1 | <0.10 | 0.22 | 0.11 | 0.16 | <0.10 | <0.10 | 0.50 |
| 89G200-2H | 2 | <0.10 | 0.23 | 0.11 | 0.18 | <0.10 | <0.10 | 0.53 |
| (200 μg) | | | | | | | | |

*This was only carried in one replicate due to the limited number of capsules available

### T=0 Dissolution

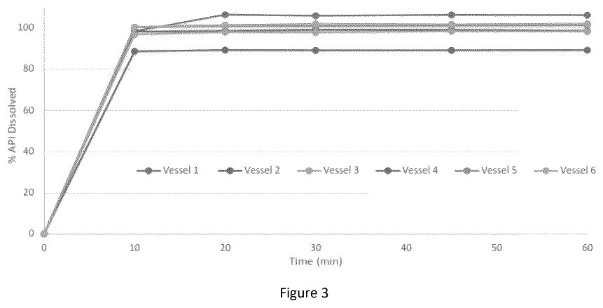

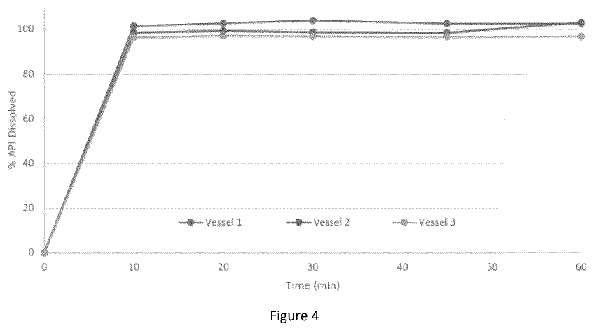

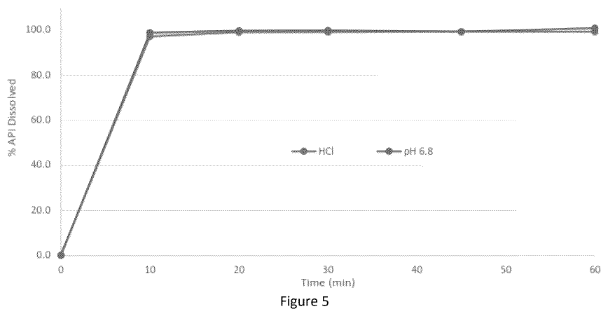

### TRI002/89G50-4H: 50 μg Dose (FIG. 3 to FIG. 5)

In both 0.1M HCl and pH 6.8 phosphate buffer, essentially all of the active ingredient was released within 10 minutes of dissolution, i.e. meets specification (>80% in 45 min in HCl). In pH 6.8 phosphate buffer, acceptable active ingredient release was achieved. Unlike Part 2 2 (FIG. 1 and FIG. 2), there was no delay in the active ingredient release. The improved dissolution here is due to replacing the Quali-V (HPMC) capsules with Quali-G (gelatin). As expected, the dissolution of hard gelatin capsules is not reduced by the introduction of potassium ions in the media.

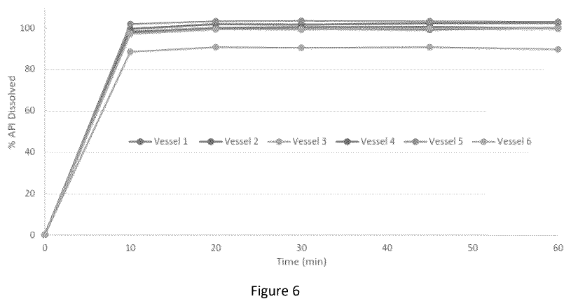

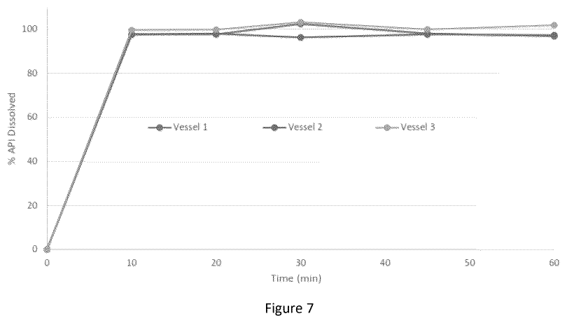

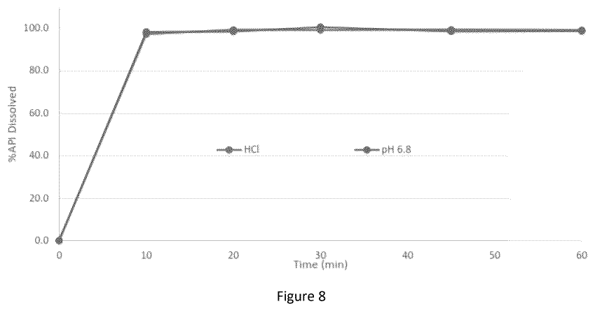

### TRI002/89G200-2H: 200 μg (FIG. 6 to FIG. 8)

In both HCl and pH 6.8 phosphate buffer the active ingredient was essentially fully released within the first 10 minutes. The drug release was consistent in all vessels and almost identical release profiles was seen in both HCl and pH 6.8 phosphate buffer.

### Microbiological Analysis

The two active batches (low and high dose) and matching placebo meet the specification for the microbiological results as summarised in Table 42.

TABLE 42
Microbiological Analysis results summary

| | | Active | | Placebo | |
|---|---|---|---|---|---|
| | | (TRI002/ | (TRI002/ | (TRI002/ | (TRI002/ |
| | | 89G50-4H) | 89G200-2H) | 89GC-4H) | 89GC-2H) |
| Analysis | Specification | 50 μg | 200 μg | 50 μg | 200 μg |
| Total Aerobic | Not more than | <10 cfu/g | <10 cfu/g | 72 cfu/g | 45 cfu/g |
| Microbial Count | 2000 cfu/g | | | | |
| Total Combined Yeast | Not more than | <10 cfu/g | | | |
| and Mould Count | 200 cfu/g | | | | |

| | | |
|---|---|---|
| Escherichia Coli | Absent in 1 g | Absent in 1 g |

### Conclusions from Parts 1-3

Both HPMC and PVP were shown to be suitable as binders. However, granules made with PVP at the High Granulation Extent failed the Blend Uniformity specifications (<15 AV). HPMC binder, on the other hand, passed Blend Uniformity (<15 AV) at both High and Low Extents of Granulation. Based on the superior mean and RSD of active ingredient content for Option A (High Granulation Extent batch in Part 2), this Extent was selected for the ICH Stability Batches over Option B. The Extent of Granulation had no impact on the dissolution rate.

### Formulation Process

Both granulation and encapsulation were successfully preformed. The High Granulation Extent batch appear to be denser than the Low Granulation Extent batch (0.43 g/mL and 0.39 g/mL for High and Low Granulation Extent, respectively, as expected). Furthermore, the High Granulation Extent batch had a lower RSD of active ingredient content compared to the Low Granulation Extent batch, indicating better Content Uniformity. However, the dissolution was identical at both Granulation extents i.e. in target (>80% active ingredient dissolved in 45 minutes). Based on these findings, the High Granulation Extent was selected to produce the ICH capsules.

### Assay and Related Substances/Content Uniformity

The area of the individual related substances (NMT 0.23%) was well within specification (<1.0%) at T=0 and so process-related API degradation is not a concern. Mean assay results for both active batches are within ±2.5% of nominal. Content Uniformity is acceptable (AV<15, i.e. 10.1 for 50 μg and 8.4 for 200 μg)

### Dissolution/Microbiological Analysis

The QC dissolution results in the 0.1M HCl medium and pH 6.8 phosphate buffer dissolution medium dissolution showed essentially complete release at 10-20 minutes. All four capsules batches met specifications for microbiological analysis.

1. A plurality of granules comprising:
a. N-(2-(tert-butyl)-1-((4,4-difluorocyclohexyl)methyl)-1H-benzimidazol-5-yl)ethanesulfonamide, or a pharmaceutically acceptable salt thereof; and
b. at least one pharmaceutically acceptable excipient.

2. The plurality of granules according to claim 1, wherein the pharmaceutically acceptable excipient is selected from a diluent, disintegrant and binder.

3. The plurality of granules according to any preceding claim, wherein the plurality of granules comprise a diluent and binder.

4. The plurality of granules according to any preceding claim, wherein the plurality of granules comprise a diluent, disintegrant and binder.

5. The plurality of granules according to any preceding claim, wherein N-(2-(tert-butyl)-1-((4,4-difluorocyclohexyl)methyl)-1H-benzimidazol-5-yl)ethanesulfonamide, or a pharmaceutically acceptable salt thereof, is present in the plurality of granules in an amount of less than 0.5% w/w by weight of the total weight of the plurality of granules, such as less than 0.4, 0.3, 0.2, 0.15, 0.14, 0.13, 0.12, 0.11, 0.10, 0.08, 0.06, or 0.05% w/w.

6. The plurality of granules according to claims 2 to 5, wherein the diluent is present in the plurality of granules in an amount of at least 70% w/w by weight of the total weight of the plurality of granules, such as at least 75, 80, 85, or 90% w/w.

7. The plurality of granules according to claims 2 to 6 wherein the disintegrant is present in the plurality of granules in an amount of at least 0.5% w/w by weight of the total weight of the plurality of granules, such as at least 1, 2, or 3% w/w.

8. The plurality of granules according to claims 2 to 7, wherein the binder is present in the plurality of granules in an amount of at least 0.5% w/w by weight of the total weight of the plurality of granules, such as at least 1 or 2% w/w.

9. The plurality of granules according to claims 2 to 8, wherein the diluent is mannitol.

10. The plurality of granules according to claims 2 to 9, wherein the disintegrant is selected from low-substituted hydroxypropyl cellulose, sodium starch glycolate and croscarmellose sodium.

11. The plurality of granules according to claim 10, wherein the disintegrant is low-substituted hydroxypropyl cellulose.

12. The plurality of granules according to claims 2 to 11, wherein the binder is selected from hydroxypropyl methyl cellulose and povidone.

13. The plurality of granules according to claim 12, wherein the binder is hydroxypropyl methyl cellulose.

14. The plurality of granules according to claim 13, wherein the hydroxypropyl methyl cellulose is 6 cps grade hydroxypropyl methyl cellulose.

15. The plurality of granules according to any preceding claim, wherein the mean size of the granules is less than 1200 μm.

16. The plurality of granules according to any preceding claim, wherein the bulk density of the granules is between about 0.1 and about 0.7 g/mL, such as between about 0.2 and about 0.6 g/mL, or between about 0.3 and about 0.5 g/mL.

17. The plurality of granules according to any preceding claim, further comprising a lubricant.

18. The plurality of granules according to claim 17, wherein the lubricant is sodium stearyl fumarate.

19. A pharmaceutical composition for oral use comprising a plurality of granules according to claims 1 to 18.

20. The pharmaceutical composition according to claim 19, wherein the pharmaceutical composition is a capsule or tablet.

21. The pharmaceutical composition according to claim 19, wherein the pharmaceutical composition is a capsule.

22. The pharmaceutical composition according to claim 21, wherein the pharmaceutical composition is a capsule comprising a hydroxypropyl methyl cellulose or gelatin capsule shell.

23. The pharmaceutical composition according to claims 19 to 22, wherein the pharmaceutical composition is an immediate release pharmaceutical composition.

24. The pharmaceutical composition according to claim 23 wherein at least 75% of the total amount of the N-(2-(tert-butyl)-1-((4,4-difluorocyclohexyl)methyl)-1H-benzimidazol-5-yl)ethanesulfonamide contained in the pharmaceutical composition is released within the first 45 minutes when the pharmaceutical composition is subjected to an in vitro dissolution test employing 0.1 M HCl or pH 6.8 phosphate buffer as the dissolution medium and the dissolution profile is determined as described in the United States Pharmacopoeia at 37° C. using a rotation speed of 50 rpm.

25. The pharmaceutical composition according to claims 19 to 24, wherein when the pharmaceutical composition is subjected to a content uniformity test as described in the European Pharmacopeia the acceptance value is less than 15.

26. The pharmaceutical composition according to claims 19 to 25 wherein N-(2-(tert-butyl)-1-((4,4-difluorocyclohexyl)methyl)-1H-benzimidazol-5-yl)ethanesulfonamide, or a pharmaceutically acceptable salt thereof, is stable in the pharmaceutical composition for at least 2 weeks, 1, 2, 3, 6, 12, or 24 months.

27. A process to prepare a plurality of granules, wherein the process comprises:
a. mixing a binder and diluent together;
b. adding a primary granulation fluid to the mixture from step a. and mixing until granules are formed; and
c. drying the mixture from step b;
wherein N-(2-(tert-butyl)-1-((4,4-difluorocyclohexyl)methyl)-1H-benzimidazol-5-yl)ethanesulfonamide, or a pharmaceutically acceptable salt thereof, is either mixed with the binder and diluent in step a. or dissolved in the primary granulation fluid.

28. The process according to claim 27, wherein N-(2-(tert-butyl)-1-((4,4-difluorocyclohexyl)methyl)-1H-benzimidazol-5-yl)ethanesulfonamide, or a pharmaceutically acceptable salt thereof, is mixed with the binder and diluent in step a.

29. The process according to claim 27 or 28, wherein step a. further comprises mixing a disintegrant together with the binder and diluent.

30. The process according to claims 27 to 29, wherein N-(2-(tert-butyl)-1-((4,4-difluorocyclohexyl)methyl)-1H-benzimidazol-5-yl)ethanesulfonamide, or a pharmaceutically acceptable salt thereof, has a solubility of at least 0.6 mg/mL in the primary granulation fluid at room temperature.

31. The process according to claims 27 to 30, wherein the primary granulation fluid is selected from ethanol, 0.1 M HCl, pH 3 citric acid buffer and 5% aqueous SLS.

32. The process according to claim 31, wherein the primary granulation fluid is 0.1 M HCl.

33. The process according to claims 27 to 32, wherein in step b. the primary granulation fluid is added at a rate of about 2 to about 10 mL/min, preferably about 4 to about 7 mL/min, more preferably about 5 to about 6 mL/min.

34. The process according to claims 27 to 33, wherein in step b. the mixture is mixed at high speed.

35. The process according to claims 27 to 34, wherein in step c. the granules are dried at an elevated temperature.

36. The process according to claims 27 to 35, wherein the elevated temperature is greater than 30° C., such as greater than 40° C., greater than 50° C.

37. The process according to claims 27 to 36, wherein the process further comprises adding a secondary granulation fluid to step b.

38. The process according to claim 37, wherein the secondary granulation fluid selected from ethanol, 0.1 M HCl, pH 3 citric acid buffer and 5% aqueous SLS.

39. The process according to claim 38, wherein the secondary granulation fluid is 0.1 M HCl.

40. The process according to claims 37 to 39, wherein the secondary fluid is added over a period of at least 1 minute, such as at least 2 minutes, at least 5 minutes or at least 8 minutes.

41. The pharmaceutical composition according to claims 19 to 26 for use as a medicament.

42. The pharmaceutical composition according to claims 19 to 26 for use in a disease state or disorder in which dysfunction of CB₁ and/or CB₂ receptors are present or implicated.

43. The pharmaceutical composition according to claims 19 to 26 for use in the treatment of an anorexia associated condition; a cachexia associated condition; or anorexia nervosa.
